SEQUENCE LISTING (301 sequences)

SEQ ID NO: 1 - protein, 105 aa, Homo Sapiens
MRSFFSFLGEAFDGARDMWRAYSDMREANYIGSDKYFHARGNYDAAKRGPGGVWAAEAISNARENIQRFFGRGAEDSLADQAANEWGRSGKDPNHFRPAGLPEKY

SEQ ID NO: 2 - protein, 4 aa, Homo Sapiens
MRSF

SEQ ID NO: 3 - protein, 5 aa, Homo Sapiens
MRSFF

SEQ ID NO: 4 - protein, 6 aa, Homo Sapiens
MRSFFS

SEQ ID NO: 5 - protein, 7 aa, Homo Sapiens
MRSFFSF

SEQ ID NO: 6 - protein, 8 aa, Homo Sapiens
MRSFFSFL

SEQ ID NO: 7 - protein, 9 aa, Homo Sapiens
MRSFFSFLG

SEQ ID NO: 8 - protein, 10 aa, Homo Sapiens
MRSFFSFLGE

SEQ ID NO: 9 - protein, 11 aa, Homo Sapiens
MRSFFSFLGEA

SEQ ID NO: 10 - protein, 12 aa, Homo Sapiens
MRSFFSFLGEAF

SEQ ID NO: 11 - protein, 13 aa, Homo Sapiens
MRSFFSFLGEAFD

SEQ ID NO: 12 - protein, 14 aa, Homo Sapiens
MRSFFSFLGEAFDG

SEQ ID NO: 13 - protein, 15 aa, Homo Sapiens
MRSFFSFLGEAFDGA

SEQ ID NO: 14 - protein, 16 aa, Homo Sapiens
MRSFFSFLGEAFDGAR

SEQ ID NO: 15 - protein, 17 aa, Homo Sapiens
MRSFFSFLGEAFDGARD

SEQ ID NO: 16 - protein, 18 aa, Homo Sapiens
MRSFFSFLGEAFDGARDM

SEQ ID NO: 17 - protein, 19 aa, Homo Sapiens
MRSFFSFLGEAFDGARDMW

SEQ ID NO: 18 - protein, 20 aa, Homo Sapiens
MRSFFSFLGEAFDGARDMWR

SEQ ID NO: 19 - protein, 21 aa, Homo Sapiens
MRSFFSFLGEAFDGARDMWRA

SEQ ID NO: 20 - protein, 22 aa, Homo Sapiens
MRSFFSFLGEAFDGARDMWRAY

SEQ ID NO: 21 - protein, 23 aa, Homo Sapiens
MRSFFSFLGEAFDGARDMWRAYS

SEQ ID NO: 22 - protein, 24 aa, Homo Sapiens
MRSFFSFLGEAFDGARDMWRAYSD

SEQ ID NO: 23 - protein, 25 aa, Homo Sapiens
MRSFFSFLGEAFDGARDMWRAYSDM

SEQ ID NO: 24 - protein, 26 aa, Homo Sapiens
MRSFFSFLGEAFDGARDMWRAYSDMR

SEQ ID NO: 25 - protein, 27 aa, Homo Sapiens
MRSFFSFLGEAFDGARDMWRAYSDMRE

SEQ ID NO: 26 - protein, 28 aa, Homo Sapiens
MRSFFSFLGEAFDGARDMWRAYSDMREA

SEQ ID NO: 27 - protein, 29 aa, Homo Sapiens
MRSFFSFLGEAFDGARDMWRAYSDMREAN

SEQ ID NO: 28 - protein, 30 aa, Homo Sapiens
MRSFFSFLGEAFDGARDMWRAYSDMREANY

SEQ ID NO: 29 - protein, 31 aa, Homo Sapiens
MRSFFSFLGEAFDGARDMWRAYSDMREANYI

SEQ ID NO: 30 - protein, 32 aa, Homo Sapiens
MRSFFSFLGEAFDGARDMWRAYSDMREANYIG

SEQ ID NO: 31 - protein, 33 aa, Homo Sapiens
MRSFFSFLGEAFDGARDMWRAYSDMREANYIGS

SEQ ID NO: 32 - protein, 34 aa, Homo Sapiens
MRSFFSFLGEAFDGARDMWRAYSDMREANYIGSD

SEQ ID NO: 33 - protein, 35 aa, Homo Sapiens
MRSFFSFLGEAFDGARDMWRAYSDMREANYIGSDK

SEQ ID NO: 34 - protein, 36 aa, Homo Sapiens
MRSFFSFLGEAFDGARDMWRAYSDMREANYIGSDKY

SEQ ID NO: 35 - protein, 37 aa, Homo Sapiens
MRSFFSFLGEAFDGARDMWRAYSDMREANYIGSDKYF

SEQ ID NO: 36 - protein, 38 aa, Homo Sapiens
MRSFFSFLGEAFDGARDMWRAYSDMREANYIGSDKYFH

SEQ ID NO: 37 - protein, 39 aa, Homo Sapiens
MRSFFSFLGEAFDGARDMWRAYSDMREANYIGSDKYFHA

SEQ ID NO: 38 - protein, 40 aa, Homo Sapiens
MRSFFSFLGEAFDGARDMWRAYSDMREANYIGSDKYFHAR

SEQ ID NO: 39 - protein, 41 aa, Homo Sapiens
MRSFFSFLGEAFDGARDMWRAYSDMREANYIGSDKYFHARG

SEQ ID NO: 40 - protein, 42 aa, Homo Sapiens
MRSFFSFLGEAFDGARDMWRAYSDMREANYIGSDKYFHARGN

SEQ ID NO: 41 - protein, 43 aa, Homo Sapiens
MRSFFSFLGEAFDGARDMWRAYSDMREANYIGSDKYFHARGNY

SEQ ID NO: 42 - protein, 44 aa, Homo Sapiens
MRSFFSFLGEAFDGARDMWRAYSDMREANYIGSDKYFHARGNYD

SEQ ID NO: 43 - protein, 45 aa, Homo Sapiens
MRSFFSFLGEAFDGARDMWRAYSDMREANYIGSDKYFHARGNYDA

SEQ ID NO: 44 - protein, 46 aa, Homo Sapiens
MRSFFSFLGEAFDGARDMWRAYSDMREANYIGSDKYFHARGNYDAA

SEQ ID NO: 45 - protein, 47 aa, Homo Sapiens
MRSFFSFLGEAFDGARDMWRAYSDMREANYIGSDKYFHARGNYDAAK

SEQ ID NO: 46 - protein, 48 aa, Homo Sapiens
MRSFFSFLGEAFDGARDMWRAYSDMREANYIGSDKYFHARGNYDAAKR

SEQ ID NO: 47 - protein, 49 aa, Homo Sapiens
MRSFFSFLGEAFDGARDMWRAYSDMREANYIGSDKYFHARGNYDAAKRG

SEQ ID NO: 48 - protein, 50 aa, Homo Sapiens
MRSFFSFLGEAFDGARDMWRAYSDMREANYIGSDKYFHARGNYDAAKRGP

SEQ ID NO: 49 - protein, 51 aa, Homo Sapiens
MRSFFSFLGEAFDGARDMWRAYSDMREANYIGSDKYFHARGNYDAAKRGPG

SEQ ID NO: 50 - protein, 52 aa, Homo Sapiens
MRSFFSFLGEAFDGARDMWRAYSDMREANYIGSDKYFHARGNYDAAKRGPGG

SEQ ID NO: 51 - protein, 53 aa, Homo Sapiens
MRSFFSFLGEAFDGARDMWRAYSDMREANYIGSDKYFHARGNYDAAKRGPGGV

SEQ ID NO: 52 - protein, 54 aa, Homo Sapiens
MRSFFSFLGEAFDGARDMWRAYSDMREANYIGSDKYFHARGNYDAAKRGPGGVW

SEQ ID NO: 53 - protein, 55 aa, Homo Sapiens
MRSFFSFLGEAFDGARDMWRAYSDMREANYIGSDKYFHARGNYDAAKRGPGGVWA

SEQ ID NO: 54 - protein, 56 aa, Homo Sapiens
MRSFFSFLGEAFDGARDMWRAYSDMREANYIGSDKYFHARGNYDAAKRGPGGVWAA

SEQ ID NO: 55 - protein, 57 aa, Homo Sapiens
MRSFFSFLGEAFDGARDMWRAYSDMREANYIGSDKYFHARGNYDAAKRGPGGVWAAE

SEQ ID NO: 56 - protein, 58 aa, Homo Sapiens
MRSFFSFLGEAFDGARDMWRAYSDMREANYIGSDKYFHARGNYDAAKRGPGGVWAAEA

SEQ ID NO: 57 - protein, 59 aa, Homo Sapiens
MRSFFSFLGEAFDGARDMWRAYSDMREANYIGSDKYFHARGNYDAAKRGPGGVWAAEAI

SEQ ID NO: 58 - protein, 60 aa, Homo Sapiens
MRSFFSFLGEAFDGARDMWRAYSDMREANYIGSDKYFHARGNYDAAKRGPGGVWAAEAIS

SEQ ID NO: 59 - protein, 61 aa, Homo Sapiens
MRSFFSFLGEAFDGARDMWRAYSDMREANYIGSDKYFHARGNYDAAKRGPGGVWAAEAISN

SEQ ID NO: 60 - protein, 62 aa, Homo Sapiens
MRSFFSFLGEAFDGARDMWRAYSDMREANYIGSDKYFHARGNYDAAKRGPGGVWAAEAISNA

SEQ ID NO: 61 - protein, 63 aa, Homo Sapiens
MRSFFSFLGEAFDGARDMWRAYSDMREANYIGSDKYFHARGNYDAAKRGPGGVWAAEAISNAR

SEQ ID NO: 62 - protein, 64 aa, Homo Sapiens
MRSFFSFLGEAFDGARDMWRAYSDMREANYIGSDKYFHARGNYDAAKRGPGGVWAAEAISNARE

SEQ ID NO: 63 - protein, 66 aa, Homo Sapiens
MRSFFSFLGEAFDGARDMWRAYSDMREANYIGSDKYFHARGNYDAAKRGPGGVWAAEAISNARENI

SEQ ID NO: 64 - protein, 67 aa, Homo Sapiens
MRSFFSFLGEAFDGARDMWRAYSDMREANYIGSDKYFHARGNYDAAKRGPGGVWAAEAISNARENIQ

SEQ ID NO: 65 - protein, 68 aa, Homo Sapiens
MRSFFSFLGEAFDGARDMWRAYSDMREANYIGSDKYFHARGNYDAAKRGPGGVWAAEAISNARENIQR

SEQ ID NO: 66 - protein, 69 aa, Homo Sapiens
MRSFFSFLGEAFDGARDMWRAYSDMREANYIGSDKYFHARGNYDAAKRGPGGVWAAEAISNARENIQRF

SEQ ID NO: 67 - protein, 70 aa, Homo Sapiens
MRSFFSFLGEAFDGARDMWRAYSDMREANYIGSDKYFHARGNYDAAKRGPGGVWAAEAISNARENIQRFF

SEQ ID NO: 68 - protein, 71 aa, Homo Sapiens
MRSFFSFLGEAFDGARDMWRAYSDMREANYIGSDKYFHARGNYDAAKRGPGGVWAAEAISNARENIQRFFG

SEQ ID NO: 69 - protein, 72 aa, Homo Sapiens
MRSFFSFLGEAFDGARDMWRAYSDMREANYIGSDKYFHARGNYDAAKRGPGGVWAAEAISNARENIQRFFGR

SEQ ID NO: 70 - protein, 73 aa, Homo Sapiens
MRSFFSFLGEAFDGARDMWRAYSDMREANYIGSDKYFHARGNYDAAKRGPGGVWAAEAISNARENIQRFFGRG

SEQ ID NO: 71 - protein, 74 aa, Homo Sapiens
MRSFFSFLGEAFDGARDMWRAYSDMREANYIGSDKYFHARGNYDAAKRGPGGVWAAEAISNARENIQRFFGRGA

SEQ ID NO: 72 - protein, 75 aa, Homo Sapiens
MRSFFSFLGEAFDGARDMWRAYSDMREANYIGSDKYFHARGNYDAAKRGPGGVWAAEAISNARENIQRFFGRGAE

SEQ ID NO: 73 - protein, 76 aa, Homo Sapiens
MRSFFSFLGEAFDGARDMWRAYSDMREANYIGSDKYFHARGNYDAAKRGPGGVWAAEAISNARENIQRFFGRGAED

SEQ ID NO: 74 - protein, 77 aa, Homo Sapiens
MRSFFSFLGEAFDGARDMWRAYSDMREANYIGSDKYFHARGNYDAAKRGPGGVWAAEAISNARENIQRFFGRGAEDS

SEQ ID NO: 75 - protein, 78 aa, Homo Sapiens
MRSFFSFLGEAFDGARDMWRAYSDMREANYIGSDKYFHARGNYDAAKRGPGGVWAAEAISNARENIQRFFGRGAEDSL

SEQ ID NO: 76 - protein, 79 aa, Homo Sapiens
MRSFFSFLGEAFDGARDMWRAYSDMREANYIGSDKYFHARGNYDAAKRGPGGVWAAEAISNARENIQRFFGRGAEDSLA

SEQ ID NO: 77 - protein, 80 aa, Homo Sapiens
MRSFFSFLGEAFDGARDMWRAYSDMREANYIGSDKYFHARGNYDAAKRGPGGVWAAEAISNARENIQRFFGRGAEDSLAD

SEQ ID NO: 78 - protein, 81 aa, Homo Sapiens
MRSFFSFLGEAFDGARDMWRAYSDMREANYIGSDKYFHARGNYDAAKRGPGGVWAAEAISNARENIQRFFGRGAEDSLADQ

SEQ ID NO: 79 - protein, 82 aa, Homo Sapiens
MRSFFSFLGEAFDGARDMWRAYSDMREANYIGSDKYFHARGNYDAAKRGPGGVWAAEAISNARENIQRFFGRGAEDSLADQA

SEQ ID NO: 80 - protein, 83 aa, Homo Sapiens
MRSFFSFLGEAFDGARDMWRAYSDMREANYIGSDKYFHARGNYDAAKRGPGGVWAAEAISNARENIQRFFGRGAEDSLADQAA

SEQ ID NO: 81 - protein, 84 aa, Homo Sapiens
MRSFFSFLGEAFDGARDMWRAYSDMREANYIGSDKYFHARGNYDAAKRGPGGVWAAEAISNARENIQRFFGRGAEDSLADQAAN

SEQ ID NO: 82 - protein, 85 aa, Homo Sapiens
MRSFFSFLGEAFDGARDMWRAYSDMREANYIGSDKYFHARGNYDAAKRGPGGVWAAEAISNARENIQRFFGRGAEDSLADQAANE

SEQ ID NO: 83 - protein, 86 aa, Homo Sapiens
MRSFFSFLGEAFDGARDMWRAYSDMREANYIGSDKYFHARGNYDAAKRGPGGVWAAEAISNARENIQRFFGRGAEDSLADQAANEW

SEQ ID NO: 84 - protein, 87 aa, Homo Sapiens
MRSFFSFLGEAFDGARDMWRAYSDMREANYIGSDKYFHARGNYDAAKRGPGGVWAAEAISNARENIQRFFGRGAEDSLADQAANEWG

SEQ ID NO: 85 - protein, 88 aa, Homo Sapiens
MRSFFSFLGEAFDGARDMWRAYSDMREANYIGSDKYFHARGNYDAAKRGPGGVWAAEAISNARENIQRFFGRGAEDSLADQAANEWGR

SEQ ID NO: 86 - protein, 89 aa, Homo Sapiens
MRSFFSFLGEAFDGARDMWRAYSDMREANYIGSDKYFHARGNYDAAKRGPGGVWAAEAISNARENIQRFFGRGAEDSLADQAANEWGRS

SEQ ID NO: 87 - protein, 90 aa, Homo Sapiens
MRSFFSFLGEAFDGARDMWRAYSDMREANYIGSDKYFHARGNYDAAKRGPGGVWAAEAISNARENIQRFFGRGAEDSLADQAANEWGRSG

SEQ ID NO: 88 - protein, 91 aa, Homo Sapiens
MRSFFSFLGEAFDGARDMWRAYSDMREANYIGSDKYFHARGNYDAAKRGPGGVWAAEAISNARENIQRFFGRGAEDSLADQAANEWGRSGK

SEQ ID NO: 89 - protein, 92 aa, Homo Sapiens
MRSFFSFLGEAFDGARDMWRAYSDMREANYIGSDKYFHARGNYDAAKRGPGGVWAAEAISNARENIQRFFGRGAEDSLADQAANEWGRSGKD

SEQ ID NO: 90 - protein, 93 aa, Homo Sapiens
MRSFFSFLGEAFDGARDMWRAYSDMREANYIGSDKYFHARGNYDAAKRGPGGVWAAEAISNARENIQRFFGRGAEDSLADQAANEWGRSGKDP

SEQ ID NO: 91 - protein, 94 aa, Homo Sapiens
MRSFFSFLGEAFDGARDMWRAYSDMREANYIGSDKYFHARGNYDAAKRGPGGVWAAEAISNARENIQRFFGRGAEDSLADQAANEWGRSGKDPN

SEQ ID NO: 92 - protein, 95 aa, Homo Sapiens
MRSFFSFLGEAFDGARDMWRAYSDMREANYIGSDKYFHARGNYDAAKRGPGGVWAAEAISNARENIQRFFGRGAEDSLADQAANEWGRSGKDPNH

SEQ ID NO: 93 - protein, 96 aa, Homo Sapiens
MRSFFSFLGEAFDGARDMWRAYSDMREANYIGSDKYFHARGNYDAAKRGPGGVWAAEAISNARENIQRFFGRGAEDSLADQAANEWGRSGKDPNHF

SEQ ID NO: 94 - protein, 97 aa, Homo Sapiens
MRSFFSFLGEAFDGARDMWRAYSDMREANYIGSDKYFHARGNYDAAKRGPGGVWAAEAISNARENIQRFFGRGAEDSLADQAANEWGRSGKDPNHFR

SEQ ID NO: 95 - protein, 98 aa, Homo Sapiens
MRSFFSFLGEAFDGARDMWRAYSDMREANYIGSDKYFHARGNYDAAKRGPGGVWAAEAISNARENIQRFFGRGAEDSLADQAANEWGRSGKDPNHFRP

SEQ ID NO: 96 - protein, 99 aa, Homo Sapiens
MRSFFSFLGEAFDGARDMWRAYSDMREANYIGSDKYFHARGNYDAAKRGPGGVWAAEAISNARENIQRFFGRGAEDSLADQAANEWGRSGKDPNHFRPA

SEQ ID NO: 97 - protein, 100 aa, Homo Sapiens
MRSFFSFLGEAFDGARDMWRAYSDMREANYIGSDKYFHARGNYDAAKRGPGGVWAAEAISNARENIQRFFGRGAEDSLADQAANEWGRSGKDPNHFRPAG

SEQ ID NO: 98 - protein, 101 aa, Homo Sapiens
MRSFFSFLGEAFDGARDMWRAYSDMREANYIGSDKYFHARGNYDAAKRGPGGVWAAEAISNARENIQRFFGRGAEDSLADQAANEWGRSGKDPNHFRPAGL

SEQ ID NO: 99 - protein, 102 aa, Homo Sapiens
MRSFFSFLGEAFDGARDMWRAYSDMREANYIGSDKYFHARGNYDAAKRGPGGVWAAEAISNARENIQRFFGRGAEDSLADQAANEWGRSGKDPNHFRPAGLP

SEQ ID NO: 100 - protein, 103 aa, Homo Sapiens
MRSFFSFLGEAFDGARDMWRAYSDMREANYIGSDKYFHARGNYDAAKRGPGGVWAAEAISNARENIQRFFGRGAEDSLADQAANEWGRSGKDPNHFRPAGLPE

SEQ ID NO: 101 - protein, 104 aa, Homo Sapiens
MRSFFSFLGEAFDGARDMWRAYSDMREANYIGSDKYFHARGNYDAAKRGPGGVWAAEAISNARENIQRFFGRGAEDSLADQAANEWGRSGKDPNHFRPAGLPEK

SEQ ID NO: 102 - protein, 104 aa, Homo Sapiens
RSFFSFLGEAFDGARDMWRAYSDMREANYIGSDKYFHARGNYDAAKRGPGGVWAAEAISNARENIQRFFGRGAEDSLADQAANEWGRSGKDPNHFRPAGLPEKY

SEQ ID NO: 103 - protein, 103 aa, Homo Sapiens
SFFSFLGEAFDGARDMWRAYSDMREANYIGSDKYFHARGNYDAAKRGPGGVWAAEAISNARENIQRFFGRGAEDSLADQAANEWGRSGKDPNHFRPAGLPEKY

SEQ ID NO: 104 - protein, 102 aa, Homo Sapiens
FFSFLGEAFDGARDMWRAYSDMREANYIGSDKYFHARGNYDAAKRGPGGVWAAEAISNARENIQRFFGRGAEDSLADQAANEWGRSGKDPNHFRPAGLPEKY

SEQ ID NO: 105 - protein, 101 aa, Homo Sapiens
FSFLGEAFDGARDMWRAYSDMREANYIGSDKYFHARGNYDAAKRGPGGVWAAEAISNARENIQRFFGRGAEDSLADQAANEWGRSGKDPNHFRPAGLPEKY

SEQ ID NO: 106 - protein, 100 aa, Homo Sapiens
SFLGEAFDGARDMWRAYSDMREANYIGSDKYFHARGNYDAAKRGPGGVWAAEAISNARENIQRFFGRGAEDSLADQAANEWGRSGKDPNHFRPAGLPEKY

SEQ ID NO: 107 - protein, 99 aa, Homo Sapiens
FLGEAFDGARDMWRAYSDMREANYIGSDKYFHARGNYDAAKRGPGGVWAAEAISNARENIQRFFGRGAEDSLADQAANEWGRSGKDPNHFRPAGLPEKY

SEQ ID NO: 108 - protein, 98 aa, Homo Sapiens
LGEAFDGARDMWRAYSDMREANYIGSDKYFHARGNYDAAKRGPGGVWAAEAISNARENIQRFFGRGAEDSLADQAANEWGRSGKDPNHFRPAGLPEKY

SEQ ID NO: 109 - protein, 97 aa, Homo Sapiens
GEAFDGARDMWRAYSDMREANYIGSDKYFHARGNYDAAKRGPGGVWAAEAISNARENIQRFFGRGAEDSLADQAANEWGRSGKDPNHFRPAGLPEKY

SEQ ID NO: 110 - protein, 96 aa, Homo Sapiens
EAFDGARDMWRAYSDMREANYIGSDKYFHARGNYDAAKRGPGGVWAAEAISNARENIQRFFGRGAEDSLADQAANEWGRSGKDPNHFRPAGLPEKY

SEQ ID NO: 111 - protein, 95 aa, Homo Sapiens
AFDGARDMWRAYSDMREANYIGSDKYFHARGNYDAAKRGPGGVWAAEAISNARENIQRFFGRGAEDSLADQAANEWGRSGKDPNHFRPAGLPEKY

SEQ ID NO: 112 - protein, 94 aa, Homo Sapiens
FDGARDMWRAYSDMREANYIGSDKYFHARGNYDAAKRGPGGVWAAEAISNARENIQRFFGRGAEDSLADQAANEWGRSGKDPNHFRPAGLPEKY

SEQ ID NO: 113 - protein, 93 aa, Homo Sapiens
DGARDMWRAYSDMREANYIGSDKYFHARGNYDAAKRGPGGVWAAEAISNARENIQRFFGRGAEDSLADQAANEWGRSGKDPNHFRPAGLPEKY

SEQ ID NO: 114 - protein, 92 aa, Homo Sapiens
GARDMWRAYSDMREANYIGSDKYFHARGNYDAAKRGPGGVWAAEAISNARENIQRFFGRGAEDSLADQAANEWGRSGKDPNHFRPAGLPEKY

SEQ ID NO: 115 - protein, 91 aa, Homo Sapiens
ARDMWRAYSDMREANYIGSDKYFHARGNYDAAKRGPGGVWAAEAISNARENIQRFFGRGAEDSLADQAANEWGRSGKDPNHFRPAGLPEKY

SEQ ID NO: 116 - protein, 90 aa, Homo Sapiens
RDMWRAYSDMREANYIGSDKYFHARGNYDAAKRGPGGVWAAEAISNARENIQRFFGRGAEDSLADQAANEWGRSGKDPNHFRPAGLPEKY

SEQ ID NO: 117 - protein, 89 aa, Homo Sapiens
DMWRAYSDMREANYIGSDKYFHARGNYDAAKRGPGGVWAAEAISNARENIQRFFGRGAEDSLADQAANEWGRSGKDPNHFRPAGLPEKY

SEQ ID NO: 118 - protein, 88 aa, Homo Sapiens
MWRAYSDMREANYIGSDKYFHARGNYDAAKRGPGGVWAAEAISNARENIQRFFGRGAEDSLADQAANEWGRSGKDPNHFRPAGLPEKY

SEQ ID NO: 119 - protein, 87 aa, Homo Sapiens
WRAYSDMREANYIGSDKYFHARGNYDAAKRGPGGVWAAEAISNARENIQRFFGRGAEDSLADQAANEWGRSGKDPNHFRPAGLPEKY

SEQ ID NO: 120 - protein, 86 aa, Homo Sapiens
RAYSDMREANYIGSDKYFHARGNYDAAKRGPGGVWAAEAISNARENIQRFFGRGAEDSLADQAANEWGRSGKDPNHFRPAGLPEKY

SEQ ID NO: 121 - protein, 84 aa, Homo Sapiens
YSDMREANYIGSDKYFHARGNYDAAKRGPGGVWAAEAISNARENIQRFFGRGAEDSLADQAANEWGRSGKDPNHFRPAGLPEKY

SEQ ID NO: 122 - protein, 83 aa, Homo Sapiens
SDMREANYIGSDKYFHARGNYDAAKRGPGGVWAAEAISNARENIQRFFGRGAEDSLADQAANEWGRSGKDPNHFRPAGLPEKY

SEQ ID NO: 123 - protein, 82 aa, Homo Sapiens
DMREANYIGSDKYFHARGNYDAAKRGPGGVWAAEAISNARENIQRFFGRGAEDSLADQAANEWGRSGKDPNHFRPAGLPEKY

SEQ ID NO: 124 - protein, 81 aa, Homo Sapiens
MREANYIGSDKYFHARGNYDAAKRGPGGVWAAEAISNARENIQRFFGRGAEDSLADQAANEWGRSGKDPNHFRPAGLPEKY

SEQ ID NO: 125 - protein, 80 aa, Homo Sapiens
REANYIGSDKYFHARGNYDAAKRGPGGVWAAEAISNARENIQRFFGRGAEDSLADQAANEWGRSGKDPNHFRPAGLPEKY

SEQ ID NO: 126 - protein, 79 aa, Homo Sapiens
EANYIGSDKYFHARGNYDAAKRGPGGVWAAEAISNARENIQRFFGRGAEDSLADQAANEWGRSGKDPNHFRPAGLPEKY

SEQ ID NO: 127 - protein, 78 aa, Homo Sapiens
ANYIGSDKYFHARGNYDAAKRGPGGVWAAEAISNARENIQRFFGRGAEDSLADQAANEWGRSGKDPNHFRPAGLPEKY

SEQ ID NO: 128 - protein, 77 aa, Homo Sapiens
NYIGSDKYFHARGNYDAAKRGPGGVWAAEAISNARENIQRFFGRGAEDSLADQAANEWGRSGKDPNHFRPAGLPEKY

SEQ ID NO: 129 - protein, 76 aa, Homo Sapiens
YIGSDKYFHARGNYDAAKRGPGGVWAAEAISNARENIQRFFGRGAEDSLADQAANEWGRSGKDPNHFRPAGLPEKY

SEQ ID NO: 130 - protein, 75 aa, Homo Sapiens
IGSDKYFHARGNYDAAKRGPGGVWAAEAISNARENIQRFFGRGAEDSLADQAANEWGRSGKDPNHFRPAGLPEKY

SEQ ID NO: 131 - protein, 74 aa, Homo Sapiens
GSDKYFHARGNYDAAKRGPGGVWAAEAISNARENIQRFFGRGAEDSLADQAANEWGRSGKDPNHFRPAGLPEKY

SEQ ID NO: 132 - protein, 73 aa, Homo Sapiens
SDKYFHARGNYDAAKRGPGGVWAAEAISNARENIQRFFGRGAEDSLADQAANEWGRSGKDPNHFRPAGLPEKY

SEQ ID NO: 133 - protein, 71 aa, Homo Sapiens
DKYFHARGNYDAAKRGPGGVWAAEAISNAREIQRFFGRGAEDSLADQAANEWGRSGKDPNHFRPAGLPEKY

SEQ ID NO: 134 - protein, 71 aa, Homo Sapiens
KYFHARGNYDAAKRGPGGVWAAEAISNARENIQRFFGRGAEDSLADQAANEWGRSGKDPNHFRPAGLPEKY

SEQ ID NO: 135 - protein, 70 aa, Homo Sapiens
YFHARGNYDAAKRGPGGVWAAEAISNARENIQRFFGRGAEDSLADQAANEWGRSGKDPNHFRPAGLPEKY

SEQ ID NO: 136 - protein, 69 aa, Homo Sapiens
FHARGNYDAAKRGPGGVWAAEAISNARENIQRFFGRGAEDSLADQAANEWGRSGKDPNHFRPAGLPEKY

SEQ ID NO: 137 - protein, 68 aa, Homo Sapiens
HARGNYDAAKRGPGGVWAAEAISNARENIQRFFGRGAEDSLADQAANEWGRSGKDPNHFRPAGLPEKY

SEQ ID NO: 138 - protein, 67 aa, Homo Sapiens
ARGNYDAAKRGPGGVWAAEAISNARENIQRFFGRGAEDSLADQAANEWGRSGKDPNHFRPAGLPEKY

SEQ ID NO: 139 - protein, 66 aa, Homo Sapiens
RGNYDAAKRGPGGVWAAEAISNARENIQRFFGRGAEDSLADQAANEWGRSGKDPNHFRPAGLPEKY

SEQ ID NO: 140 - protein, 65 aa, Homo Sapiens
GNYDAAKRGPGGVWAAEAISNARENIQRFFGRGAEDSLADQAANEWGRSGKDPNHFRPAGLPEKY

SEQ ID NO: 141 - protein, 64 aa, Homo Sapiens
NYDAAKRGPGGVWAAEAISNARENIQRFFGRGAEDSLADQAANEWGRSGKDPNHFRPAGLPEKY

SEQ ID NO: 142 - protein, 63 aa, Homo Sapiens
YDAAKRGPGGVWAAEAISNARENIQRFFGRGAEDSLADQAANEWGRSGKDPNHFRPAGLPEKY

SEQ ID NO: 143 - protein, 62 aa, Homo Sapiens
DAAKRGPGGVWAAEAISNARENIQRFFGRGAEDSLADQAANEWGRSGKDPNHFRPAGLPEKY

SEQ ID NO: 144 - protein, 61 aa, Homo Sapiens
AAKRGPGGVWAAEAISNARENIQRFFGRGAEDSLADQAANEWGRSGKDPNHFRPAGLPEKY

SEQ ID NO: 145 - protein, 60 aa, Homo Sapiens
AKRGPGGVWAAEAISNARENIQRFFGRGAEDSLADQAANEWGRSGKDPNHFRPAGLPEKY

SEQ ID NO: 146 - protein, 59 aa, Homo Sapiens
KRGPGGVWAAEAISNARENIQRFFGRGAEDSLADQAANEWGRSGKDPNHFRPAGLPEKY

SEQ ID NO: 147 - protein, 58 aa, Homo Sapiens
RGPGGVWAAEAISNARENIQRFFGRGAEDSLADQAANEWGRSGKDPNHFRPAGLPEKY

SEQ ID NO: 148 - protein, 57 aa, Homo Sapiens
GPGGVWAAEAISNARENIQRFFGRGAEDSLADQAANEWGRSGKDPNHFRPAGLPEKY

SEQ ID NO: 149 - protein, 56 aa, Homo Sapiens
PGGVWAAEAISNARENIQRFFGRGAEDSLADQAANEWGRSGKDPNHFRPAGLPEKY

SEQ ID NO: 150 - protein, 55 aa, Homo Sapiens
GGVWAAEAISNARENIQRFFGRGAEDSLADQAANEWGRSGKDPNHFRPAGLPEKY

SEQ ID NO: 151 - protein, 54 aa, Homo Sapiens
GVWAAEAISNARENIQRFFGRGAEDSLADQAANEWGRSGKDPNHFRPAGLPEKY

SEQ ID NO: 152 - protein, 53 aa, Homo Sapiens
VWAAEAISNARENIQRFFGRGAEDSLADQAANEWGRSGKDPNHFRPAGLPEKY

SEQ ID NO: 153 - protein, 52 aa, Homo Sapiens
WAAEAISNARENIQRFFGRGAEDSLADQAANEWGRSGKDPNHFRPAGLPEKY

SEQ ID NO: 154 - protein, 51 aa, Homo Sapiens
AAEAISNARENIQRFFGRGAEDSLADQAANEWGRSGKDPNHFRPAGLPEKY

SEQ ID NO: 155 - protein, 51 aa, Homo Sapiens; identical to SEQ ID NO: 154

SEQ ID NO: 156 - protein, 49 aa, Homo Sapiens
EAISNARENIQRFFGRGAEDSLADQAANEWGRSGKDPNHFRPAGLPEKY

SEQ ID NO: 157 - protein, 48 aa, Homo Sapiens
AISNARENIQRFFGRGAEDSLADQAANEWGRSGKDPNHFRPAGLPEKY

SEQ ID NO: 158 - protein, 47 aa, Homo Sapiens
ISNARENIQRFFGRGAEDSLADQAANEWGRSGKDPNHFRPAGLPEKY

SEQ ID NO: 159 - protein, 46 aa, Homo Sapiens
SNARENIQRFFGRGAEDSLADQAANEWGRSGKDPNHFRPAGLPEKY

SEQ ID NO: 160 - protein, 45 aa, Homo Sapiens
NARENIQRFFGRGAEDSLADQAANEWGRSGKDPNHFRPAGLPEKY

SEQ ID NO: 161 - protein, 44 aa, Homo Sapiens
ARENIQRFFGRGAEDSLADQAANEWGRSGKDPNHFRPAGLPEKY

SEQ ID NO: 162 - protein, 43 aa, Homo Sapiens
RENIQRFFGRGAEDSLADQAANEWGRSGKDPNHFRPAGLPEKY

SEQ ID NO: 163 - protein, 42 aa, Homo Sapiens
ENIQRFFGRGAEDSLADQAANEWGRSGKDPNHFRPAGLPEKY

SEQ ID NO: 164 - protein, 41 aa, Homo Sapiens
NIQRFFGRGAEDSLADQAANEWGRSGKDPNHFRPAGLPEKY

SEQ ID NO: 165 - protein, 40 aa, Homo Sapiens
IQRFFGRGAEDSLADQAANEWGRSGKDPNHFRPAGLPEKY

SEQ ID NO: 166 - protein, 39 aa, Homo Sapiens
QRFFGRGAEDSLADQAANEWGRSGKDPNHFRPAGLPEKY

SEQ ID NO: 167 - protein, 38 aa, Homo Sapiens
RFFGRGAEDSLADQAANEWGRSGKDPNHFRPAGLPEKY

SEQ ID NO: 168 - protein, 37 aa, Homo Sapiens
FFGRGAEDSLADQAANEWGRSGKDPNHFRPAGLPEKY

SEQ ID NO: 169 - protein, 35 aa, Homo Sapiens
GRGAEDSLADQAANEWGRSGKDPNHFRPAGLPEKY

SEQ ID NO: 170 - protein, 34 aa, Homo Sapiens
RGAEDSLADQAANEWGRSGKDPNHFRPAGLPEKY

SEQ ID NO: 171 - protein, 33 aa, Homo Sapiens
GAEDSLADQAANEWGRSGKDPNHFRPAGLPEKY

SEQ ID NO: 172 - protein, 32 aa, Homo Sapiens
AEDSLADQAANEWGRSGKDPNHFRPAGLPEKY

SEQ ID NO: 173 - protein, 31 aa, Homo Sapiens
EDSLADQAANEWGRSGKDPNHFRPAGLPEKY

SEQ ID NO: 174 - protein, 30 aa, Homo Sapiens
DSLADQAANEWGRSGKDPNHFRPAGLPEKY

SEQ ID NO: 175 - protein, 29 aa, Homo Sapiens
SLADQAANEWGRSGKDPNHFRPAGLPEKY

SEQ ID NO: 176 - protein, 28 aa, Homo Sapiens
LADQAANEWGRSGKDPNHFRPAGLPEKY

SEQ ID NO: 177 - protein, 27 aa, Homo Sapiens
ADQAANEWGRSGKDPNHFRPAGLPEKY

SEQ ID NO: 178 - protein, 26 aa, Homo Sapiens
DQAANEWGRSGKDPNHFRPAGLPEKY

SEQ ID NO: 179 - protein, 25 aa, Homo Sapiens
QAANEWGRSGKDPNHFRPAGLPEKY

SEQ ID NO: 180 - protein, 24 aa, Homo Sapiens
AANEWGRSGKDPNHFRPAGLPEKY

SEQ ID NO: 181 - protein, 23 aa, Homo Sapiens
ANEWGRSGKDPNHFRPAGLPEKY

SEQ ID NO: 182 - protein, 22 aa, Homo Sapiens
NEWGRSGKDPNHFRPAGLPEKY

SEQ ID NO: 183 - protein, 21 aa, Homo Sapiens
EWGRSGKDPNHFRPAGLPEKY

SEQ ID NO: 184 - protein, 20 aa, Homo Sapiens
WGRSGKDPNHFRPAGLPEKY

SEQ ID NO: 185 - protein, 19 aa, Homo Sapiens
GRSGKDPNHFRPAGLPEKY

SEQ ID NO: 186 - protein, 18 aa, Homo Sapiens
RSGKDPNHFRPAGLPEKY

SEQ ID NO: 187 - protein, 17 aa, Homo Sapiens
SGKDPNHFRPAGLPEKY

SEQ ID NO: 188 - protein, 16 aa, Homo Sapiens
GKDPNHFRPAGLPEKY

SEQ ID NO: 189 - protein, 15 aa, Homo Sapiens
KDPNHFRPAGLPEKY

SEQ ID NO: 190 - protein, 14 aa, Homo Sapiens
DPNHFRPAGLPEKY

SEQ ID NO: 191 - protein, 13 aa, Homo Sapiens
PNHFRPAGLPEKY

SEQ ID NO: 192 - protein, 12 aa, Homo Sapiens
NHFRPAGLPEKY

SEQ ID NO: 193 - protein, 11 aa, Homo Sapiens
HFRPAGLPEKY

SEQ ID NO: 194 - protein, 10 aa, Homo Sapiens
FRPAGLPEKY

SEQ ID NO: 195 - protein, 9 aa, Homo Sapiens
RPAGLPEKY

SEQ ID NO: 196 - protein, 8 aa, Homo Sapiens
PAGLPEKY

SEQ ID NO: 197 - protein, 7 aa, Homo Sapiens
AGLPEKY

SEQ ID NO: 198 - protein, 6 aa, Homo Sapiens
GLPEKY

SEQ ID NO: 199 - protein, 5 aa, Homo Sapiens
LPEKY

SEQ ID NO: 200 - protein, 4 aa, Homo Sapiens
PEKY

SEQ ID NO: 201 - protein, 99 aa, Homo Sapiens; identical to SEQ ID NO: 107

SEQ ID NO: 202 - protein, 97 aa, Homo Sapiens; identical to SEQ ID NO: 109

SEQ ID NO: 203 - protein, 99 aa, Homo Sapiens; identical to SEQ ID NO: 107

SEQ ID NO: 204 - protein, 101 aa, Homo Sapiens; identical to SEQ ID NO: 105

SEQ ID NO: 205 - protein, 100 aa, Homo Sapiens; identical to SEQ ID NO: 106

SEQ ID NO: 206 - protein, 98 aa, Homo Sapiens
FLGEAFDGARDMWRAYSDMREANYIGSDKYFARGNYDAAKRGPGGVWAAEAISNARENIQRFFGRGAEDSLADQAANEWGRSGKDPNHFRPAGLPEKY

SEQ ID NO: 207 - protein, 98 aa, Homo Sapiens; identical to SEQ ID NO: 108

SEQ ID NO: 208 - protein, 97 aa, Homo Sapiens; identical to SEQ ID NO: 109

SEQ ID NO: 209 - protein, 96 aa, Homo Sapiens; identical to SEQ ID NO: 110

SEQ ID NO: 210 - protein, 95 aa, Homo Sapiens; identical to SEQ ID NO: 111

SEQ ID NO: 211 - protein, 94 aa, Homo Sapiens; identical to SEQ ID NO: 112

SEQ ID NO: 212 - protein, 93 aa, Homo Sapiens; identical to SEQ ID NO: 113

SEQ ID NO: 213 - protein, 92 aa, Homo Sapiens; identical to SEQ ID NO: 114

SEQ ID NO: 214 - protein, 91 aa, Homo Sapiens; identical to SEQ ID NO: 115

SEQ ID NO: 215 - protein, 90 aa, Homo Sapiens; identical to SEQ ID NO: 116

SEQ ID NO: 216 - protein, 89 aa, Homo Sapiens; identical to SEQ ID NO: 117

SEQ ID NO: 217 - protein, 88 aa, Homo Sapiens; identical to SEQ ID NO: 118

SEQ ID NO: 218 - protein, 87 aa, Homo Sapiens; identical to SEQ ID NO: 119

SEQ ID NO: 219 - protein, 86 aa, Homo Sapiens; identical to SEQ ID NO: 120

SEQ ID NO: 220 - protein, 85 aa, Homo Sapiens
AYSDMREANYIGSDKYFHARGNYDAAKRGPGGVWAAEAISNARENIQRFFGRGAEDSLADQAANEWGRSGKDPNHFRPAGLPEKY

SEQ ID NO: 221 - protein, 84 aa, Homo Sapiens; identical to SEQ ID NO: 121

SEQ ID NO: 222 - protein, 83 aa, Homo Sapiens; identical to SEQ ID NO: 122

SEQ ID NO: 223 - protein, 82 aa, Homo Sapiens; identical to SEQ ID NO: 123

SEQ ID NO: 224 - protein, 81 aa, Homo Sapiens; identical to SEQ ID NO: 124

SEQ ID NO: 225 - protein, 80 aa, Homo Sapiens; identical to SEQ ID NO: 125

SEQ ID NO: 226 - protein, 79 aa, Homo Sapiens; identical to SEQ ID NO: 126

SEQ ID NO: 227 - protein, 78 aa, Homo Sapiens; identical to SEQ ID NO: 127

SEQ ID NO: 228 - protein, 77 aa, Homo Sapiens; identical to SEQ ID NO: 128

SEQ ID NO: 229 - protein, 76 aa, Homo Sapiens; identical to SEQ ID NO: 129

SEQ ID NO: 230 - protein, 75 aa, Homo Sapiens; identical to SEQ ID NO: 130

SEQ ID NO: 231 - protein, 74 aa, Homo Sapiens; identical to SEQ ID NO: 131

SEQ ID NO: 232 - protein, 73 aa, Homo Sapiens; identical to SEQ ID NO: 132

SEQ ID NO: 233 - protein, 72 aa, Homo Sapiens
DKYFHARGNYDAAKRGPGGVWAAEAISNARENIQRFFGRGAEDSLADQAANEWGRSGKDPNHFRPAGLPEKY

SEQ ID NO: 234 - protein, 71 aa, Homo Sapiens; identical to SEQ ID NO: 134

SEQ ID NO: 235 - protein, 70 aa, Homo Sapiens; identical to SEQ ID NO: 135

SEQ ID NO: 236 - protein, 69 aa, Homo Sapiens; identical to SEQ ID NO: 136

SEQ ID NO: 237 - protein, 68 aa, Homo Sapiens; identical to SEQ ID NO: 137

SEQ ID NO: 238 - protein, 67 aa, Homo Sapiens; identical to SEQ ID NO: 138

SEQ ID NO: 239 - protein, 66 aa, Homo Sapiens; identical to SEQ ID NO: 139

SEQ ID NO: 240 - protein, 65 aa, Homo Sapiens; identical to SEQ ID NO: 140

SEQ ID NO: 241 - protein, 64 aa, Homo Sapiens; identical to SEQ ID NO: 141

SEQ ID NO: 242 - protein, 63 aa, Homo Sapiens; identical to SEQ ID NO: 142

SEQ ID NO: 243 - protein, 62 aa, Homo Sapiens; identical to SEQ ID NO: 143

SEQ ID NO: 244 - protein, 61 aa, Homo Sapiens; identical to SEQ ID NO: 144

SEQ ID NO: 245 - protein, 60 aa, Homo Sapiens; identical to SEQ ID NO: 145

SEQ ID NO: 246 - protein, 59 aa, Homo Sapiens; identical to SEQ ID NO: 146

SEQ ID NO: 247 - protein, 58 aa, Homo Sapiens; identical to SEQ ID NO: 147

SEQ ID NO: 248 - protein, 57 aa, Homo Sapiens; identical to SEQ ID NO: 148

SEQ ID NO: 249 - protein, 56 aa, Homo Sapiens; identical to SEQ ID NO: 149

SEQ ID NO: 250 - protein, 55 aa, Homo Sapiens; identical to SEQ ID NO: 150

SEQ ID NO: 251 - protein, 54 aa, Homo Sapiens; identical to SEQ ID NO: 151

SEQ ID NO: 252 - protein, 53 aa, Homo Sapiens; identical to SEQ ID NO: 152

SEQ ID NO: 253 - protein, 52 aa, Homo Sapiens; identical to SEQ ID NO: 153

SEQ ID NO: 254 - protein, 51 aa, Homo Sapiens; identical to SEQ ID NO: 154

SEQ ID NO: 255 - protein, 50 aa, Homo Sapiens
AEAISNARENIQRFFGRGAEDSLADQAANEWGRSGKDPNHFRPAGLPEKY

SEQ ID NO: 256 - protein, 49 aa, Homo Sapiens; identical to SEQ ID NO: 156

SEQ ID NO: 257 - protein, 48 aa, Homo Sapiens; identical to SEQ ID NO: 157

SEQ ID NO: 258 - protein, 47 aa, Homo Sapiens; identical to SEQ ID NO: 158

SEQ ID NO: 259 - protein, 46 aa, Homo Sapiens; identical to SEQ ID NO: 159

SEQ ID NO: 260 - protein, 45 aa, Homo Sapiens; identical to SEQ ID NO: 160

SEQ ID NO: 261 - protein, 44 aa, Homo Sapiens; identical to SEQ ID NO: 161

SEQ ID NO: 262 - protein, 43 aa, Homo Sapiens; identical to SEQ ID NO: 162

SEQ ID NO: 263 - protein, 42 aa, Homo Sapiens; identical to SEQ ID NO: 163

SEQ ID NO: 264 - protein, 41 aa, Homo Sapiens; identical to SEQ ID NO: 164

SEQ ID NO: 265 - protein, 40 aa, Homo Sapiens; identical to SEQ ID NO: 165

SEQ ID NO: 266 - protein, 39 aa, Homo Sapiens; identical to SEQ ID NO: 166

SEQ ID NO: 267 - protein, 38 aa, Homo Sapiens; identical to SEQ ID NO: 167

SEQ ID NO: 268 - protein, 37 aa, Homo Sapiens; identical to SEQ ID NO: 168

SEQ ID NO: 269 - protein, 36 aa, Homo Sapiens
FGRGAEDSLADQAANEWGRSGKDPNHFRPAGLPEKY

SEQ ID NO: 270 - protein, 35 aa, Homo Sapiens; identical to SEQ ID NO: 169

SEQ ID NO: 271 - protein, 34 aa, Homo Sapiens; identical to SEQ ID NO: 170

SEQ ID NO: 272 - protein, 33 aa, Homo Sapiens; identical to SEQ ID NO: 171

SEQ ID NO: 273 - protein, 32 aa, Homo Sapiens; identical to SEQ ID NO: 172

SEQ ID NO: 274 - protein, 31 aa, Homo Sapiens; identical to SEQ ID NO: 173

SEQ ID NO: 275 - protein, 30 aa, Homo Sapiens; identical to SEQ ID NO: 174

SEQ ID NO: 276 - protein, 29 aa, Homo Sapiens
SLADQAANEWGRSGKDPNHFRPAGLPEKY

SEQ ID NO: 277 - protein, 28 aa, Homo Sapiens
LADQAANEWGRSGKDPNHFRPAGLPEKY

SEQ ID NO: 278 - protein, 27 aa, Homo Sapiens
ADQAANEWGRSGKDPNHFRPAGLPEKY

SEQ ID NO: 279 - protein, 26 aa, Homo Sapiens
DQAANEWGRSGKDPNHFRPAGLPEKY

SEQ ID NO: 280 - protein, 25 aa, Homo Sapiens
QAANEWGRSGKDPNHFRPAGLPEKY

SEQ ID NO: 281 - protein, 24 aa, Homo Sapiens
AANEWGRSGKDPNHFRPAGLPEKY

SEQ ID NO: 282 - protein, 23 aa, Homo Sapiens
ANEWGRSGKDPNHFRPAGLPEKY

SEQ ID NO: 283 - protein, 22 aa, Homo Sapiens
NEWGRSGKDPNHFRPAGLPEKY

SEQ ID NO: 284 - protein, 21 aa, Homo Sapiens
EWGRSGKDPNHFRPAGLPEKY

SEQ ID NO: 285 - protein, 20 aa, Homo Sapiens
WGRSGKDPNHFRPAGLPEKY

SEQ ID NO: 286 - protein, 19 aa, Homo Sapiens
GRSGKDPNHFRPAGLPEKY

SEQ ID NO: 287 - protein, 18 aa, Homo Sapiens
RSGKDPNHFRPAGLPEKY

SEQ ID NO: 288 - protein, 17 aa, Homo Sapiens
SGKDPNHFRPAGLPEKY

SEQ ID NO: 289 - protein, 16 aa, Homo Sapiens
GKDPNHFRPAGLPEKY

SEQ ID NO: 290 - protein, 15 aa, Homo Sapiens
KDPNHFRPAGLPEKY

SEQ ID NO: 291 - protein, 14 aa, Homo Sapiens
DPNHFRPAGLPEKY

SEQ ID NO: 292 - protein, 13 aa, Homo Sapiens
PNHFRPAGLPEKY

SEQ ID NO: 293 - protein, 12 aa, Homo Sapiens
NHFRPAGLPEKY

SEQ ID NO: 294 - protein, 11 aa, Homo Sapiens
HFRPAGLPEKY

SEQ ID NO: 295 - protein, 10 aa, Homo Sapiens
FRPAGLPEKY

SEQ ID NO: 296 - protein, 9 aa, Homo Sapiens
RPAGLPEKY

SEQ ID NO: 297 - protein, 8 aa, Homo Sapiens
PAGLPEKY

SEQ ID NO: 298 - protein, 7 aa, Homo Sapiens
AGLPEKY

SEQ ID NO: 299 - protein, 6 aa, Homo Sapiens
GLPEKY

SEQ ID NO: 300 - protein, 5 aa, Homo Sapiens
LPEKY

SEQ ID NO: 301 - protein, 4 aa, Homo Sapiens
PEKY

(19) United States
(12) Patent Application Publication
(10) Pub. No.: US 2005/0085629 A1
(43) Pub. Date: Apr. 21, 2005

(54) Utilization of FPRL1 as a functional receptor by serum amyloid A (SAA)

(76) Inventors: Ji Ming Wang, Frederick, MD (US); Joost J. Oppenheim, Bethesda, MD (US); Shao-Bo Su, Rockville, MD (US); Wang Hua Gong, Frederick, MD (US); Ji-Liang Gao, Frederick, MD (US); Philip M. Murphy, Rockville, MD (US)

Correspondence Address: KNOBBE, MARTENS, OLSON & BEAR, LLP, 2040 MAIN STREET, FOURTEENTH FLOOR, IRVINE, CA, 92614, US

(21) Appl. No.: 10/987,664

(22) Filed: Nov. 12, 2004

Related U.S. Application Data
(63) Continuation of application No. 10/099,782, filed on Mar. 14, 2002.
Application No. 10/099,782 is a continuation of application No. PCT/US99/21770, filed on Sep. 22, 1999.

(51) Int. Cl. G01N 33/53; C07H 21/04; C07K 14/47

(52) U.S. Cl. 530/395; 435/7.1; 435/69.1; 435/320.1; 435/325; 530/324; 536/23.5

(57) ABSTRACT

The present invention relates to the discovery that serum amyloid A (SAA) is a ligand for the FPRL1 receptor. Disclosed herein, are novel biological tools for the study of SAA/FPRL1 complex assembly and prophylactics, therapeutics, and methods of use of the foregoing, which modulate the association of SAA with FPRL1 and thereby effect responses including, but not limited to, signal transduction, chemotaxis, leukocyte migration, immune system response, amyloidosis, inflammatory response, infection, organ rejection, arthritis, atherosclerosis, and neoplasia.

### CROSS-REFERENCE TO RELATED APPLICATIONS

This application is a continuation and claims the benefit of priority of U.S. Pat. Appl. Ser. No. 10/099,782 filed Mar. 14, 2002, which is a continuation and claims the benefit of priority of International Application No. PCT/US99/21770 filed Sep. 22, 1999, designating the United States of America and published in English on Mar. 29, 2001, as WO 01/21188, both of which are hereby expressly incorporated by reference in their entireties.

### FIELD OF INVENTION

The present invention relates to the discovery that serum amyloid A (SAA) is a ligand for the FPRL1 receptor. Disclosed herein are novel biological tools for the study of SAA/FPRL1 complex assembly and prophylactics, therapeutics, and methods of use of the foregoing, which modulate the association of SAA with FPRL1 and thereby effect cellular responses.

### BACKGROUND OF THE INVENTION

Serum amyloid A (SAA), an acute phase protein, is normally present in serum at 0.1: M levels, but increases by 1000 fold in systemic inflammatory conditions. (Sipe, J. D., Immunophysiology: The role of cells and cytokines in immunity and inflammation, 259-273 (1990); Kisilevsky, R., Medical Hypotheses, 35:337-341 (1991); Skinner, M., J. Inten. Med., 232:513-514 (1992); and Malle, E. and F. C. De Beer, Eur. J. Clin. Invest., 26:427-435 (1996)). It has been proposed that SAA is involved in lipid transportation and metabolism. (Sipe, J. D., Immunophysiology: The role of cells and cytokines in immunity and inflammation, 259-273 (1990); Kisilevsky, R., Medical Hypotheses, 35:337-341 (1991); and Skinner, M., J. Inten. Med., 232:513-514 (1992)). Chronic inflammatory conditions with elevated serum SAA can culminate in amyloidosis, characterized by deposition of “amyloid” fibrils in tissues and associated with progressive destruction of organ function. (Kisilevsky, R., Medical Hypotheses, 35:337-341 (1991); Skinner, M., J. Inten. Med., 232:513-514 (1992); and Malle, E. and F. C. De Beer, Eur. J. Clin. Invest., 26:427-435 (1996)). Although a number of acute phase proteins are known to modulate host immune responses, recombinant human (rh) SAA has exhibited chemoattractant activity for human monocytes, neutrophils and T lymphocytes in vitro. (Badolato et al., J. Exp. Med., 180:203-209 (1994); and Xu et al., J. Immunol., 155:1184-1190 (1995)). Further, rhSAA induces infiltration of phagocytic cells and T lymphocytes into injection sites in mice. (Badolato et al., J. Exp. Med., 180:203-209 (1994); and Xu et al., J. Immunol., 155:1184-1190 (1995)). Thus, many in the art recognize that SAA plays a role in cellular responses including, but not limited to, leukocyte migration, immune system response, amyloidosis, inflammatory response, infection, organ rejection, arthritis, atherosclerosis, and neoplasia.

Since SAA induces significant Ca²⁺ mobilization in phagocytes (Badolato et al., J. Immunol., 155:4004-4010 (1995)) and both its chemotactic and Ca²⁺ mobilizing effects are inhibited by pretreatment with pertussis toxin, SAA is thought to mediate cellular responses through G-protein-coupled receptors. (Xu et al., J. Immunol., 155:1184-1190 (1995); and Badolato et al., J. Immunol., 155:4004-4010 (1995)). Despite considerable effort, however, investigators have failed to identify the SAA receptor on human phagocytic cells that mediate the aforementioned cellular responses.

### SUMMARY OF THE INVENTION

The acute phase protein serum amyloid A (SAA) is a potent chemoattractant for human leukocytes in vitro and mouse phagocytes in vivo. To identify the signaling mechanisms, we have evaluated patterns of cross-desensitization between SAA and other leukocyte chemoattractants. During these studies, we discovered that high concentrations of the chemotactic bacterial peptide, N-formyl-methionyl-leucyl-phenylalanine (fMLP), was able to specifically attenuate SAA induced Ca²⁺ mobilization in human phagocytes. With this data in hand, we realized that SAA uses a low affinity fMLP receptor.

Further investigation revealed that SAA selectively induces Ca²⁺ mobilization and migration of cells that were transfected to express a seven-transmembrane G proten coupled receptor, FPRL1. FPRL1 is a human phagocyte receptor with low affinity for fMLP and a high affinity for lipoxin A4, a lipid mediator. More evidence that FPRL1 was the SAA receptor on human phagocytes was obtained from experiments that characterized the ability of radiolabeled SAA to bind to human phagocytes and 293 cells transfected to express FPRL1. In these studies, we found that SAA exhibits a high affinity for the FPRL1 receptor but is not a ligand or agonist for the high affinity fmLP receptor, FPR. Significantly, over the course of several experiments, we have discovered that a SAA/FPRL1 complex can mediate signal transduction, leukocyte migration and activation, which account for the many activities of SAA in immune system response, amyloidosis, inflammatory response, infection, organ rejection, arthritis, atherosclerosis, and neoplasia.

### BRIEF DESCRIPTION OF THE DRAWINGS

FIG. 1A-C Cross-desensitization of Ca²⁺ mobilization in human monocytes between SAA and fMLP. Fura-2 loaded monocytes were sequentially stimulated with SAA and fMLP (panel A) or vice versa (panel B and C), and the ratio of fluorescence at 340 and 380 nm wavelength was recorded and calculated with the FLWinLab program.

FIGS. 2A-F Calcium mobilization in FPRL1 transfected HEK 293 cells. FPRL1/293 cells were loaded with Fura-2 and were stimulated with various concentrations of fMLP (FIG. 2A) or SAA (FIG. 2B). SAA does not induce Ca²⁺ mobilization in FPR expressing 293 cells or mock transfected 293 cells (FIGS. 2C-2D). FIG. 2E-2F show the sequential stimulation of FPRL1/293 cells with SAA and fMLP or vice versa.

FIGS. 3A-C Chemotactic activity of SAA for human monocytes and cells transfected to express chemoattractant receptors. Different concentrations of SAA were placed in the lower wells of the chemotaxis chamber. Monocytes, FPRL1/293 cells or FPR expressing ETFR cells were placed in the upper wells. After incubation, the cells migrated across the polycarbonate filter were counted and photographed; SAA 0.8: M, fMLP 100 nM. The cell migration was expressed as chemotaxis index representing the fold increase of the cells migrating in response to stimulants over control medium. FIG. 3A: migration of FPRL1/293 cells in response to SAA and fMLP. FIG. 3B: migration of FPR expressing ETFR cells in response to SAA and fMLP. FIG. 3C: effect of HDL on SAA induced FPRL1/293 cell migration. HDL at 1000: g/ml mixed with 0.8: M SAA was preincubated at 37° C. for 4 h. The mixture was then tested for chemotactic activity on FPRL1/293 cells. The HDL/SAA mixture without preincubation was also tested for chemotactic activity and yielded similar results.

FIG. 4A-D Binding of ¹²⁵I-SAA to human monocytes and FPRL1/293 cells. rhSAA was radio-iodinated by the chloramine T method and the binding of ¹²⁵I-SAA to FPRL1/293 cells (panel A) or monocytes (panel B) was measured by adding a constant concentration of ¹²⁵I-SAA to the cells in the presence of increasing concentrations of unlabeled SAA. The data was analyzed and plotted using LIGAND software on a Macintosh computer. Panel C shows the displacement of ¹²⁵I-SAA binding on monocytes by unlabeled SAA and fMLP. Same results were obtained with FPLR1/293 cells (panel D).

### DETAILED DESCRIPTION OF THE PREFERRED EMBODIMENT

In the disclosure herein, we describe the discovery of the SAA receptor on human phagocytes (FPRL1) and teach that modulation of the SAA/FPRL1 complex can influence cellular responses including, but not limited to, signal transduction, leukocyte migration, immune system response, amyloidosis, inflammatory response, infection, organ rejection, arthritis, atherosclerosis, and neoplasia. Novel biological tools for the study of the interaction of SAA, fragments of SAA, or homologs or derivatives thereof with FPRL1 are provided. Further, prophylactics, therapeutics, and methods of use of the foregoing, which modulate the association of SAA with FPRL1 and thereby effect the cellular responses described above, are taught.

We began the search that led to the discovery of the SAA/FPRL1 complex by investigating the effects of SAA and other chemoattractants on Ca²⁺ mobilization in monocytes and neutrophils. In primary cells, cross-desensitization of Ca²⁺ transients is often due to two agonists acting at the same receptor. (Wang et al., J. Exp. Med., 177:699-705 (1993)). During cross-desensitization experiments using SAA and other chemoattractants, we found that the bacterial chemotactic peptide N-formyl-methionyl-leucyl-phenylalanine (fMLP), when used at relatively high concentrations (10: M and more), was able to attenuate a subsequent cell response to SAA. These results led us to believe that SAA interacts with a low affinity fMLP receptor on leukocytes.

To date, two receptors that bind fMLP have been identified and cloned. (Reviewed in Prossnitz, E. R. and R. D. Ye, Pharmacol. Ther., 74:73-102 (1997); and Murphy, P. M., Chemoattractant ligands and their receptors, pp. 269 (1996)). The prototype receptor, FPR, binds fMLP with high affinity and is activated by low concentrations of fMLP. Another highly homologous variant of FPR, named FPRL1 (also referred to as FPRH2 and LXA4R), was originally cloned as an orphan receptor (Murphy et al., J. Biol. Chem., 267:7637-7643 (1992); Ye et al., Biochem. Biophys. Res. Commun., 184:582-589 (1992); Bao et al., Genomics, 13:437-440 (1992); Gao, J. L. and P. M. Murphy, J. Biol. Chem., 268:25395-25401 (1993); and Nomura et al., Int. Immunol., 5:1239-1249 (1993)) but was subsequently found to mediate Ca²⁺ mobilization in response to high concentrations of fMLP. (Ye et al., Biochem. Biophys. Res. Commun., 184:582-589 (1992); and Gao, J. L. and P. M. Murphy, J. Biol. Chem., 268:25395-25401 (1993)). Furthermore, a lipid metabolite, lipoxin A4 and its analogues, were found to bind FPRL1 with high affinity and increase arachidonic acid production and G-protein activation in FPRL1 transfected cells. (Fiore et al, J. Exp. Med., 180:253-260 (1994)).

LXA4 inhibits pro-inflammatory neutrophil responses (Fiore et al, J. Exp. Med., 180:253-260 (1994); Takano et al., J. Exp. Med., 185:1693-1704 (1997); Maddox et al., J. Biol. Chem., 272:6972-6978 (1997); Fiore et al., Blood, 81:3395-3403 (1993); Fiore, S. and C. N. Serhan, Biochemistry, 34:16678-16686 (1995); Gronert et al., J. Exp. Med., 187:1285-1294 (1998); Gewirtz et al., J. Clin. Invest., 101:1860-1869 (1998); Romano et al., J. Immunol., 157:2149-2154 (1994); and Colgan et al., J. Clin. Invest., 92:75-82 (1993)) as well as the release of the proinflammatory cytokine, IL-8 by epithelial cells. (Gronert et al., J. Exp. Med., 187:1285-1294 (1998); and Takano et al., J. Clin. Invest., 101:819-826 (1998)). These cellular responses have been attributed to activation of FPRL1 (or LXA4R) in neutrophils and epithelial cells.

Another lipid mediator receptor, the leukotriene B4 (LTB4) receptor, is structurally related to FPRL1 (30.7% amino acid sequence identity) (Yokomizo et al., Nature, 387:620-624 (1997)), and has also been reported to be a fusion co-factor for human immunodeficency virus type I (HIV-1) (Owman et al., Proc. Natl. Acad. Sci. USA., 95:9530-9534 (1998)), similar to various chemokine receptors (reviewed in ref. Berger, E. A., AIDS, 11:S3-16 (1997)). This activity has not been reported for FPRL1, however. (Murphy, P. M., Chemoattractant ligands and their receptors, pp. 269 (1996); and Berger, E. A., AIDS, 11 :S3-16 (1997)).

Because our cross de-sensitization experiments provided evidence that a low affinity fMLP receptor interacted with SAA, we performed chemotactic assays and radioactive SAA binding studies on cells that were transfected to express different receptors. In one experiment, for example, we selectively induced Ca²⁺ mobilization by administering SAA and observed the migration of cells that were transfected to express FPRL1 receptors, which were not endogenous to these cell types. In another experiment, we found that radiolabeled SAA bound to FPRL1 but not the FPR receptor. Through these investigations, we have discovered a novel interaction between FPRL1 and SAA.

By enhancing or inhibiting (“modulating”) assembly of the SAA/FPRL1 complex and/or SAA/FPRL1-mediated signal transduction, cellular responses such as signal transduction, leukocyte migration, immune system response, amyloidosis, inflammatory response, infection, organ rejection, arthritis, atherosclerosis, and neoplasia can be selectively altered. In some aspects of the invention, the modulation of the assembly of the SAA/FPRL1 complex is accomplished by using a modulator that is a nucleic acid embodiment. For example, a construct encoding FPRL1 is transfected into cells so as to raise the concentration of FPRL1 and thereby promote SAA/FPRL1 complex assembly or, alternatively, a construct encoding a nucleic acid that is complementary to a nucleic acid encoding FPRL1 (e.g., an antisense inhibitor or a ribozyme) is used to reduce the concentration of FPRL1 and thereby inhibit SAA/FPRL1 assembly. Further, in some embodiments, nucleic acids encoding wild-type or mutant SAA or fragments of SAA or complements of these nucleic acid molecules are transfected into cells so as to modulate SAA/FPRL1 complex assembly and/or SAA/FPRL1-mediated signal transduction.

According to other aspects, the modulation of the assembly of the SAA/FPRL1 complex and/or SAA/FPRL1-mediated signal transduction is achieved by using a modulator that is a protein-based embodiment. For example, FPRL1 or fragments thereof can be delivered to cells by liposome-mediated transfer or administered to cells exogenously so as to modulate assembly of the SAA/FPRL1 complex or, alternatively, wild-type or mutant SAA, or fragments thereof can be administered to cells so as to modulate assembly of the SAA/FPRL1 complex and/or SAA/FPRL1-mediated signal transduction. Peptidomimetics that resemble SAA or FPRL1 or fragments thereof are also modulators of the invention and can be used to effect assembly of the SAA/FPRL1 complex. Many chemicals can also be modulators and can be identified by their ability to effect assembly of the SAA/FPRL1 complex and/or SAA/FPRL1-mediated signal transduction using the SAA/FPRL1 characterization assays described below. These embodiments can be manufactured as monomeric, multimeric, and multimerized agents.

Several types of assays that provide information about SAA or FPRL1 or the formation of the SAA/FPRL1 complex are embodiments of the invention. These assays are collectively referred to as “SAA/FPRL1 characterization assays”. One type of SAA/FPRL1 characterization assay concerns measuring the ability of SAA or fragments thereof to bind to FPRL1 or vice versa. For example, methods of performing SAA/FPRL1 characterization assays are provided, in which FPRL1 or SAA are disposed on a support and are subsequently contacted with a ligand (e.g., SAA or FPRL1 or a fragment thereof, depending on the support-bound molecule) and assembly of the SAA/FPRL1 complex is determined. A similar binding assay can be employed in the presence of an inhibiting or enhancing molecule (a “modulator”) such as a peptide or peptidomimetic (collectively referred to as a “peptide agent”) or a chemical. The supports in these assays can be conventional resins, plastics, lipids, membranes, and cells.

Other aspects of the invention utilize software and hardware comprising nucleic acid sequences encoding SAA or FPRL1 or fragments thereof or complements of these sequences and protein sequences corresponding to SAA or FPRL1 or fragments thereof. Aspects of the invention also utilize software and hardware having the aforementioned sequences to characterize two dimensional and three dimensional aspects of SAA and FPRL1, which allows one to develop protein models of SAA and FPRL1, identify homologous proteins, and conduct rational drug design. Methods of identification of agents that modulate assembly of the SAA/FPRL1 complex and/or SAA/FPRL1-mediated signal transduction, for example, use software and hardware that have the aforementioned sequence information.

The embodied approaches to rational drug design seek to identify novel agents that interact with SAA or FPRL1 and modulate assembly of the SAA/FPRL1 complex and/or SAA/FPRL1-mediated signal transduction. Accordingly, protein models of SAA or FPRL1 or fragments of these molecules, and models of candidate modulating agents are constructed and combinatorial chemistry is employed to develop and identify agents that modulate assembly of the SAA/FPRL1 complex and/or SAA/FPRL1-mediated signal transduction. Once candidate agents are developed and identified, they are screened in a SAA/FPRL1 characterization assay (e.g., a SAA adhesion assay). The identity of each agent and its performance in a SAA/FPRL1 characterization assay, e.g., its effect on the modulation assembly of the SAA/FPRL1 complex, can be recorded on software or hardware and the recorded data can be used to create a library of SAA/FPRL1 modulating agents. These libraries can be employed to identify more agents that modulate assembly of the SAA/FPRL1 complex and are valuable clinical tools for manufacturing and selecting an appropriate pharmaceutical to treat a particular type of malady.

In the therapeutic and prophylactic embodiments of the invention, SAA or FPRL1, polypeptide fragments of SAA or FPRL1, and nucleic acids encoding these molecules, and agents that interact with a SAA/FPRL1 complex are incorporated into pharmaceuticals. These pharmaceuticals can be delivered by any conventional route including, but not limited to, transdermal, parenteral, gastrointestinal, transbronchial, and transalveolar. In addition to the active ingredients mentioned above, the pharmaceutical embodiments can comprise carriers, proteins, supports, adjuvants, or components that facilitate or enhance drug delivery. These pharmaceuticals can be employed in therapeutic protocols for the treatment and prevention of a SAA-related malady (e.g., an abnormal immune system response, amyloidosis, inflammation, infection, organ rejection, arthritis, atherosclerosis, and neoplasia). By one approach, a subject at risk for contracting a SAA-related malady or a subject already afflicted with a SAA-related malady is identified by conventional techniques and then is administered an effective amount of an agent that inhibits or promotes assembly of the SAA/FPRL1 complex. The discovery of the SAA/FPRL1 complex is disclosed in the following section.

Discovery of the SAA Receptor on Human Leukocytes

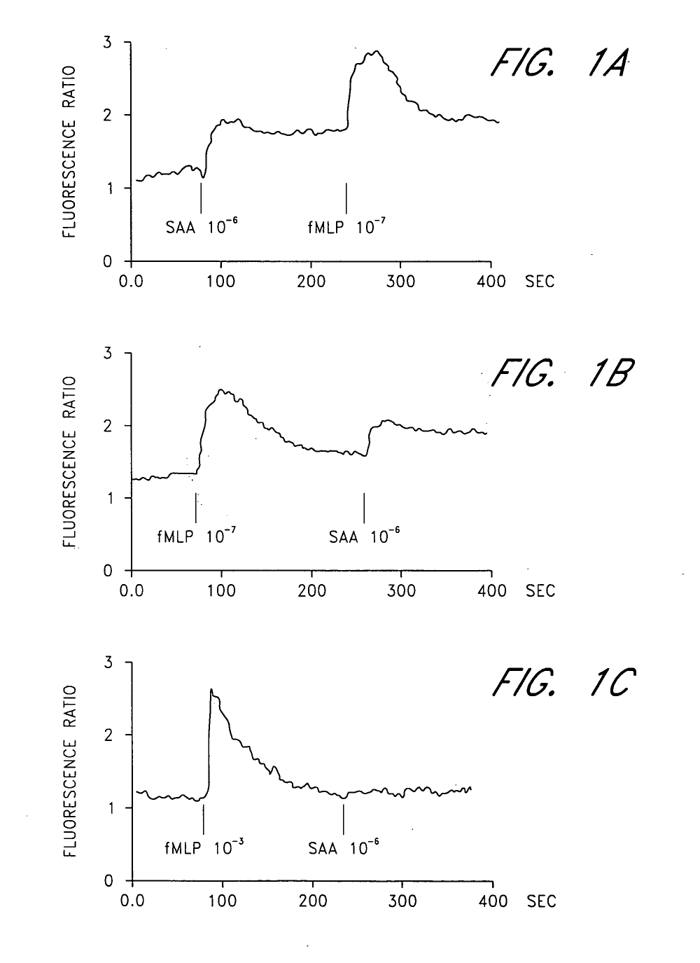

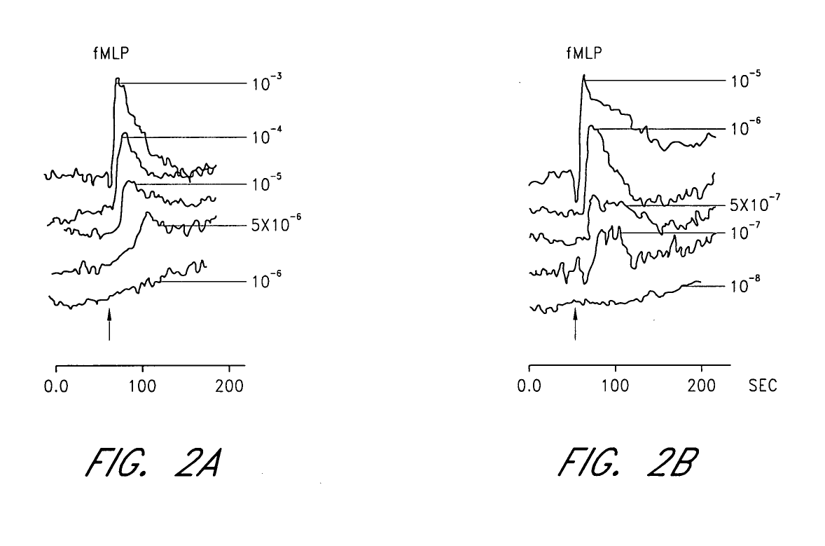

In a series of cross-desensitization experiments, we observed that SAA at 1: M failked to desensitize the Ca²⁺ flux in monocytes or neutrophils induced by chemokines such as MCP-1, RANTES, MCP-3, MIP-1∀ IL-8 and SDF-1∀. We concluded from these findings that SAA was unlikely to share a receptor with any of the chemokines tested. Additionally, we observed that SAA did not attenuate the cell response to the bacterial chemotactic N-formylated peptide fMLP, when fMLP was used at 100 nM (10⁻⁷ M) (FIG. 1A). In reciprocal tests, however, fMLP at 100 nM showed a partial desensitizing effect on SAA-induced Ca²⁺ mobilization in monocytes (FIG. 1B). The cellular response to SAA was completely desensitized by higher concentrations of fMLP (10⁻³ M=1 mM, FIG. 1C), providing evidence that SAA uses a low affinity fMLP receptor(s).

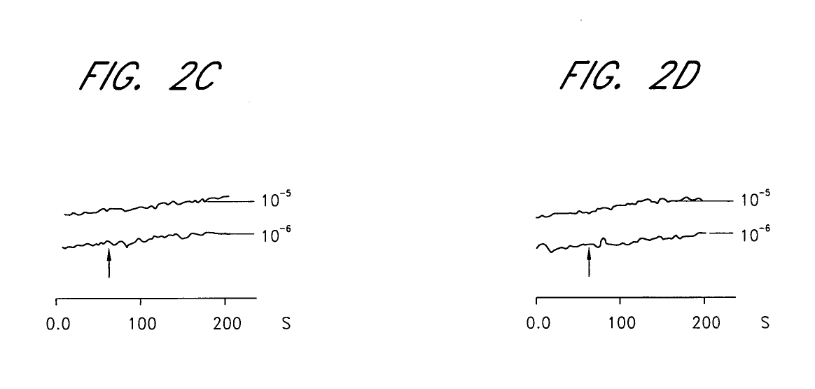

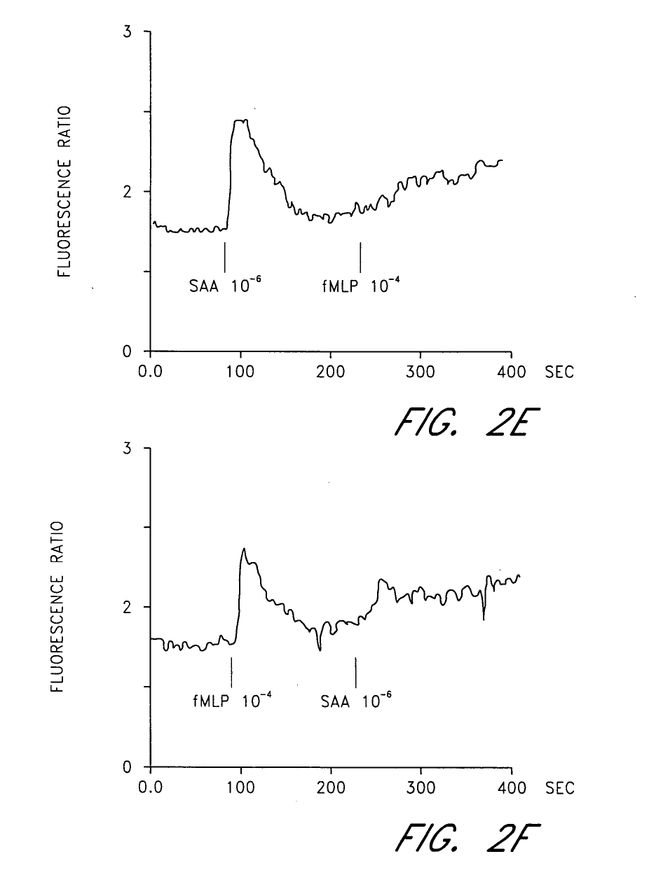

Since fMLP was known to induce Ca²⁺ mobilization in phagocytes through at least two seven transmembrane, G-protein-coupled receptors, FPR and FPRL1 (Murphy et al., J. Biol. Chem., 267:7637-7643 (1992); Ye et al., Biochem. Biophys. Res. Commun., 184:582-589 (1992); Gao, J. L. and P. M. Murphy, J. Biol. Chem., 268:25395-25401 (1993); and Murphy, P. M. and D. McDermott, J. Biol. Chem., 266:12560-12567 (1991)), we tested the effect of SAA using cells transfected to express these receptors. Prior to transfection, these cells were not responsive to fMLP stimulation. In the FPR-transfected rat basophil leukemia cell line (ETFR cells), we observed Ca²⁺ mobilization over a wide range of fMLP concentrations, with an EC50 of 10 pM. In contrast, the EC50 for fMLP to induce Ca²⁺ mobilization in FPRL1 transfected cells (FPRL1/293 cells) was much higher at 10: M (FIG. 2A). These results confirmed earlier observations that FPR is a high affinity receptor for fMLP, whereas FPRL1 has a much lower affinity. (Murphy et al., J. Biol. Chem., 267:7637-7643 (1992); Ye et al., Biochem. Biophys. Res. Commun., 184:582-589 (1992); Gao, J. L. and P. M. Murphy, J. Biol. Chem., 268:25395-25401 (1993); and Murphy, P. M. and D. McDermott, J. Biol. Chem., 266:12560-12567 (1991)).

Further, rhSAA induced Ca²⁺ mobilization in cells transfected with FPRL1 (FPRL1/293 cells) (FIG. 2B), but not in FPR expressing cells or mock transfected 293 cells (FIG. 2C, D). The EC50 of rhSAA on FPRL1 transfected cells was 250 nM, suggesting that SAA activates FPRL1 with higher efficacy than fMLP. This was supported by studies of cross-desensitization of Ca²⁺ flux between SAA and fMLP in FPRL1/293 cells. As shown in FIGS. 2E, F, although sequential stimulation of FPRL1/293 cells with SAA and fMLP resulted in bidirectional desensitization, SAA was able to desensitize the cell response to a 100 fold excess of fMLP. In contrast, fMLP at 100 fold excess of SAA, only partially desensitized the effect of SAA.

Leukocyte infiltration in vivo is believed to result from the migration of cells toward a gradient of locally produced chemoattractant(s). This process can be emulated by in vitro assays of chemotaxis, which provide a very sensitive and biologically relevant means of evaluating the function of cloned chemoattractant receptors. (Gong et al., J. Biol. Chem. 272:11682-11685 (1997); Wang et al., J. Exp. Med., 177:699-705 (1993); Gong et al., J. Biol. Chem., 272:11682-11685 (1997); and Ben-Baruch et al., J. Biol. Chem. 270:22123-22128 (1995)). Since SAA was known to induce leukocyte infiltration in vivo (Badolato et al., J. Exp. Med., 180:203-209 (1994); and Xu et al., J. Immunol., 155:1184-1190 (1995)) and chemotaxis in vitro (Badolato et al., J. Exp. Med., 180:203-209 (1994); and Xu et al., J. Immunol., 155:1184-1190 (1995)), we sought to determine whether SAA could induce cell migration via FPRL1.

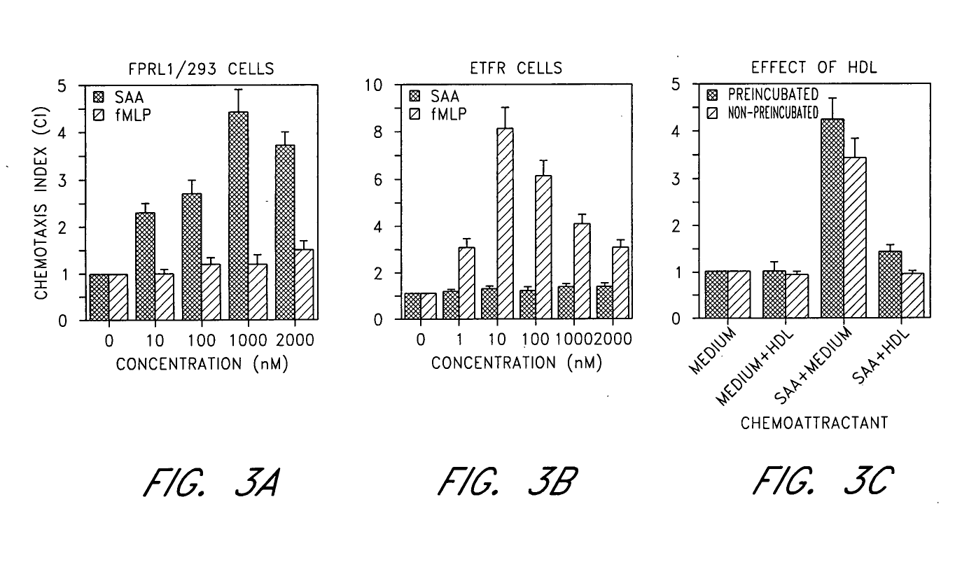

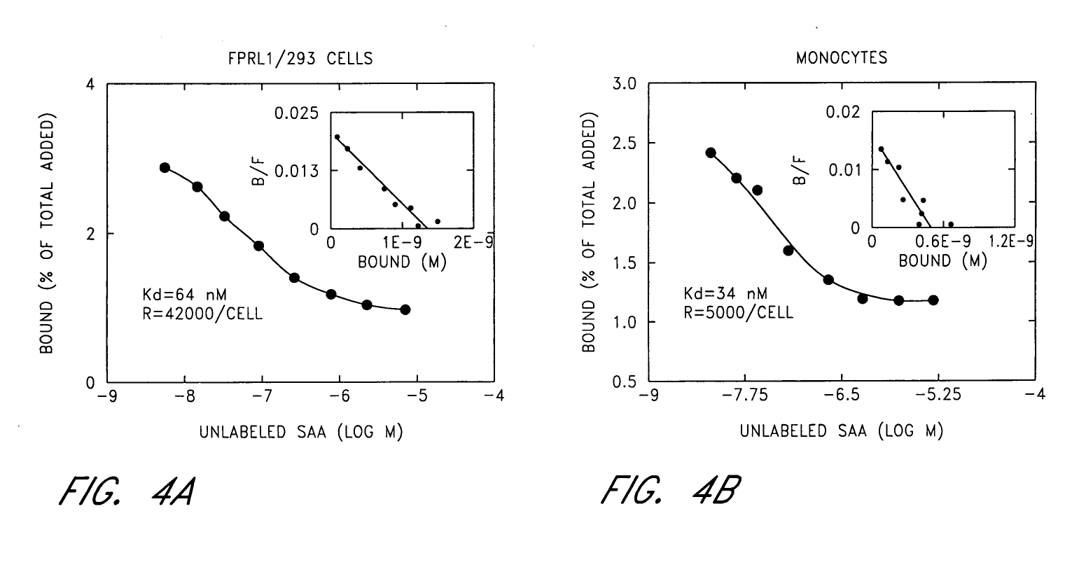

In our experiments on SAA induced chemotaxis in FPRL1/293 cells, we discovered that FPRL1/293 cells exhibit a potent migratory response to SAA, having an EC50 of 200 nM. Remarkably, these cells failed to migrate in response to a wide range of concentrations of fMLP (FIG. 3A). The fMLP induced migration of FPR expressing ETFR cells occurred at nM range concentrations, whereas the same cells did not migrate in response to SAA (FIG. 3B). These chemotaxis experiments established that fMLP is only a partial agonist for FPRL1 since the ligand failed to induce cell migration through FPRL1. On the other hand, SAA demonstrated full agonist activity on FPRL1. Further, both SAA induced Ca²⁺ mobilization and chemotaxis in FPRL1/293 cells were inhibited by pretreatment of the cells with pertussis toxin but not cholera toxin, which correlates with observations in native cells (Badolato et al., J. Exp. Med., 180:203-209 (1994); Xu et al., J. Immunol., 155:1184-1190 (1995); and Badolato et al., J. Immunol., 155:4004-4010 (1995)).

Additionally, we examined the effect of HDL on the chemotactic activity of SAA for FPRL1/293 cells. SAA can form complexes with HDL and HDL is a natural inhibitor of SAA. (Badolato et al., J. Exp. Med., 180:203-209 (1994); and Xu et al., J. Immunol., 155:1184-1190 (1995)). FIG. 3C shows that HDL, whether preincubated with SAA or simultaneously added to SAA, completely abolished SAA-induced FPRL1/293 cell migration. In contrast, the same concentration of HDL did not affect migration of FPR expressing ETFR cells induced by fMLP. These results confirmed that HDL specifically inhibited the agonist activity of SAA on FPRL1.

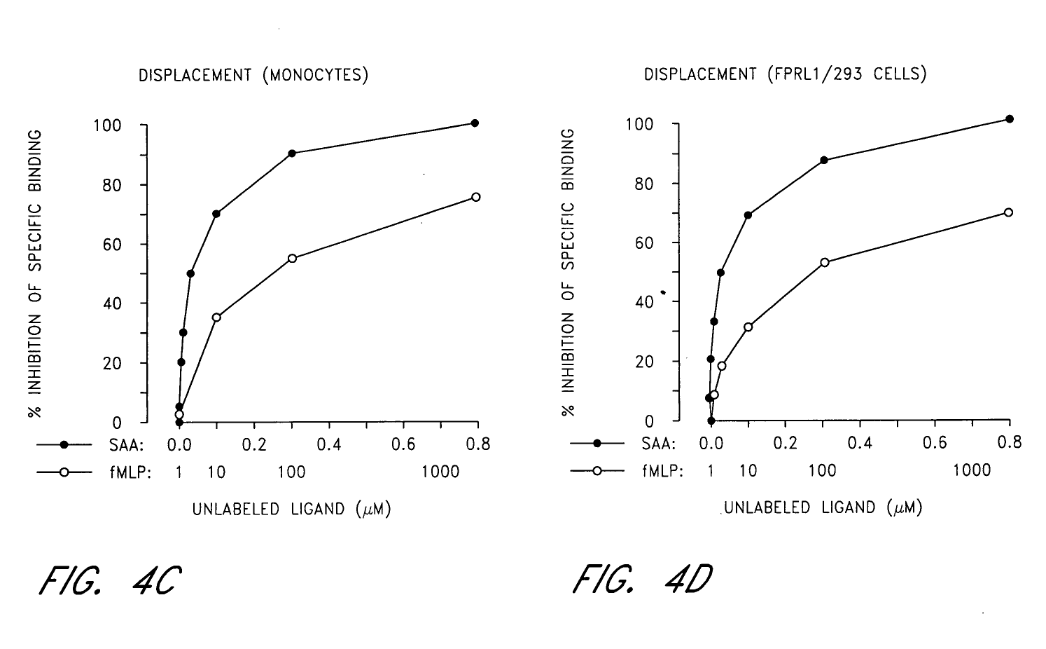

To further verify that FPRL1 is the SAA receptor, we performed ligand binding experiments. FIG. 4 shows that radioiodinated SAA specifically bound to FPRL1/293 cells with an estimated kd at 64 nM and 42000 binding sites/cell (FIG. 4A). ¹²⁵I-SAA also specifically bound to monocytes (FIG. 4B) and neutrophils (Kd=45 nM, R=6700/cell) with Kd values comparable to those achieved with FPRL1/293 cells. In the displacement assay, unlabeled SAA inhibited its own binding to monocytes (FIG. 4C), neutrophils and FPRL1/293 (FIG. 4D) in a dose-dependent manner, having an IC50 at about 50 nM. In contrast, unlabeled fMLP at high concentrations (≧10: M) only partially competed with ¹²⁵I-SAA for binding. These results demonstrated that SAA is a far more efficient agonist for FPRL1 than fMLP.

In the experiments presented above, we have established that SAA associates with FPRL1, a seven-transmembrane, G-protein coupled receptor that is expressed on phagocytes and promotes chemotaxis. (Badolato et al., J. Exp. Med., 180:203-209 (1994); Xu et al., J. Immunol., 155:1184-1190 (1995); and Badolato et al., J. Immunol., 155:4004-4010 (1995)). We have discovered that SAA mediates cellular responses by forming a complex with FPRL1. Although FPRL1 had previously been shown to be a low affinity receptor for fMLP (Ye et al., Biochem. Biophys. Res. Commun., 184:582-589 (1992); and Gao, J. L. and P. M. Murphy, J. Biol. Chem., 268:25395-25401 (1993)) and a high affinity receptor for lipid metabolite LXA4 and its analogues (Fiore et al, J. Exp. Med., 180:253-260 (1994); Takano et al., J. Exp. Med., 185:1693-1704 (1997); Maddox et al., J. Biol. Chem., 272:6972-6978 (1997); Fiore et al., Blood, 81:3395-3403 (1993); Fiore, S. and C. N. Serhan, Biochemistry, 34:16678-16686 (1995); Gronert et al., J. Exp. Med., 187:1285-1294 (1998); Gewirtz et al., J. Clin. Invest., 101:1860-1869 (1998); Romano et al., J. Immunol., 157:2149-2154 (1994); Colgan et al., J. Clin. Invest., 92:75-82 (1993); and Takano et al., J. Clin. Invest., 101:819-826 (1998)), until the present disclosure, it was not known that SAA interacts with FPRL1. Unlike fMLP, which is a partial agonist incapable of inducing chemotaxis via FPRL1, SAA is the first chemotactic agonist produced in humans identified for FPRL1.

FPRL1 was identified and molecularly cloned from human phagocytic cells by low stringency hybridization of the cDNA library with the FPR sequence and was initially defined as an orphan receptor. (Murphy et al., J. Biol. Chem., 267:7637-7643 (1992); Ye et al., Biochem. Biophys. Res. Commun., 184:582-589 (1992); Gao, J. L. and P. M. Murphy, J. Biol. Chem., 268:25395-25401 (1993); and Nomura et al., Int. Immunol., 5:1239-1249 (1993)). The cloning of the same receptor termed FPRH2 from a genomic library was described by Bao et al. (Bao et al., Genomics, 13:437-440 (1992)). FPRL1 possesses 69% identity at the amino acid level to FPR, the prototype receptor for synthetic and bacterium-derived formylated peptides (Prossnitz, E. R. and R. D. Ye, Pharmacol. Ther., 74:73-102 (1997); and Murphy, P. M., Chemoattractant ligands and their receptors, pp. 269 (1996)). Both FPR and FPRL1 are expressed by monocytes and neutrophils and are clustered on human chromosome 19q13. (Bao et al., Genomics, 13:437-440 (1992); and Durstin et al., Biochem. Biophys. Res. Commun., 201:174-179 (1994)). While fMLP is a high affinity agonist for FPR, it interacts with FPRL1 and transduces signals in response to fMLP only at high concentrations. (FIG. 2 and ref: Ye et al., Biochem. Biophys. Res. Commun., 184:582-589 (1992); Gao, J. L. and P. M. Murphy, J. Biol. Chem., 268:25395-25401 (1993); and Durstin et al., Biochem. Biophys. Res. Commun., 201:174-179 (1994)). SAA, on the other hand, selectively binds and activates FPRL1 at physiologically relevant concentrations, which under inflammatory stimulation could reach 80: M in the serum. (Sipe, J. D., Immunophysiology: The role of cells and cytokines in immunity and inflammation, 259-273 (1990); Kisilevsky, R., Medical Hypotheses, 35:337-341 (1991); Skinner, M., J. Inten. Med., 232:513-514 (1992); and Malle, E. and F. C. De Beer, Eur. J. Clin. Invest., 26:427-435 (1996)).

FPRL1 is predominantly expressed in monocytes and neutrophils. However, cells other than phagocytes such as hepatocytes have also been shown to express FPRL1. (Prossnitz, E. R. and R. D. Ye, Pharmacol. Ther., 74:73-102 (1997)). Recently, the expression of this receptor (also termed LXA4R) has been reported to be highly inducible in epithelial cells by specific cytokines. (Gronert et al., J. Exp. Med., 187:1285-1294 (1998)). Our previous study demonstrated that CD3⁺ human peripheral blood T lymphocytes were induced by SAA to migrate and adhere to endothelial cell monolayers. (Xu et al., J. Immunol., 155:1184-1190 (1995)). In fact, we detected specific binding sites for ¹²⁵I-SAA on human peripheral blood CD3⁺ T lymphocytes (Kd=300 nM, R=2200 sites/cell). Whether these binding sites on T lymphocytes represent FPRL1 or additional receptor(s) for SAA was not known until the present disclosure.

While the chemotactic formyl peptide fMLP has been shown to be a low efficiency agonist for FPRL1, a lipid metabolite lipoxin A4 (LXA4) has been reported to be a high affinity ligand and potent agonist for this receptor. (Fiore et al, J. Exp. Med., 180:253-260 (1994)). LXA₄ is an eicosanoid generated during a number of host reactions such as inflammation, thrombosis and atherosclerosis (Romano et al., J. Immunol., 157:2149-2154 (1994)), and was initially discovered as an inhibitor of immune response. (Reviewed in Samuelsson et al., Science, 237:1171-1176 (1987)). LXA4 was subquently reported to inhibit neutrophil chemotaxis (Lee et al., Biochem. Biophys. Res. Commun., 180:1416-1421 (1991)) and transepithelial migration induced by chemotactic agents. (Colgan et al., J. Clin. Invest., 92:75-82 (1993)). A seven transmembrane G-protein coupled receptor identical to FPRL1 was recently identified for LXA4. (Fiore et al, J. Exp. Med., 180:253-260 (1994); Takano et al., J. Exp. Med., 185:1693-1704 (1997); and Romano et al., J. Immunol., 157:2149-2154 (1994)). LXA4 bound with high affinity to CHO cells transfected with this receptor and increased GTPase activity and the release of esterified arachidonate. (Fiore et al, J. Exp. Med., 180:253-260 (1994)).

LXA4 has been proposed to be an endogenously produced ligand for FPRL1. (Fiore et al, J. Exp. Med., 180:253-260 (1994); and Takano et al., J. Exp. Med., 185:1693-1704 (1997)). Although LXA4 has not been reported to induce Ca²⁺ mobilization in neutrophils or FPRL1 transfected cells (Fiore et al, J. Exp. Med., 180:253-260 (1994)), it was shown to induce Ca²⁺ flux and chemotaxis in monocytes. (Maddox et al., J. Biol. Chem., 272:6972-6978 (1997); and Romano et al., J. Immunol., 157:2149-2154 (1994)). In our present investigations, we did not detect a significant induction of Ca²⁺ flux or chemotaxis in FPRL1/293 cells using commercially available LXA4 (Biomol, Plymouth Meeting, Pa.), nor did we observe inhibition of SAA signaling or binding by this LXA4 in either phagocytes or FPRL1/293 cells.

Our previous studies also demonstrated that both SAA-induced leukocyte chemotaxis and activation were inhibited by pertussis toxin. (Xu et al., J. Immunol., 155:1184-1190 (1995); and Badolato et al., J. Immunol., 155:4004-4010 (1995)). Furthermore, we have shown that signaling of SAA through FPRL1 was sensitive to pertussis toxin. Although the signal transduction pathways triggered by the interaction of SAA with FPRL1 are not yet fully understood, we believe that FPRL1 receptors share biochemical pathways with FPR. It is well known, for example, that binding of FPR by bacterium-derived or synthetic peptide agonists result in a G protein-mediated signaling cascade leading to phagocytic cell adhesion, chemotaxis, release of oxygen intermediates, enhanced phagocytosis and bacterial killing, as well as gene transcription. (Prossnitz, E. R. and R. D. Ye, Pharmacol. Ther., 74:73-102 (1997); and Murphy, P. M., Chemoattractant ligands and their receptors, pp. 269 (1996)). Activation of FPR by its agonists can also result in heterologous desensitization of the subsequent cell response to other G-protein receptor ligands including chemokines. (Ali et al., J. Biol. Chem., 268:24247-24254 (1993); and Ali et al., J. Biol. Chem., 271:3200-3206 (1996)). We believe that a similar “desensitizing” effect of receptor activation can be present with FPRL1.

For instance, SAA was initially reported as an inhibitor of neutrophil response to fMLP. (Linke et al., Biochem. Biophys. Res. Commun., 176:1100-1105 (1991)). In these experiments, neutrophils preincubated with SAA showed reduced superoxide release in response to fMLP. (Linke et al., Biochem. Biophys. Res. Commun., 176:1100-1105 (1991)). Our previous study also demonstrated that preincubation of monocytes and neutrophils with SAA reduced cell response to a number of chemoattractants including fMLP and chemokines. (Badolato et al., J. Immunol., 155:4004-4010 (1995)). Accordingly, we believe that FPRL1 is capable of transducing intracellular biochemical responses leading to desensitization of other G-protein coupled receptors.

The optimal concentrations for SAA to induce leukocyte migration, adhesion and tissue infiltration range from 0.8-4: M (Badolato et al., J. Exp. Med., 180:203-209 (1994); Xu et al., J. Immunol., 155:1184-1190 (1995); and Badolato et al., J. Immunol., 155:4004-4010 (1995)), which are higher than the SAA levels present in normal serum but well below the concentration seen during a systemic acute phase response. (Sipe, J. D., Immunophysiology: The role of cells and cytokines in immunity and inflammation, 259-273 (1990); Kisilevsky, R., Medical Hypotheses, 35:337-341 (1991); Skinner, M., J. Inten. Med., 232:513-514 (1992); and Malle, E. and F. C. De Beer, Eur. J. Clin. Invest., 26:427-435 (1996)). Increased serum levels of SAA have been observed in a number of inflammatory and infectious diseases as well as after organ transplantation. (Malle, E. and F. C. De Beer, Eur. J. Clin. Invest., 26:427-435 (1996)). The over-production of SAA by hepatocytes can also be induced by inflammatory stimuli such as LPS, IL-1, IL-6 and TNF. (Sipe, J. D., Immunophysiology: The role of cells and cytokines in immunity and inflammation, 259-273 (1990); Kisilevsky, R., Medical Hypotheses, 35:337-341 (1991); Skinner, M., J. Inten. Med., 232:513-514 (1992); and Malle, E. and F. C. De Beer, Eur. J. Clin. Invest., 26:427-435 (1996)). Additionally, macrophages have been reported to be an extra-hepatic source of SAA during inflammation (Steel et al., Scand. J. Immunol., 44:493-500 (1996)) and can produce relatively high concentractions in microcompartments. The SAA concentrations required for activating FPRL1 are well within the range in which native cells are activated as shown by the results presented herein

The expression of SAA mRNA in human atherosclerotic lesions and the induction of SAA by oxidized low-density lipoproteins also support our belief that SAA plays an important role in vascular injury and atherosclerosis. (Malle, E. and F. C. De Beer, Eur. J. Clin. Invest., 26:427-435 (1996)). Under normal conditions, most serum SAA will be associated with high density lipoprotein (HDL) which acts as a natural inhibitor of the chemotactic activity of SAA. (See Badolato et al., J. Exp. Med., 180:203-209 (1994); and Xu et al., J. Immunol., 155:1184-1190 (1995) and FIG. 3D). However, since SAA binds to HDL at equimolar ratios (Liang, J. and J. D. Sipe, J. Lipid Res., 36:37-46 (1995)), a rapid increase in concentration of locally produced SAA can establish a gradient of free active SAA with consequent recruitment of leukocytes into inflammatory sites. Desirably, an increase in concentration of SAA at local inflammatory sites can activate leukocytes to remove pathogenic agents. Furthermore, signals triggered by activated FPRL1 can result in unresponsiveness of leukocytes to additional stimulation, thus sequestering the cells, limiting the degree of inflammation, and providing therapeutic benefit. In the following section, several nucleic acid-based embodiments are provided.

Modulation of Assembly of the SAA/FPRL1 Complex Using a Nucleic Acid Agent

Full-length SAA and FPRL1 (nucleic acids are represented in italics) and fragments of these molecules that encode polypeptides that are involved in the assembly of the SAA/FPRL1 complex are embodiments of the invention. Further embodiments include nucleic acids that complement full-length SAA and FPRL1 and molecules that complement nucleic acids that encode polypeptides that are involved in the assembly of the SAA/FPRL1 complex. Desired embodiments include nucleic acids having at least 9 consecutive bases of SAA or FPRL1 or a sequence complementary thereto, wherein the nucleic acid encodes a polypeptide that is involved in the assembly of the SAA/FPRL1 complex or wherein the nucleic acid complements a nucleic acid that encodes a polypeptide that is involved in the assembly of the SAA/FPRL1 complex. Preferably, the nucleic acid embodiments comprise at least 12, 13, 14, 15, 16, 17, 18, or 19 consecutive nucleotides from SAA or FPRL1 or a nucleic acid that complements SAA or FPRL1, as conditions dictate. More preferably, the nucleic acid embodiments comprise at least 20-30 consecutive nucleotides from SAA or FPRL1 or a nucleic acid that complements SAA or FPRL1. In some cases, the nucleic acid embodiments comprise more than 30 nucleotides from the nucleic acids encoding SAA or FPRL1 or a nucleic acid that complements these molecules and in other cases, the nucleic acid embodiments comprise at least 40, at least 50, at least 75, at least 100, at least 150, or at least 200 consecutive nucleotides from the nucleic acids encoding SAA or FPRL1 or a nucleic acid that complements these molecules.

The nucleic acid embodiments described above can be altered by mutation, such as substitutions, additions, or deletions, which provide for sequences encoding functionally equivalent molecules. Due to the degeneracy of nucleotide coding sequences, other DNA sequences that encode substantially the same SAA or FPRL1 amino acid sequence that is involved in the assembly of an SAA/FPRL1 complex can be used. These include, but are not limited to, nucleic acid sequences comprising all or portions of SAA or FPRL1 or nucleic acids that complement all or part of SAA or FPRL1 that have been altered by the substitution of different codons that encode a functionally equivalent amino acid residue within the sequence, thus producing a silent change. Further, the nucleic acid embodiments of the invention can be used to modulate assembly of an SAA/FPRL1 complex and/or signal transduction (e.g., by upregulating or downregulating the expression of SAA or FPRL1) and, therefore, have several uses in biotechnological research and the treatment and prevention of disease. In the section below, several protein-based embodiments are provided.

Modulation of Assembly of the SAA/FPRL1 Complex Using a Peptide Agent

In several embodiments of the present invention, SAA and FPRL1 and/or fragments thereof are incorporated into biotechnological tools and pharmaceuticals for therapeutic and prophylactic application. Preferably, the SAA and FPRL1 peptides and/or fragments thereof correspond to sequences involved in assembly of the SAA/FPRL1 complex and/or SAA/FPRL1-mediated signal transduction. Desirable peptides are between three amino acids and 100 amino acids in length and have at least some portion of the sequence of a peptide that is involved in assembly of the SAA/FPRL1 complex and/or SAA/FPRL1-mediated signal transduction. Additionally, peptidomimetics that resemble SAA and FPRL1 and fragments thereof or peptides of between three and 100 amino acids having sequence involved in assembly of the SAA/FPRL1 complex and/or SAA/FPRL1-mediated signal transduction, are embodiments of the present invention. For example, an oligopeptide for use in aspects of the present invention can have three amino acids, four amino acids, five amino acids, six amino acids, seven amino acids, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty nine, or forty or fifty or sixty or seventy or eighty or ninety or one-hundred amino acids. Similarly, peptidomimetics of the present invention can have structures that resemble three amino acids, four amino acids, five amino acids, six amino acids, seven amino acids, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty nine, or forty or fifty or sixty or seventy or eighty or ninety or one-hundred amino acids.

Peptides for use in aspects of the present invention can also be modified, e.g., the peptides can have substituents not normally found on a peptide or the peptides can have substituents that are normally found on the peptide but are incorporated at regions of the peptide that are not normal. The peptides for use in aspects of the present invention can be acetylated, acylated, or aminated, for example. Substituents which can be included on the peptide so as to modify it include, but are not limited to, H, alkyl, aryl, alkenyl, alkynl, aromatic, ether, ester, unsubstituted or substituted amine, amide, halogen or unsubstituted or substituted sulfonyl or a 5 or 6 member aliphatic or aromatic ring. As used throughout this disclosure, the term “peptide agent” refers to a modified or unmodified peptide and a chemical or a peptidomimetic that structurally (three-dimensionally or two-dimensionally) resembles a modified or unmodified SAA or FPRL1 or a fragment of these molecules. Peptide agents also include ligands identified by the methods of rational drug design detailed below.

The SAA and FPRL1 peptides and fragments or derivatives thereof, include but are not limited to, those containing as a primary amino acid sequence all or part of the amino acid sequence of SAA and FPRL1 found in nature. Additionally, altered sequences in which functionally equivalent amino acid residues are substituted for residues within the sequence resulting in a silent change can also be present in the peptides of the invention. Accordingly, one or more amino acid residues within the sequence can be substituted by another amino acid of a similar polarity that acts as a functional equivalent, resulting in a silent alteration. Substitutes for an amino acid within the sequence can be selected from other members of the class to which the amino acid belongs. For example, the non-polar (hydrophobic) amino acids include alanine, leucine, isoleucine, valine, proline, phenylalanine, tryptophan, and methionine. The uncharged polar neutral amino acids include glycine, serine, threonine, cysteine, tyrosine, asparagine and glutamine. The positively charged (basic) amino acids include arginine, lysine, and histidine. The negatively charged (acidic) amino acids include aspartic acid and glutamic acid. The aromatic aminoacids include phenylalanine, tryptophan, and tyrosine. In other aspects of the invention, SAA and FPRL1 and fragments or derivatives thereof, which are differentially modified during or after translation, e.g., by phosphorylation, glycosylation, cross-linking, acylation, proteolytic cleavage, linkage to an antibody molecule, membrane molecule, or other ligand, are contemplated. (Ferguson et al., Ann. Rev. Biochem. 57:285-320 (1988)).

Many embodiments of the invention involve SAA or fragments of SAA or the use of these polypeptides or peptidomimetics that resemble the structures (collectively referred to as “peptide agents”). The polypeptide sequence of recombinant human SAA (rhSAA) is:

| | |
|---|---|
| MRSFFSFLGEAFDGARDMWRAYSDMREANYIGSDKYFHARGNYDAAKRGP | (SEQ. ID. No. 1) |
| GGVWAAEAISNARENIQRFFGRGAEDSLADQAANEWGRSGKDPNHFRPAGLPEKY. | |

Carboxy truncations of SAA include:

| | |
|---|---|
| MRS | |
| MRSF | (SEQ. ID. NO. 2) |
| MRSFF | (SEQ. ID. NO. 3) |
| MRSFFS | (SEQ. ID. NO. 4) |
| MRSFFSF | (SEQ. ID. NO. 5) |
| MRSFFSFL | (SEQ. ID. NO. 6) |
| MRSFFSFLG | (SEQ. ID. NO. 7) |
| MRSFFSFLGE | (SEQ. ID. NO. 8) |
| MRSFFSFLGEA | (SEQ. ID. NO. 9) |
| MRSFFSFLGEAF | (SEQ. ID. NO. 10) |
| MRSFFSFLGEAFD | (SEQ. ID. NO. 11) |
| MRSFFSFLGEAFDG | (SEQ. ID. NO. 12) |
| MRSFFSFLGEAFDGA | (SEQ. ID. NO. 13) |
| MRSFFSFLGEAFDGAR | (SEQ. ID. NO. 14) |
| MRSFFSFLGEAFDGARD | (SEQ. ID. NO. 15) |
| MRSFFSFLGEAFDGARDM | (SEQ. ID. NO. 16) |
| MRSFFSFLGEAFDGARDMW | (SEQ. ID. NO. 17) |
| MRSFFSFLGEAFDGARDMWR | (SEQ. ID. NO. 18) |
| MRSFFSFLGEAFDGARDMWRA | (SEQ. ID. NO. 19) |
| MRSFFSFLGEAFDGARDMWRAY | (SEQ. ID. NO. 20) |
| MRSFFSFLGEAFDGARDMWRAYS | (SEQ. ID. NO. 21) |
| MRSFFSFLGEAFDGARDMWRAYSD | (SEQ. ID. NO. 22) |
| MRSFFSFLGEAFDGARDMWRAYSDM | (SEQ. ID. NO. 23) |
| MRSFFSFLGEAFDGARDMWRAYSDMR | (SEQ. ID. NO. 24) |
| MRSFFSFLGEAFDGARDMWRAYSDMRE | (SEQ. ID. NO. 25) |
| MRSFFSFLGEAFDGARDMWRAYSDMREA | (SEQ. ID. NO. 26) |
| MRSFFSFLGEAFDGARDMWRAYSDMREAN | (SEQ. ID. NO. 27) |
| MRSFFSFLGEAFDGARDMWRAYSDMREANY | (SEQ. ID. NO. 28) |
| MRSFFSFLGEAFDGARDMWRAYSDMREANYI | (SEQ. ID. NO. 29) |
| MRSFFSFLGEAFDGARDMWRAYSDMREANYIG | (SEQ. ID. NO. 30) |
| MRSFFSFLGEAFDGARDMWRAYSDMREANYIGS | (SEQ. ID. NO. 31) |
| MRSFFSFLGEAFDGARDMWRAYSDMREANYIGSD | (SEQ. ID. NO. 32) |
| MRSFFSFLGEAFDGARDMWRAYSDMREANYIGSDK | (SEQ. ID. NO. 33) |
| MRSFFSFLGEAFDGARDMWRAYSDMREANYIGSDKY | (SEQ. ID. NO. 34) |
| MRSFFSFLGEAFDGARDMWRAYSDMREANYIGSDKYF | (SEQ. ID. NO. 35) |
| MRSFFSFLGEAFDGARDMWRAYSDMREANYIGSDKYFH | (SEQ. ID. NO. 36) |
| MRSFFSFLGEAFDGARDMWRAYSDMREANYIGSDKYFHA | (SEQ. ID. NO. 37) |
| MRSFFSFLGEAFDGARDMWRAYSDMREANYIGSDKYFHAR | (SEQ. ID. NO. 38) |
| MRSFFSFLGEAFDGARDMWRAYSDMREANYIGSDKYFHARG | (SEQ. ID. NO. 39) |
| MRSFFSFLGEAFDGARDMWRAYSDMREANYIGSDKYFHARGN | (SEQ. ID. NO. 40) |
| MRSFFSFLGEAFDGARDMWRAYSDMREANYIGSDKYFHARGNY | (SEQ. ID. NO. 41) |
| MRSFFSFLGEAFDGARDMWRAYSDMREANYIGSDKYFHARGNYD | (SEQ. ID. NO. 42) |
| MRSFFSFLGEAFDGARDMWRAYSDMREANYIGSDKYFHARGNYDA | (SEQ. ID. NO. 43) |
| MRSFFSFLGEAFDGARDMWRAYSDMREANYIGSDKYFHARGNYDAA | (SEQ. ID. NO. 44) |
| MRSFFSFLGEAFDGARDMWRAYSDMREANYIGSDKYFHARGNYDAAK | (SEQ. ID. NO. 45) |
| MRSFFSFLGEAFDGARDMWRAYSDMREANYIGSDKYFHARGNYD | (SEQ. ID. NO. 46) |
| AAKR | |
| MRSFFSFLGEAFDGARDMWRAYSDMREANYIGSDKYFHARGNYD | (SEQ. ID. NO. 47) |
| AAKRG | |
| MRSFFSFLGEAFDGARDMWRAYSDMREANYIGSDKYFHARGNYD | (SEQ. ID. NO. 48) |
| AAKRGP | |
| MRSFFSFLGEAFDGARDMWRAYSDMREANYIGSDKYFHARGNYD | (SEQ. ID. NO. 49) |
| AAKRGPG | |
| MRSFFSFLGEAFDGARDMWRAYSDMREANYIGSDKYFHARGNYD | (SEQ. ID. NO. 50) |
| AAKRGPGG | |
| MRSFFSFLGEAFDGARDMWRAYSDMREANYIGSDKYFHARGNYD | (SEQ. ID. NO. 51) |
| AAKRGPGGV | |
| MRSFFSFLGEAFDGARDMWRAYSDMREANYIGSDKYFHARGNYD | (SEQ. ID. NO. 52) |
| AAKRGPGGVW | |
| MRSFFSFLGEAFDGARDMWRAYSDMREANYIGSDKYFHARGNYD | (SEQ. ID. NO. 53) |
| AAKRGPGGVWA | |
| MRSFFSFLGEAFDGARDMWRAYSDMREANYLGSDKYFHARGNYD | (SEQ. ID. NO. 54) |
| AAKRGPGGVWAA | |
| MRSFFSFLGEAFDGARDMWRAYSDMREANYIGSDKYFHARGNYD | (SEQ. ID. NO. 55) |
| AAKRGPGGVWAAE | |
| MRSFFSFLGEAFDGARDMWRAYSDMREANYIGSDKYFHARGNYD | (SEQ. ID. NO. 56) |
| AAKRGPGGVWAAEA | |
| MRSFFSFLGEAFDGARDMWRAYSDMREANYIGSDKYFHARGNYD | (SEQ. ID. NO. 57) |
| AAKRGPGGVWAAEAI | |
| MRSFFSFLGEAFDGARDMWRAYSDMREANYIGSDKYFHARGNYD | (SEQ. ID. NO. 58) |
| AAKRGPGGVWAAEAIS | |
| MRSFFSFLGEAFDGARDMWRAYSDMREANYIGSDKYFHARGNYD | (SEQ. ID. NO. 59) |
| AAKRGPGGVWAAEAISN | |
| MRSFFSFLGEAFDGARDMWRAYSDMREANYIGSDKYFHARGNYD | (SEQ. ID. NO. 60) |
| AAKRGPGGVWAAEAISNA | |
| MRSFFSFLGEAFDGARDMWRAYSDMREANYIGSDKYFHARGNYD | (SEQ. ID. NO. 61) |
| AAKRGPGGVWAAEAISNAR | |
| MRSFFSFLGEAFDGARDMWRAYSDMREANYIGSDKYFHARGNYD | (SEQ. ID. NO. 62) |
| AAKRGPGGVWAAEAISNARE | |
| MRSFFSFLGEAFDGARDMWRAYSDMREANYIGSDKYFHARGNYD | (SEQ. ID. NO. 63) |
| AAKRGPGGVWAAEAISNARENI | |
| MRSFFSFLGEAFDGARDMWRAYSDMREANYIGSDKYFHARGNYD | (SEQ. ID. NO. 64) |
| AAKRGPGGVWAAEAISNARENIQ | |
| MRSFFSFLGEAFDGARDMWRAYSDMREANYIGSDKYFHARGNYD | (SEQ. ID. NO. 65) |
| AAKRGPGGVWAAEAISNARENIQR | |
| MRSFFSFLGEAFDGARDMWRAYSDMREANYIGSDKYFHARGNYD | (SEQ. ID. NO. 66) |
| AAKRGPGGVWAAEAISNARENIQRF | |
| MRSFFSFLGEAFDGARDMWRAYSDMREANYIGSDKYFHARGNYD | (SEQ. ID. NO. 67) |
| AAKRGPGGVWAAEAISNARENIQRFF | |
| MRSFFSFLGEAFDGARDMWRAYSDMREANYIGSDKYFHARGNYD | (SEQ. ID. NO. 68) |
| AAKRGPGGVWAAEAISNARENIQRFFG | |
| MRSFFSFLGEAFDGARDMWRAYSDMREANYIGSDKYFHARGNYD | (SEQ. ID. NO. 69) |
| AAKRGPGGVWAAEAISNARENIQRFFGR | |
| MRSFFSFLGEAFDGARDMWRAYSDMREANYIGSDKYFHARGNYD | (SEQ. ID. NO. 70) |
| AAKRGPGGVWAAEAISNARENIQRFFGRG | |
| MRSFFSFLGEAFDGARDMWRAYSDMREANYIGSDKYFHARGNYD | (SEQ. ID. NO. 71) |
| AAKRGPGGVWAAEAISNARENIQRFFGRGA | |
| MRSFFSFLGEAFDGARDMWRAYSDMREANYIGSDKYFHARGNYD | (SEQ. ID. NO. 72) |
| AAKRGPGGVWAAEAISNARENIQRFFGRGAE | |
| MRSFFSFLGEAFDGARDMWRAYSDMREANYIGSDKYFHARGNYD | (SEQ. ID. NO. 73) |
| AAKRGPGGVWAAEAISNARENIQRFFGRGAED | |
| MRSFFSFLGEAFDGARDMWRAYSDMREANYIGSDKYFHARGNYD | (SEQ. ID. NO. 74) |
| AAKRGPGGVWAAEAISNARENIQRFFGRGAEDS | |
| MRSFFSFLGEAFDGARDMWRAYSDMREANYIGSDKYFHARGNYD | (SEQ. ID. NO. 75) |
| AAKRGPGGVWAAEAISNARENIQRFFGRGAEDSL | |
| MRSFFSFLGEAFDGARDMWRAYSDMREANYIGSDKYFHARGNYD | (SEQ. ID. NO. 76) |
| AAKRGPGGVWAAEAISNARENIQRFFGRGAEDSLA | |
| MRSFFSFLGEAFDGARDMWRAYSDMREANYIGSDKYFHARGNYD | (SEQ. ID. NO. 77) |
| AAKRGPGGVWAAEAISNARENIQRFFGRGAEDSLAD | |
| MRSFFSFLGEAFDGARDMWRAYSDMREANYIGSDKYFHARGNYD | (SEQ. ID. NO. 78) |
| AAKRGPGGVWAAEAISNARENIQRFFGRGAEDSLADQ | |
| MRSFFSFLGEAFDGARDMWRAYSDMREANYIGSDKYFHARGNYD | (SEQ. ID. NO. 79) |
| AAKRGPGGVWAAEAISNARENIQRFFGRGAEDSLADQA | |
| MRSFFSFLGEAFDGARDMWRAYSDMREANYIGSDKYFHARGNYD | (SEQ. ID. NO. 80) |
| AAKRGPGGVWAAEAISNARENIQRFFGRGAEDSLADQAA | |
| MRSFFSFLGEAFDGARDMWRAYSDMREANYIGSDKYFHARGNYD | (SEQ. ID. NO. 81) |
| AAKRGPGGVWAAEAISNARENIQRFFGRGAEDSLADQAAN | |
| MRSFFSFLGEAFDGARDMWRAYSDMREANYIGSDKYFHARGNYD | (SEQ. ID. NO. 82) |
| AAKRGPGGVWAAEAISNARENIQRFFGRGAEDSLADQAANE | |
| MRSFFSFLGEAFDGARDMWRAYSDMREANYIGSDKYFHARGNYD | (SEQ. ID. NO. 83) |
| AAKRGPGGVWAAEAISNARENIQRFFGRGAEDSLADQAANEW | |
| MRSFFSFLGEAFDGARDMWRAYSDMREANYIGSDKYFHARGNYD | (SEQ. ID. NO. 84) |
| AAKRGPGGVWAAEAISNARENIQRFFGRGAEDSLADQAANEWG | |
| MRSFFSFLGEAFDGARDMWRAYSDMREANYIGSDKYFHARGNYD | (SEQ. ID. NO. 85) |
| AAKRGPGGVWAAEAISNARENIQRFFGRGAEDSLADQAANEWGR | |
| MRSFFSFLGEAFDGARDMWRAYSDMREANYIGSDKYFHARGNYD | (SEQ. ID. NO. 86) |
| AAKRGPGGVWAAEAISNARENIQRFFGRGAEDSLADQAANEWGRS | |
| MRSFFSFLGEAFDGARDMWRAYSDMREANYIGSDKYFHARGNYD | (SEQ. ID. NO. 87) |
| AAKRGPGGVWAAEAISNARENIQRFFGRGAEDSLADQAANEWGRSG | |
| MRSFFSFLGEAFDGARDMWRAYSDMREANYIGSDKYFHARGNYD | (SEQ. ID. NO. 88) |
| AAKRGPGGVWAAEAISNARENIQRFFGRGAEDSLADQAANEWGRSGK | |
| MRSFFSFLGEAFDGARDMWRAYSDMREANYIGSDKYFHARGNYD | (SEQ. ID. NO. 89) |
| AAKRGPGGVWAAEAISNARENIQRFFGRGAEDSLADQAANEWGRSGKD | |
| MRSFFSFLGEAFDGARDMWRAYSDMREANYIGSDKYFHARGNYD | (SEQ. ID. NO. 90) |
| AAKRGPGGVWAAEAISNARENIQRFFGRGAEDSLADQAANEWGRSGKDP | |
| MRSFFSFLGEAFDGARDMWRAYSDMREANYIGSDKYFHARGNYD | (SEQ. ID. NO. 91) |
| AAKRGPGGVWAAEAISNARENIQRFFGRGAEDSLADQAANEWGR | |
| SGKDPN | |
| MRSFFSFLGEAFDGARDMWRAYSDMREANYIGSDKYFHARGNYD | (SEQ. ID. NO. 92) |
| AAKRGPGGVWAAEAISNARENIQRFFGRGAEDSLADQAANEWGR | |
| SGKDPNH | |
| MRSFFSFLGEAFDGARDMWRAYSDMREANYIGSDKYFHARGNYD | (SEQ. ID. NO. 93) |
| AAKRGPGGVWAAEAISNARENIQRFFGRGAEDSLADQAANEWGR | |
| SGKDPNHF | |
| MRSFFSFLGEAFDGARDMWRAYSDMREANYIGSDKYFHARGNYD | (SEQ. ID. NO. 94) |
| AAKRGPGGVWAAEAISNARENIQRFFGRGAEDSLADQAANEWGR | |
| SGKDPNHFR | |
| MRSFFSFLGEAFDGARDMWRAYSDMREANYIGSDKYFHARGNYD | (SEQ. ID. NO. 95) |
| AAKRGPGGVWAAEAISNARENIQRFFGRGAEDSLADQAANEWGR | |
| SGKDPNHFRP | |
| MRSFFSFLGEAFDGARDMWRAYSDMREANYIGSDKYFHARGNYD | (SEQ. ID. NO. 96) |
| AAKRGPGGVWAAEAISNARENIQRFFGRGAEDSLADQAANEWGR | |
| SGKDPNHFRPA | |
| MRSFFSFLGEAFDGARDMWRAYSDMREANYIGSDKYFHARGNYD | (SEQ. ID. NO. 97) |
| AAKRGPGGVWAAEAISNARENIQRFFGRGAEDSLADQAANEWGR | |
| SGKDPNHFRPAG | |
| MRSFFSFLGEAFDGARDMWRAYSDMREANYIGSDKYFHARGNYD | (SEQ. ID. NO. 98) |
| AAKRGPGGVWAAEAISNARENIQRFFGRGAEDSLADQAANEWGR | |
| SGKDPNHFRPAGL | |
| MRSFFSFLGEAFDGARDMWRAYSDMREANYIGSDKYFHARGNYD | (SEQ. ID. NO. 99) |
| AAKRGPGGVWAAEAISNARENIQRFFGRGAEDSLADQAANEWGR | |
| SGKDPNHFRPAGLP | |
| MRSFFSFLGEAFDGARDMWRAYSDMREANYIGSDKYFHARGNYD | (SEQ. ID. NO. 100) |
| AAKRGPGGVWAAEAISNARENIQRFFGRGAEDSLADQAANEWGR | |
| SGKDPNHFRPAGLPE | |
| MRSFFSFLGEAFDGARDMWRAYSDMREANYIGSDKYFHARGNYD | (SEQ. ID. NO. 101) |
| AAKRGPGGVWAAEAISNARENIQRFFGRGAEDSLADQAANEWGR | |
| SGKLDPNHFRPAGLPEK | |

Amino truncations of SAA include:

| | |
|---|---|
| RSFFSFLGEAFDGARDMWRAYSDMREANYIGSDKYFHARGNYDA | (SEQ. ID. NO. 102) |
| AKRGPGGVWAAEAISNARENIQRFFGRGAEDSLADQAANEWGRS | |
| GKDPNHFRPAGLPEKY | |
| SFFSFLGEAFDGARDMWRAYSDMREANYIGSDKYFHARGNYDAA | (SEQ. ID. NO. 103) |
| KRGPGGVWAAEAISNARENIQRFFGRGAEDSLADQAANEWGRSGK | |
| DPNHFRPAGLPEKY | |
| FFSFLGEAFDGARDMWRAYSDMREANYIGSDKYFHARGNYDAAK | (SEQ. ID. NO. 104) |
| RGPGGVWAAEAISNARENIQRFFGRGAEDSLADQAANEWGRSGKD | |
| PNHFRPAGLPEKY | |
| FSFLGEAFDGARDMWRAYSDMREANYIGSDKYFHARGNYDAAK | (SEQ. ID. NO. 105) |
| RGPGGVWAAEAISNARENIQRFFGRGAEDSLADQAANEWGRSGK | |
| DPNHFRPAGLPEKY | |
| SFLGEAFDGARDMWRAYSDMREANYIGSDKYFHARGNYDAAKR | (SEQ. ID. NO. 106) |
| GPGGVWAAEAISNARENIQRFFGRGAEDSLADQAANEWGRSGK | |
| DPNHFRPAGLPEKY | |
| FLGEAFDGARDMWRAYSDMREANYIGSDKYFHARGNYDAAKRG | (SEQ. ID. NO. 107) |
| PGGVWAAEAISNARENIQRFFGRGAEDSLADQAANEWGRSGKD | |
| PNHFRPAGLPEKY | |
| LGEAFDGARDMWRAYSDMREANYIGSDKYFHARGNYDAAKRGP | (SEQ. ID. NO. 108) |
| GGVWAAEAISNARENIQRFFGRGAEDSLADQAANEWGRSGKDP | |
| NHFRPAGLPEKY | |
| GEAFDGARDMWRAYSDMREANYIGSDKYFHARGNYDAAKRGPG | (SEQ. ID. NO. 109) |
| GVWAAEAISNARENIQRFFGRGAEDSLADQAANEWGRSGKDPN | |
| HFRPAGLPEKY | |
| EAFDGARDMWRAYSDMREANYIGSDKYFHARGNYDAAKRGPGG | (SEQ. ID. NO. 110) |
| VWAAEAISNARENIQRFFGRGAEDSLADQAANEWGRSGKDPNH | |
| FRPAGLPEKY | |
| AFDGARDMWRAYSDMREANYIGSDKYFHARGNYDAAKRGPGGV | (SEQ. ID. NO. 111) |
| WAAEAISNARENIQRFFGRGAEDSLADQAANEWGRSGKDPNHF | |
| RPAGLPEKY | |
| FDGARDMWRAYSDMREANYIGSDKYFHARGNYDAAKRGPGGV | (SEQ ID. NO. 112) |
| WAAEAISNARENIQRFFGRGAEDSLADQAANEWGRSGKDPNHF | |
| RPAGLPEKY | |
| DGARDMWRAYSDMREANYIGSDKYFHARGNYDAAKRGPGGVW | (SEQ. ID. NO. 113) |
| AAEAISNARENIQRFFGRGAEDSLADQAANEWGRSGKDPNHFR | |
| PAGLPEKY | |
| GARDMWRAYSDMREANYIGSDKYFHARGNYDAAKRGPGGVWA | (SEQ. ID. NO. 114) |
| AEAISNARENIQRFFGRGAEDSLADQAANEWGRSGKDPNHFR | |
| PAGLPEKY | |
| ARDMWRAYSDMREANYIGSDKYFHARGNYDAAKRGPGGVWAA | (SEQ. ID. NO. 115) |
| EAISNARENIQRFFGRGAEDSLADQAANEWGRSGKDPNHFRP | |
| AGLPEKY | |
| RDMWRAYSDMREANYIGSDKYFHARGNYDAAKRGPGGVWAAE | (SEQ. ID. NO. 116) |
| AISNARENIQRFFGRGAEDSLADQAANEWGRSGKDPNHFRPA | |
| GLPEKY | |
| DMWRAYSDMREANYIGSDKYFHARGNYDAAKRGPGGVWAAEAI | (SEQ. ID. NO. 117) |
| SNARENIQRFFGRGAEDSLADQAANEWGRSGKDPNHFRPAGLPEKY | |
| MWRAYSDMREANYIGSDKYFHARGNYDAAKRGPGGVWAAEAIS | (SEQ. ID. NO. 118) |
| NARENIQRFFGRGAEDSLADQAANEWGRSGKDPNHFRPAGLPEKY | |
| WRAYSDMREANYIGSDKYFHARGNYDAAKRGPGGVWAAEAISN | (SEQ. ID. NO. 119) |
| ARENIQRFFGRGAEDSLADQAANEWGRSGKDPNHFRPAGLPEKY | |
| RAYSDMREANYIGSDKYFHARGNYDAAKRGPGGVWAAEAISNAR | (SEQ. ID. NO. 120) |
| ENIQRFFGRGAEDSLADQAANEWGRSGKDPNHFRPAGLPEKY | |
| YSDMREANYIGSDKYFHARGNYDAAKRGPGGVWAAEAISNAREN | (SEQ. ID. NO. 121) |
| IQRFFGRGAEDSLADQAANEWGRSGKDPNHFRPAGLPEKY | |
| SDMREANYIGSDKYFHARGNYDAAKRGPGGVWAAEAISNARENI | (SEQ. ID. NO. 122) |
| QRFFGRGAEDSLADQAANEWGRSGKDPNHFRPAGLPEKY | |
| DMREANYIGSDKYFHARGNYDAAKRGPGGVWAAEAISNARENIQ | (SEQ. ID. NO. 123) |
| RFFGRGAEDSLADQAANEWGRSGKDPNHFRPAGLPEKY | |
| MREANYIGSDKYFHARGNYDAAKRGPGGVWAAEAISNARENIQR | (SEQ. ID. NO. 124) |
| FFGRGAEDSLADQAANEWGRSGKDPNHFRPAGLPEKY | |
| REANYIGSDKYFHARGNYDAAKRGPGGVWAAEAISNARENIQRFF | (SEQ. ID. NO. 125) |
| GRGAEDSLADQAANEWGRSGKDPNHFRPAGLPEKY | |
| EANYIGSDKYFHARGNYDAAKRGPGGVWAAEAISNARENIQRFFG | (SEQ. ID. NO. 126) |
| RGAEDSLADQAANEWGRSGKDPNHFRPAGLPEKY | |
| ANYIGSDKYFHARGNYDAAKRGPGGVWAAEAISNARENIQRFFGR | (SEQ. ID. NO. 127) |
| GAEDSLADQAANEWGRSGKDPNHFRPAGLPEKY | |
| NYIGSDKYFHARGNYDAAKRGPGGVWAAEAISNARENIQRFFGRG | (SEQ. ID. NO. 128) |
| AEDSLADQAANEWGRSGKDPNHFRPAGLPEKY | |
| YIGSDKYFHARGNYDAAKRGPGGVWAAEAISNARENIQRFFGRGA | (SEQ. ID. NO. 129) |
| EDSLADQAANEWGRSGKDPNHFRPAGLPEKY | |
| IGSDKYFHARGNYDAAKRGPGGVWAAEAISNARENIQRFFGRGAE | (SEQ. ID. NO. 130) |
| DSLADQAANEWGRSGKDPNHFRPAGLPEKY | |
| GSDKYFHARGNYDAAKRGPGGVWAAEAISNARENIQRFFGRGAE | (SEQ. ID. NO. 131) |
| DSLADQAANEWGRSGKDPNHFRPAGLPEKY | |
| SDKYFHARGNYDAAKRGPGGVWAAEAISNARENIQRFFGRGAED | (SEQ. ID. NO. 132) |
| SLADQAANEWGRSGKDPNHFRPAGLPEKY | |
| DKYFHARGNYDAAKRGPGGVWAAEAISNARENIQRFFGRGAEDS | (SEQ. ID. NO. 133) |
| LADQAANEWGRSGKDPNHFRPAGLPEKY | |
| KYFHARGNYDAAKRGPGGVWAAEAISNARENIQRFFGRGAEDSL | (SEQ. ID. NO. 134) |
| ADQAANEWGRSGKDPNHFRPAGLPEKY | |
| YFHARGNYDAAKRGPGGVWAAEAISNARENIQRFFGRGAEDSLA | (SEQ. ID. NO. 135) |
| DQAANEWGRSGKDPNHFRPAGLPEKY | |
| FHARGNYDAAKRGPGGVWAAEAISNARENIQRFFGRGAEDSLAD | (SEQ. ID. NO. 136) |
| QAANEWGRSGKDPNHFRPAGLPEKY | |
| HARGNYDAAKRGPGGVWAAEAISNARENIQRFFGRGAEDSLADQ | (SEQ. ID. NO. 137) |
| AANEWGRSGKDPNHFRPAGLPEKY | |
| ARGNYDAAKRGPGGVWAAEAISNARENIQRFFGRGAEDSLADQA | (SEQ. ID. NO. 138) |
| ANEWGRSGKDPNHFRPAGLPEKY | |
| RGNYDAAKRGPGGVWAAEAISNARENIQRFFGRGAEDSLADQAA | (SEQ. ID. NO. 139) |
| NEWGRSGKDPNHFRPAGLPEKY | |
| GNYDAAKRGPGGVWAAEAISNARENIQRFFGRGAEDSLADQAAN | (SEQ. ID. NO. 140) |
| EWGRSGKDPNHFRPAGLPEKY | |
| NYDAAKRGPGGVWAAEAISNARENIQRFFGRGAEDSLADQAANE | (SEQ. ID. NO. 141) |
| WGRSGKDPNHFRPAGLPEKY | |
| YDAAKRGPGGVWAAEAISNARENIQRFFGRGAEDSLADQAANEW | (SEQ. ID. NO. 142) |
| GRSGKDPNHFRPAGLPEKY | |
| DAAKRGPGGVWAAEAISNARENIQRFFGRGAEDSLADQAANEWG | (SEQ. ID. NO. 143) |
| RSGKDPNHFRPAGLPEKY | |
| AAKRGPGGVWAAEAISNARENIQRFFGRGAEDSLADQAANEWGR | (SEQ. ID. NO. 144) |
| SGKDPNHFRPAGLPEKY | |
| AKRGPGGVWAAEAISNARENIQRFFGRGAEDSLADQAANEWGRS | (SEQ. ID. NO. 145) |
| GKDPNHFRPAGLPEKY | |
| KRGPGGVWAAEAISNARENIQRFFGRGAEDSLADQAANEWGRSG | (SEQ. ID. NO. 146) |
| KDPNHFRPAGLPEKY | |
| RGPGGVWAAEAISNARENIQRFFGRGAEDSLADQAANEWGRSGK | (SEQ. ID. NO. 147) |
| DPNHFRPAGLPEKY | |
| GPGGVWAAEAISNARENIQRFFGRGAEDSLADQAANEWGRSGKD | (SEQ. ID. NO. 148) |
| PNHFRPAGLPEKY | |
| PGGVWAAEAISNARENIQRFFGRGAEDSLADQAANEWGRSGKDP | (SEQ. ID. NO. 149) |
| NHFRPAGLPEKY | |
| GGVWAAEAISNARENIQRFFGRGAEDSLADQAANEWGRSGKDPN | (SEQ. ID. NO. 150) |
| HFRPAGLPEKY | |
| GVWAAEAISNARENIQRFFGRGAEDSLADQAANEWGRSGKDPNH | (SEQ. ID. NO. 151) |
| FRPAGLPEKY | |
| VWAAEAISNARENIQRFFGRGAEDSLADQAANEWGRSGKDPNHF | (SEQ. ID. NO. 152) |
| RPAGLPEKY | |
| WAAEAISNARENIQRFFGRGAEDSLADQAANEWGRSGKDPNHFRP | (SEQ. ID. NO. 153) |
| AGLPEKY | |
| AAEAISNARENIQRFFGRGAEDSLADQAANEWGRSGKDPNHFRPA | (SEQ. ID. NO. 154) |
| GLPEKY | |
| AEAISNARENIQRFFGRGAEDSLADQAANEWGRSGKDPNHFRPAG | (SEQ. ID. NO. 155) |
| LPEKY | |
| EAISNARENIQRFFGRGAEDSLADQAANEWGRSGKDPNHFRPAGL | (SEQ. ID. NO. 156) |
| PEKY | |
| AISNARENIQRFFGRGAEDSLADQAANEWGRSGKDPNHFRPAGLP | (SEQ. ID. NO. 157) |
| EKY | |
| ISNARENIQRFFGRGAEDSLADQAANEWGRSGKDPNHFRPAGLPEKY | (SEQ. ID. NO. 158) |
| SNARENIQRFFGRGAEDSLADQAANEWGRSGKDPNHFRPAGLPEKY | (SEQ. ID. NO. 159) |
| NARENIQRFFGRGAEDSLADQAANEWGRSGKDPNHFRPAGLPEKY | (SEQ. ID. NO. 160) |
| ARENIQRFFGRGAEDSLADQAANEWGRSGKDPNHFRPAGLPEKY | (SEQ. ID. NO. 161) |
| RENIQRFFGRGAEDSLADQAANEWGRSGKDPNHFRPAGLPEKY | (SEQ. ID. NO. 162) |
| ENIQRFFGRGAEDSLADQAANEWGRSGKDPNHFRPAGLPEKY | (SEQ. ID. NO. 163) |
| NIQRFFGRGAEDSLADQAANEWGRSGKDPNHFRPAGLPEKY | (SEQ. ID. NO. 164) |
| IQRFFGRGAEDSLADQAANEWGRSGKDPNHFRPAGLPEKY | (SEQ. ID. NO. 165) |
| QRFFGRGAEDSLADQAANEWGRSGKDPNHFRPAGLPEKY | (SEQ. ID. NO. 166) |
| RFFGRGAEDSLADQAANEWGRSGKDPNHFRPAGLPEKY | (SEQ. ID. NO. 167) |
| FFGRGAEDSLADQAANEWGRSGKDPNHFRPAGLPEKY | (SEQ. ID. NO. 168) |
| GRGAEDSLADQAANEWGRSGKDPNHFRPAGLPEKY | (SEQ. ID. NO. 169) |
| RGAEDSLADQAANEWGRSGKDPNHFRPAGLPEKY | (SEQ. ID. NO. 170) |
| GAEDSLADQAANEWGRSGKDPNHFRPAGLPEKY | (SEQ. ID. NO. 171) |
| AEDSLADQAANEWGRSGKDPNHFRPAGLPEKY | (SEQ. ID. NO. 172) |
| EDSLADQAANEWGRSGKDPNHFRPAGLPEKY | (SEQ. ID. NO. 173) |
| DSLADQAANEWGRSGKDPNHFRPAGLPEKY | (SEQ. ID. NO. 174) |
| SLADQAANEWGRSGKDPNHFRPAGLPEKY | (SEQ. ID. NO. 175) |
| LADQAANEWGRSGKDPNHFRPAGLPEKY | (SEQ. ID. NO. 176) |
| ADQAANEWGRSGKDPNHFRPAGLPEKY | (SEQ. ID. NO. 177) |
| DQAANEWGRSGKDPNHFRPAGLPEKY | (SEQ. ID. NO. 178) |
| QAANEWGRSGKDPNHFRPAGLPEKY | (SEQ. ID. NO. 179) |
| AANEWGRSGKDPNHFRPAGLPEKY | (SEQ. ID. NO. 180) |
| ANEWGRSGKDPNHFRPAGLPEKY | (SEQ. ID. NO. 181) |
| NEWGRSGKDPNHFRPAGLPEKY | (SEQ. ID. NO. 182) |
| EWGRSGKDPNHFRPAGLPEKY | (SEQ. ID. NO. 183) |
| WGRSGKDPNHFRPAGLPEKY | (SEQ. ID. NO. 184) |
| GRSGKDPNHFRPAGLPEKY | (SEQ. ID. NO. 185) |
| RSGKDPNHFRPAGLPEKY | (SEQ. ID. NO. 186) |
| SGKDPNHFRPAGLPEKY | (SEQ. ID. NO. 187) |
| GKDPNHFRPAGLPEKY | (SEQ. ID. NO. 188) |
| KDPNHFRPAGLPEKY | (SEQ. ID. NO. 189) |
| DPNHFRPAGLPEKY | (SEQ. ID. NO. 190) |
| PNHFRPAGLPEKY | (SEQ. ID. NO. 191) |
| NHFRPAGLPEKY | (SEQ. ID. NO. 192) |
| HFRPAGLPEKY | (SEQ. ID. NO. 193) |
| FRPAGLPEKY | (SEQ. ID. NO. 194) |
| RPAGLPEKY | (SEQ. ID. NO. 195) |
| PAGLPEKY | (SEQ. ID. NO. 196) |
| AGLPEKY | (SEQ. ID. NO. 197) |
| GLPEKY | (SEQ. ID. NO. 198) |
| LPEKY | (SEQ. ID. NO. 199) |
| PEKY | (SEQ. ID. NO. 200) |
| EKY | |

Internal truncations of SAA include:
-RSF- and -RSF-5-105 wherein

5-105 means F, FS, FSF, . . .

| | |
|---|---|
| FLGEAFDGARDMWRAYSDMREANYIGSDKYFHARGNYDAAKRGPGGVWAAEAIS | (SEQ. ID. NO. 201) |
| NARENIQRFFGRGAEDSLADQAANEWGRSGKDPNHFRPAGLPEKY | |

-SFF- and -SFF-5-105 wherein

5-105 means S, SF, SFL, . . .

| | |
|---|---|
| GEAFDGARDMWRAYSDMREANYIGSDKYFHARGNYDAAKRGPG | (SEQ. ID. NO. 202) |
| GVWAAEAISNARENIQRFFGRGAEDSLADQAANEWGRSGKDPNHFRPAGLPEKY | |

-FFS- and -FFS-5-105 wherein

5-105 means F, FL, FLG, . . .

| | |
|---|---|
| FLGEAFDGARDMWRAYSDMREANYIGSDKYFHARGNYDAAKRGPG | (SEQ. ID. NO. 203) |
| GVWAAEAISNARENIQRFFGRGAEDSLADQAANEWGRSGKDPNHFRPAGLPEKY | |

-FSF- and -FSF-5-105 wherein

5-105 means L, LG, LGE, . . .

| | |
|---|---|
| FSFLGEAFDGARDMWRAYSDMREANYIGSDKYFHARGNYDAAKRGPG | (SEQ. ID. NO. 204) |
| GVWAAEAISNARENIQRFFGRGAEDSLADQAANEWGRSGKDPNHFRPAGLPEKY | |

-SFL- and -SFL-5-105 wherein

5-105 means G, GE, GEA, . . .

| | |
|---|---|
| SFLGEAFDGARDMWRAYSDMREANYIGSDKYFHARGNYDAAKR | (SEQ. ID. NO. 205) |
| GPGGVWAAEAISNARENIQRFFGRGAEDSLADQAANEWGRSGKDPNHFRPAGLPEKY | |

-FLG- and -FLG-5-105 wherein

5-105 means E, EA, EAF, . . .

| | |
|---|---|
| FLGEAFDGARDMWRAYSDMREANYIGSDKYFHARGNYDAAKRG | (SEQ. ID. NO. 206) |
| PGGVWAAEAISNARENIQRFFGRGAEDSLADQAANEWGRSGKDPNHFRPAGLPEKY | |

-LGE- and -LGE-5-105 wherein

5-105 means A, AF, AFD, . . .

| | |
|---|---|
| LGEAFDGARDMWRAYSDMREANYIGSDKYFHARGNYDAAKRGP | (SEQ. ID. NO. 207) |
| GGVWAAEAISNARENIQRFFGRGAEDSLADQAANEWGRSGKDPNHFRPAGLPEKY | |

-GEA- and -GEA-5-105 wherein

5-105 means F, FD, FDG, . . .

| | |
|---|---|
| GEAFDGARDMWRAYSDMREANYIGSDKYFHARGNYDAAKRGPG | (SEQ. ID. NO. 208) |
| GVWAAEAISNARENIQRFFGRGAEDSLADQAANEWGRSGKDPNHFRPAGLPEKY | |

-EAF- and -EAF-5-105 wherein

5-105 means D, DG, DGA, . . .

| | |
|---|---|
| EAFDGARDMWRAYSDMREANYIGSDKYFHARGNYDAAKRGPGG | (SEQ. ID. NO. 209) |
| VWAAEAISNARENIQRFFGRGAEDSLADQAANEWGRSGKDPNHFRPAGLPEKY | |

-AFD- and -AFD-5-105 wherein

5-105 means G, GA, GAR, . . .

| | |
|---|---|
| AFDGARDMWRAYSDMREANYIGSDKYFHARGNYDAAKRGPGGV | (SEQ. ID. NO. 210) |
| WAAEAISNARENIQRFFGRGAEDSLADQAANEWGRSGKDPNHFRPAGLPEKY | |

-FDG- and -FDG-5-105 wherein

5-105 means A, AR, ARD, . . .

| | |
|---|---|
| FDGARDMWRAYSDMREANYIGSDKYFHARGNYDAAKRGPGGV | (SEQ. ID. NO. 211) |
| WAAEAISNARENIQRFFGRGAEDSLADQAANEWGRSGKDPNHFRPAGLPEKY | |

-DGA- and -DGA-5-105 wherein

5-105 means R, RD, RDM, . . .

| | |
|---|---|
| DGARDMWRAYSDMREANYIGSDKYFHARGNYDAAKRGPGGVW | (SEQ. ID. NO. 212) |
| AAEAISNARENIQRFFGRGAEDSLADQAANEWGRSGKDPNHFRPAGLPEKY | |

-GAR- and -GAR-5-105 wherein

5-105 means D, DM, DMW, . . .

| | |
|---|---|
| GARDMWRAYSDMREANYIGSDKYFHARGNYDAAKRGPGGVWA | (SEQ. ID. NO. 213) |
| AEAISNARENIQRFFGRGAEDSLADQAANEWGRSGKDPNHFRPAGLPEKY | |

-ARD- and -ARD-5-105 wherein

5-105 means M, MW, M, . . .

| | |
|---|---|
| ARDMWRAYSDMREANYIGSDKYFHARGNYDAAKRGPGGVWAA | (SEQ. ID. NO. 214) |
| EAISNARENIQRFFGRGAEDSLADQAANEWGRSGKDPNHFRPAGLPEKY | |

-RDM- and -RDM-5-105 wherein

5-105 means W, WR, WRA, . . .

| | |
|---|---|
| RDMWRAYSDMREANYIGSDKYFHARGNYDAAKRGPGGVWAAE | (SEQ. ID. NO. 215) |
| AISNARENIQRFFGRGAEDSLADQAANEWGRSGKDPNHFRPAGLPEKY | |

-DMW- and -DMW-5-105 wherein

5-105 means R, RA, RAY, . . .

| | |
|---|---|
| DMWRAYSDMREANYIGSDKYFHARGNYDAAKRGPGGVWAAEAI | (SEQ. ID. NO. 216) |
| SNARENIQRFFGRGAEDSLADQAANEWGRSGKDPNHFRPAGLPEKY | |

-MWR- and -MWR-5-105 wherein

5-105 means A, AY, AYS, . . .

| | |
|---|---|
| MWRAYSDMREANYIGSDKYFHARGNYDAAKRGPGGVWAAEAIS | (SEQ. ID. NO. 217) |
| NARENIQRFFGRGAEDSLADQAANEWGRSGKDPNHFRPAGLPEKY | |

-WRA- and -WRA-5-105 wherein

5-105 means Y, YS, YSD, . . .

| | |
|---|---|
| WRAYSDMREANYIGSDKYFHARGNYDAAKRGPGGVWAAEAISN | (SEQ. ID. NO. 218) |
| ARENIQRFFGRGAEDSLADQAANEWGRSGKDPNHFRPAGLPEKY | |

-RAY- and -RAY-5-105 wherein

5-105 means S, SD, SDM, . . .

| | |
|---|---|
| RAYSDMREANYIGSDKYFHARGNYDAAKRGPGGVWAAEAISNAR | (SEQ. ID. NO. 219) |
| ENIQRFFGRGAEDSLADQAANEWGRSGKDPNHFRPAGLPEKY | |

-AYS- and -AYS-5-105 wherein

5-105 means D, DM, DMR, . . .

| | |
|---|---|
| AYSDMREANYIGSDKYFHARGNYDAAKRGPGGVWAAEAISNARE | (SEQ. ID. NO. 220) |
| NIQRFFGRGAEDSLADQAANEWGRSGKDPNHFRPAGLPEKY | |

-YSD- and -YSD-5-105 wherein

5-105 means M, MR, MRE, . . .

| | |
|---|---|
| YSDMREANYIGSDKYFHARGNYDAAKRGPGGVWAAEAISNAREN | (SEQ. ID. NO. 221) |
| IQRFFGRGAEDSLADQAANEWGRSGKDPNHFRPAGLPEKY | |

-SDM- and -SDM-5-105 wherein

5-105 means R, RE, REA, . . .

| | |
|---|---|
| SDMREANYIGSDKYFHARGNYDAAKRGPGGVWAAEAISNARENI | (SEQ. ID. NO. 222) |
| QRFFGRGAEDSLADQAANEWGRSGKDPNHFRPAGLPEKY | |

-DMR- and -DMR-5-105 wherein

5-105 means E, EA, EAN, . . .

| | |
|---|---|
| DMREANYIGSDKYFHARGNYDAAKRGPGGVWAAEAISNARENIQ | (SEQ. ID. NO. 223) |
| RFFGRGAEDSLADQAANEWGRSGKDPNHFRPAGLPEKY | |

-MRE- and -MRE-5-105 wherein

5-105 means A, AN, ANY, . . .

| | |
|---|---|
| MREANYIGSDKYFHARGNYDAAKRGPGGVWAAEAISNARENIQR | (SEQ. ID. NO. 224) |
| FFGRGAEDSLADQAANEWGRSGKDPNHFRPAGLPEKY | |

-REA- and -REA-5-105 wherein

5-105 means N, NY, NYI, . . .

| | |
|---|---|
| REANYIGSDKYFHARGNYDAAKRGPGGVWAAEAISNARENIQRFF | (SEQ. ID. NO. 225) |
| GRGAEDSLADQAANEWGRSGKDPNHFRPAGLPEKY | |

-EAN- and -EAN-5-105 wherein

5-105 means Y, YI, YIG, . . .

| | |
|---|---|
| EANYIGSDKYFHARGNYDAAKRGPGGVWAAEAISNARENIQRFFG | (SEQ. ID. NO. 226) |
| RGAEDSLADQAANEWGRSGKDPNHFRPAGLPEKY | |

-ANY- and -ANY-5-105 wherein

5-105 means I, IG, IGS, . . .

| | |
|---|---|
| ANYIGSDKYFHARGNYDAAKRGPGGVWAAEAISNARENIQRFFGR | (SEQ. ID. NO. 227) |
| GAEDSLADQAANEWGRSGKDPNHFRPAGLPEKY | |

-NYI- and -NYI-5-105 wherein

5-105 means G, GS, GSD, . . .

| | |
|---|---|
| NYIGSDKYFHARGNYDAAKRGPGGVWAAEAISNARENIQRFFGRG | (SEQ. ID. NO. 228) |
| AEDSLADQAANEWGRSGKDPNHFRPAGLPEKY | |

-YIG- and -YIG-5-105 wherein

5-105 means S, SD, SDK, . . .

| | |
|---|---|
| YIGSDKYFHARGNYDAAKRGPGGVWAAEAISNARENIQRFFGRGA | (SEQ. ID. NO. 229) |
| EDSLADQAANEWGRSGKDPNHFRPAGLPEKY | |

-IGS- and -IGS-5-105 wherein

5-105 means D, DK, DKY, . . .

| | |
|---|---|
| IGSDKYFHARGNYDAAKRGPGGVWAAEAISNARENIQRFFGRGAE | (SEQ. ID. NO. 230) |
| DSLADQAANEWGRSGKDPNHFRPAGLPEKY | |

-GSD- and -GSD-5-105 wherein

5-105 means K, KY, KYF, . . .

| | |
|---|---|
| GSDKYFHARGNYDAAKRGPGGVWAAEAISNARENIQRFFGRGAE | (SEQ. ID. NO. 231) |
| DSLADQAANEWGRSGKDPNHFRPAGLPEKY | |

-SDK- and -SDK-5-105 wherein

5-105 means Y, YF, YFH, . . .

| | |
|---|---|
| SDKYFHARGNYDAAKRGPGGVWAAEAISNARENIQRFFGRGAED | (SEQ. ID. NO. 232) |
| SLADQAANEWGRSGKDPNHFRPAGLPEKY | |

-DKY- and -DKY-5-105 wherein

5-105 means F, FH, FHA, . . .

| | |
|---|---|
| DKYFHARGNYDAAKRGPGGVWAAEAISNARENIQRFFGRGAEDS | (SEQ. ID. NO. 233) |
| LADQAANEWGRSGKDPNHFRPAGLPEKY | |

-KYF- and -KYF-5-105 wherein

5-105 means H, HA, HAR, . . .

| | |
|---|---|
| KYFHARGNYDAAKRGPGGVWAAEAISNARENIQRFFGRGAEDSL | (SEQ. ID. NO. 234) |
| ADQAANEWGRSGKDPNHFRPAGLPEKY | |

-YFH- and -YFH-5-105 wherein

5-105 means A, AR, ARG, . . .

| | |
|---|---|
| YFHARGNYDAAKRGPGGVWAAEAISNARENIQRFFGRGAEDSLA | (SEQ. ID. NO. 235) |
| DQAANEWGRSGKDPNHFRPAGLPEKY | |

-FHA- and -FHA-5-105 wherein

5-105 means R, RG, RGN, . . .

| | |
|---|---|
| FHARGNYDAAKRGPGGVWAAEAISNARENIQRFFGRGAEDSLAD | (SEQ. ID. NO. 236) |
| QAANEWGRSGKDPNHFRPAGLPEKY | |

-HAR- and -HAR-5-105 wherein

5-105 means G, GN, GNY, . . .

| | |
|---|---|
| HARGNYDAAKRGPGGVWAAEAISNARENIQRFFGRGAEDSLADQ | (SEQ. ID. NO. 237) |
| AANEWGRSGKDPNHFRPAGLPEKY | |

-ARG- and -ARG-5-105 wherein

5-105 means N, NY, NYD, . . .

| | |
|---|---|
| ARGNYDAAKRGPGGVWAAEAISNARENIQRFFGRGAEDSLADQA | (SEQ. ID. NO. 238) |
| ANEWGRSGKDPNHFRPAGLPEKY | |

-RGN- and -RGN-5-105 wherein

5-105 means Y, YD, YDA, . . .

| | |
|---|---|
| RGNYDAAKRGPGGVWAAEAISNARENIQRFFGRGAEDSLADQAA | (SEQ. ID. NO. 239) |
| NEWGRSGKDPNHFRPAGLPEKY | |

-GNY- and -GNY-5-105 wherein

5-105 means D, DA, DAA, . . .

| | |
|---|---|
| GNYDAAKRGPGGVWAAEAISNARENIQRFFGRGAEDSLADQAAN | (SEQ. ID. NO. 240) |
| EWGRSGKDPNHFRPAGLPEKY | |

-NYD- and -NYD-5-105 wherein

5-105 means A, AA, AAK, . . .

| | |
|---|---|
| NYDAAKRGPGGVWAAEAISNARENIQRFFGRGAEDSLADQAANE | (SEQ. ID. NO. 241) |
| WGRSGKDPNHFRPAGLPEKY | |

-YDA- and -YDA-5-105 wherein

5-105 means A, AK, AKR, . . .

| | |
|---|---|
| YDAAKRGPGGVWAAEAISNARENIQRFFGRGAEDSLADQAANEW | (SEQ. ID. NO. 242) |
| GRSGKDPNHFRPAGLPEKY | |

-DAA- and -DAA-5-105 wherein

5-105 means K, KR, KRG, . . .

| | |
|---|---|
| DAAKRGPGGVWAAEAISNARENIQRFFGRGAEDSLADQAANEWG | (SEQ. ID. NO. 243) |
| RSGKDPNHFRPAGLPEKY | |

-AAK- and -AAK-5-105 wherein

5-105 means R, RG, RGP, . . .

| | |
|---|---|
| AAKRGPGGVWAAEAISNARENIQRFFGRGAEDSLADQAANEWGR | (SEQ. ID. NO. 244) |
| SGKDPNHFRPAGLPEKY | |

-AKR- and -AKR-5-105 wherein

5-105 means G, GP, GPG, . . .

| | |
|---|---|
| AKRGPGGVWAAEAISNARENIQRFFGRGAEDSLADQAANEWGRS | (SEQ. ID. NO. 245) |
| GKDPNHFRPAGLPEKY | |

-KRG- and -KRG-5-105 wherein

5-105 means P, PG, PGG, . . .

| | |
|---|---|
| KRGPGGVWAAEAISNARENIQRFFGRGAEDSLADQAANEWGRSG | (SEQ. ID. NO. 245) |
| KDPNHFRPAGLPEKY | |

-RGP- and -RGP-5-105 wherein

5-105 means G, GG, GGV, . . .

| | |
|---|---|
| RGPGGVWAAEAISNARENIQRFFGRGAEDSLADQAANEWGRSGK | (SEQ. ID. NO. 247) |
| DPNHFRPAGLPEKY | |

-GPG- and -GPG-5-105 wherein

5-105 means G, GV, GVW, . . .

| | |
|---|---|
| GPGGVWAAEAISNARENIQRFFGRGAEDSLADQAANEWGRSGKD | (SEQ. ID. NO. 248) |
| PNHFRPAGLPEKY | |

-PGG- and -PGG-5-105 wherein

5-105 means V, VW, VWA, . . .

| | |
|---|---|
| PGGVWAAEAISNARENIQRFFGRGAEDSLADQAANEWGRSGKDP | (SEQ. ID. NO. 249) |
| NHFRPAGLPEKY | |

-GGV- and -GGV-5-105 wherein

5-105 means W, WA, WWA, . . .

| | |
|---|---|
| GGVWAAEAISNARENIQRFFGRGAEDSLADQAANEWGRSGKDPN | (SEQ. ID. NO. 250) |
| HFRPAGLPEKY | |

-GVW- and -GVW-5-105 wherein

5-105 means A, AA, AAE, . . .

| | |
|---|---|
| GVWAAEAISNARENIQRFFGRGAEDSLADQAANEWGRSGKDPNH | (SEQ. ID NO. 251) |
| FRPAGLPEKY | |

-VWA- and -VWA-5-105 wherein

5-105 means A, AE, AEA, . . .

| | |
|---|---|
| VWAAEAISNARENIQRFFGRGAEDSLADQAANEWGRSGKDPNHF | (SEQ. ID. NO. 252) |
| RPAGLPEKY | |

-WAA- and -WAA-5-105 wherein

5-105 means E, EA, EAI, . . .

| | |
|---|---|
| WAAEAISNARENIQRFFGRGAEDSLADQAANEWGRSGKDPNHFRP | (SEQ. ID. NO. 253) |
| AGLPEKY | |

-AAE- and -AAE-5-105 wherein

5-105 means A, AI, AIS, . . .

| | |
|---|---|
| AAEAISNARENIQRFFGRGAEDSLADQAANEWGRSGKDPNHFRPA | (SEQ. ID. NO. 254) |
| GLPEKY | |

-AEA- and -AEA-5-105 wherein

5-105 means I, IS, ISN, . . .

| | |
|---|---|
| AEAISNARENIQRFFGRGAEDSLADQAANEWGRSGKDPNHFRPAG | (SEQ. ID. NO. 255) |
| LPEKY | |

-EAI- and -EAI-5-105 wherein

5-105 means S, SN, SNA, . . .

| | |
|---|---|
| EAISNARENIQRFFGRGAEDSLADQAANEWGR | (SEQ. ID. NO. 256) |
| SGKDPNHFRPAGLPEKY | |

-AIS- and -AIS-5-105 wherein

5-105 means N, NA, NAR, . . .

| | |
|---|---|
| AISNARENIQRFFGRGAEDSLADQAANEWGRS | (SEQ. ID. NO. 257) |
| GKDPNHFRPAGLPEKY | |

-ISN- and -ISN-5-105 wherein

5-105 means A, AR, ARE, . . .

| | |
|---|---|
| ISNARENIQRFFGRGAEDSLADQAANEWGRSG | (SEQ. ID. NO. 258) |
| KDPNHFRPAGLPEKY | |

-SNA- and -SNA-5-105 wherein

5-105 means R, RE, REN, . . .

| | |
|---|---|
| SNARENIQRFFGRGAEDSLADQAANEWGRSGK | (SEQ. ID. NO. 259) |
| DPNHFRPAGLPEKY | |

-NAR- and -NAR-5-105 wherein

5-105 means E, EN, ENI, . . .

| | |
|---|---|
| NARENIQRFFGRGAEDSLADQAANEWGRSGKD | (SEQ. ID. NO. 260) |
| PNHFRPAGLPEKY | |

-ARE- and -ARE-5-105 wherein

5-105 means N, NI, NIQ, . . .

| | |
|---|---|
| ARENIQRFFGRGAEDSLADQAANEWGRSGKDP | (SEQ. ID. NO. 261) |
| NHFRPAGLPEKY | |

-REN- and -REN-5-105 wherein

5-105 means I, IQ, IQR, . . .

| | |
|---|---|
| RENIQRFFGRGAEDSLADQAANEWGRSGKDPN | (SEQ. ID. NO. 262) |
| HFRPAGLPEKY | |

-ENI- and -ENI-5-105 wherein

5-105 means Q, QR, QRF, . . .

| | |
|---|---|
| ENIQRFFGRGAEDSLADQAANEWGRSGKDPNH | (SEQ. ID. NO. 263) |
| FRPAGLPEKY | |

-NIQ- and -NIQ-5-105 wherein

5-105 means R, RF, RFF, . . .

| | |
|---|---|
| NIQRFFGRGAEDSLADQAANEWGRSGKDPNHF | (SEQ. ID. NO. 264) |
| RPAGLPEKY | |

-IQR- and -IQR-5-105 wherein

5-105 means F, FF, FFG, . . .

| | |
|---|---|
| IQRFFGRGAEDSLADQAANEWGRSGKDPNHFR | (SEQ. ID. NO. 265) |
| PAGLPEKY | |

-QRF- and -QRF-5-105 wherein

5-105 means F, FG, FGR, . . .

(SEQ. ID. NO. 266)

QRFFGRGAEDSLADQAANEWGRSGKDPNHFRPAGLPEKY

-RFF- and -RFF-5-105 wherein

5-105 means G, GR, GRG, . . .

(SEQ. ID. NO. 267)

RFFGRGAEDSLADQAANEWGRSGKDPNHFRPAGLPEKY

-FFG- and -FFG-5-105 wherein

5-105 means R, RG, RGA, . . .

(SEQ. ID. NO. 268)

FFGRGAEDSLADQAANEWGRSGKDPNHFRPAGLPEKY

-FGR- and -FGR-5-105 wherein

5-105 means G, GA, GAE, . . .

(SEQ. ID. NO. 269)

FGRGAEDSLADQAANEWGRSGKDPNHFRPAGLPEKY

-GRG- and -GRG-5-105 wherein

5-105 means A, AE, AED, . . .

(SEQ. ID. NO. 270)

GRGAEDSLADQAANEWGRSGKDPNHFRPAGLPEKY

-RGA- and -RGA-5-105 wherein

5-105 means E, ED, EDS, . . .

(SEQ. ID. NO. 271)

RGAEDSLADQAANEWGRSGKDPNHFRPAGLPEKY

-GAE- and -GAE-5-105 wherein

5-105 means D, DS, DSL, . . .

(SEQ. ID. NO. 272)

GAEDSLADQAANEWGRSGKDPNHFRPAGLPEKY

-AED- and -AED-5-105 wherein

5-105 means S, SL, SLA, . . .

AEDSLADQAANEWGRSGKDPNHFRPAGLPEKY (SEQ. ID. NO. 273)
-EDS- and -EDS-5-105 wherein

5-105 means L, LA, LAD, . . .

EDSLADQAANEWGRSGKDPNHFRPAGLPEKY (SEQ. ID. NO. 274)
-DSL- and -DSL-5-105 wherein

5-105 means A, AD, ADQ, . . .

DSLADQAANEWGRSGKDPNHFRPAGLPEKY (SEQ. ID. NO. 275)
-SLA- and -SLA-5-105 wherein

5-105 means D, DQ, DQA, . . .

SLADQAANEWGRSGKDPNHFRPAGLPEKY (SEQ. ID. NO. 276)
-LAD- and -LAD-5-105 wherein

5-105 means Q, QA, QAA, . . .

LADQAANEWGRSGKDPNHFRPAGLPEKY (SEQ. ID. NO. 277)
-ADQ- and -ADQ-5-105 wherein

5-105 means A, AA, AAN, . . .

ADQAANEWGRSGKDPNHFRPAGLPEKY (SEQ. ID. NO. 278)
-DQA- and -DQA-5-105 wherein

5-105 means A, AN, ANE, . . .

DQAANEWGRSGKDPNHFRPAGLPEKY (SEQ. ID. NO. 279)
-QAA- and -QAA-5-105 wherein

5-105 means N, NE, NEW, . . .

QAANEWGRSGKDPNHFRPAGLPEKY (SEQ. ID. NO. 280)
-AAN- and -AAN-5-105 wherein

5-105 means E, EW, EWG, . . .

AANEWGRSGKDPNHFRPAGLPEKY (SEQ. ID. NO. 281)
-ANE- and -ANE-5-105 wherein

5-105 means W, WG, WGR, . . .

ANEWGRSGKDPNHFRPAGLPEKY (SEQ. ID. NO. 282)
-NEW- and -NEW-5-105 wherein

5-105 means G, GR, GRS, . . .

NEWGRSGKDPNHFRPAGLPEKY (SEQ. ID. NO. 283)
-EWG- and -EWG-5-105 wherein

5-105 means R, RS, RSG, . . .

EWGRSGKDPNHFRPAGLPEKY (SEQ. ID. NO. 284)
-WGR- and -WGR-5-105 wherein

5-105 means S, SG, SGK, . . .

WGRSGKDPNHFRPAGLPEKY (SEQ. ID. NO. 285)
-GRS- and -GRS-5-105 wherein

5-105 means G, GK, GKD, . . .

GRSGKDPNHFRPAGLPEKY (SEQ. ID. NO.286)
-RSG- and -RSG-5-105 wherein

5-105 means K, KD, KDP, . . .

RSGKDPNHFRPAGLPEKY (SEQ. ID. NO. 287)
-SGK- and -SGK-5-105 wherein

5-105 means D, DP, DPN, . . .

SGKDPNHFRPAGLPEKY (SEQ. ID. NO. 288)
-GKD- and -GKD-5-105 wherein

5-105 means P, PN, PNH, . . .

GKDPNHFRPAGLPEKY (SEQ. ID. NO. 289)
-KDP- and -KDP-5-105 wherein

5-105 means N, NH, NHF, . . .

KDPNHFRPAGLPEKY (SEQ. ID. NO. 290)
-DPN- and -DPN-5-105 wherein

5-105 means H, HF, HFR, . . .

DPNHFRPAGLPEKY (SEQ. ID. NO. 291)
-PNH- and -PNH-5-105 wherein

5-105 means F, FR, FRP, . . .

PNHFRPAGLPEKY (SEQ. ID. NO. 292)
-NHF- and -NHF-5-105 wherein

5-105 means R, RP, RPA, . . .

NHFRPAGLPEKY (SEQ. ID. NO. 293)
-HFR- and -HFR-5-105 wherein

5-105 means P, PA, PAG, . . .

HFRPAGLPEKY (SEQ. ID. NO. 294)
-FRP- and -FRP-5-105 wherein
5-105 means A, AG, AGL, . . .

FRPAGLPEKY (SEQ. ID. NO. 295)
-RPA- and -RPA-5-105 wherein

5-105 means G, GL, GLP, . . .

RPAGLPEKY (SEQ. ID. NO. 296) -PAG- and -PAG-5-105 wherein

5-105 means L, LP, LPE, . . .

PAGLPEKY (SEQ. ID. NO. 297)
-AGL- and -AGL-5-105 wherein

5-105 means P, PE, PEK, . . .

AGLPEKY (SEQ. ID. NO. 298)
-GLP- and -GLP-5-105 wherein

5-105 means E, EK, EKY, . . .

GLPEKY (SEQ. ID. NO. 299)
-LPE- and -LEP-5-105 wherein

5-105 means K, KY

LPEKY (SEQ. ID. NO. 300)
-PEK- and -PEK-5-105 wherein

5-105 means Y, . . .

PEKY (SEQ. ID. NO. 301)
-EKY-

The peptides described above are preferably analyzed in SAA/FPRL1 characterization assays to determine their effect on the assembly of the SAA/FPRL1 complex and/or SAA/FPRL1-mediated signal transduction. One of skill in the art will appreciate that carboxy truncations, amino truncations, and internal truncations of the FPRL1 protein can be made in a similar fashion and these molecules can be characterized in SAA/FPRL1 characterization assays.

In many embodiments, we use isolated or purified SAA, FPRL1, or fragments thereof. The term “isolated” requires that the material be removed from its original environment (e.g., the natural environment if it is naturally occurring). For example, a naturally occurring protein present in a living cell is not isolated, but the same protein, separated from some or all of the coexisting materials in the natural system, is isolated. The term “purified” does not require absolute purity; rather it is intended as a relative definition. For example, proteins are routinely purified to electrophoretic homogeneity, as detected by Coomassie staining, and are suitable in several assays despite having the presence of contaminants.

In the dislcosure below, we teach the preparation of multimeric supports having peptide agents that interact with FPRL1 or SAA and thereby modulate a cellular response. These multimeric supports have many uses including, but not limited to, the manufacture of biotechnological tools and components for pharmaceuticals, therapeutic and prophylactic agents.

Preparation of Multimeric Supports and Multimerized Ligands

In order to be useful as a biotechnological tool or a component to a prophylactic or therapeutic agent, it is desirable to provide a peptide agent in such a form or in such a way that a sufficient affinity for FPRL1 or SAA is obtained. While a natural monomeric peptide agent (e.g., SAA and fragments thereof appearing as discrete units of the peptide each carrying only one binding epitope) is sufficient to interact with FPRL1 and thereby modulate a cellular response, synthetic ligands or multimeric ligands (e.g., SAA and fragments thereof appearing as multiple units of the peptide agent with several binding epitopes) can have far greater ability to interact with FPRL1 and thereby modulate a cellular response. It should be noted that the term “multimeric” is meant to refer to the presence of more than one unit of a ligand, for example several individual molecules of SAA or fragments thereof, as distinguished from the term “multimerized” which refers to the presence of more than one ligand joined as a single discrete unit, for example several molecules of SAA or fragments thereof joined in tandem.

A multimeric agent (synthetic or natural) that modulates a cellular response by effecting SAA/FPRL1 assembly or SAA/FPRL1-mediated signal transduction can be obtained by joining SAA or FPRL1, a fragment of SAA or FPRL1, or a peptidomimetic that resembles these molecules to a macromolecular support. A “support” can also be termed a carrier, a resin or any macromolecular structure used to attach or immobilize a peptide agent. Solid supports include, but are not limited to, the walls of wells of a reaction tray, test tubes, polystyrene beads, magnetic beads, nitrocellulose strips, membranes, microparticles such as latex particles, sheep (or other animal) red blood cells, artificial cells and others. The macromolecular support can have a hydrophobic surface that interacts with a portion of the peptide agent by hydrophobic non-covalent interaction. The hydrophobic surface of the support can also be a polymer such as plastic or any other polymer in which hydrophobic groups have been linked such as polystyrene, polyethylene or polyvinyl. Alternatively, the peptide agent can be covalently bound to carriers including proteins and oligo/polysaccarides (e.g. cellulose, starch, glycogen, chitosane or aminated sepharose). In these later embodiments, a reactive group on the peptide agent, such as a hydroxy or an amino group, can be used to join to a reactive group on the carrier so as to create the covalent bond. The support can also have a charged surface that interacts with the peptide agent. Additionally, the support can have other reactive groups which can be chemically activated so as to attach a peptide agent. For example, cyanogen bromide activated matrices, epoxy activated matrices, thio and thiopropyl gels, nitrophenyl chloroformate and N-hydroxy succinimide chlorformate linkages, and oxirane acrylic supports are common in the art. (Sigma).

The support can also comprise an inorganic carrier such as silicon oxide material (e.g. silica gel, zeolite, diatomaceous earth or aminated glass) to which the peptide agent is covalently linked through a hydroxy, carboxy or amino group and a reactive group on the carrier. Furthermore, in some embodiments, a liposome or lipid bilayer (natural or synthetic) is contemplated as a support and peptide agents are attached to the membrane surface or are incorporated into the membrane by techniques in liposome engineering. By one approach, liposome multimeric supports comprise a peptide agent that is exposed on the surface of the bilayer and a second domain which anchors the peptide agent to the lipid bilayer. The anchor can be constructed of hydrophobic amino acid residues, resembling known transmembrane domains, or can comprise ceramides that are attached to the first domain by conventional techniques.

Carriers for use in the body, (i.e. for prophylactic or therapeutic applications) are desirably physiological, non-toxic and preferably, non-immunoresponsive. Suitable carriers for use in the body include poly-L-lysine, poly-D, L-alanine, liposomes, and Chromosorb® (Johns-Manville Products, Denver Colo.). Ligand conjugated Chromosorb® (Synsorb-Pk) has been tested in humans for the prevention of hemolytic-uremic syndrome and was reported as not presenting adverse reactions. (Armstrong et al. J. Infectious Diseases 171:1042-1045 (1995)). For some embodiments, a “naked” carrier (i.e., lacking an attached peptide agent) that has the capacity to attach a peptide agent in the body of a subject is administered. By this approach, a “prodrug-type” therapy is envisioned in which the naked carrier is administered separately from the peptide agent and, once both are in the body of the subject, the carrier and the peptide agent are assembled into a multimeric complex.

The insertion of linkers, such as 8 linkers, of an appropriate length between the peptide agent and the support are also contemplated so as to encourage greater flexibility of the peptide agent and thereby overcome any steric hindrance that can be presented by the support. The determination of an appropriate length of linker that allows for an optimal cellular response or lack thereof, can be determined by screening the peptide agents with varying linkers in the assays detailed in the present disclosure.

A composite support comprising more than one type of peptide agent is also envisioned. A “composite support” can be a carrier, a resin, or any macromolecular structure used to attach or immobilize two or more different peptide agents that modulate a cellular response by effecting SAA/FPRL1 assembly or SAA/FPRL1-mediated signal transduction. In some embodiments, a liposome or lipid bilayer (natural or synthetic) is contemplated for use in constructing a composite support and peptide agents are attached to the membrane surface or are incorporated into the membrane using techniques in liposome engineering.

As above, the insertion of linkers, such as 8 linkers, of an appropriate length between the peptide agent and the support is also contemplated so as to encourage greater flexibility in the molecule and thereby overcome any steric hindrance that can occur. The determination of an appropriate length of linker that allows for an optimal cellular response or lack thereof, can be determined by screening the ligands with varying linkers in the assays detailed in the present disclosure.

In other embodiments of the present invention, the multimeric and composite supports discussed above can have attached multimerized ligands so as to create a “multimerized-multimeric support” and a “multimerized-composite support”, respectively. A multimerized ligand can, for example, be obtained by coupling two or more peptide agents in tandem using conventional techniques in molecular biology. The multimerized form of the ligand can be advantageous for many applications because of the ability to obtain an agent with a better ability modulate assembly of the SAA/FPRL1 complex and/or SAA/FPRL1-mediated signal transduction and, thereby, effect a cellular response. We further contemplate that the incorporation of linkers or spacers, such as flexible 8 linkers, between the individual domains that make-up the multimerized agent can be advantageous. The insertion of 8 linkers of an appropriate length between protein binding domains, for example, can encourage greater flexibility in the molecule and can overcome steric hindrance. Similarly, the insertion of linkers between the multimerized ligand and the support can encourage greater flexibility and limit steric hindrance presented by the support. The determination of an appropriate length of linker that allows for an optimal effect on SAA/FPRL1 assembly or SAA/FPRL1-mediated signal transduction, can be determined by screening the ligands with varying linkers in the assays detailed in this disclosure.

In preferable embodiments, the various types of supports discussed above are created using SAA, FPRL1, a fragment of SAA or FPRL1, or a peptidomimetic that resembles these molecules. The multimeric supports, composite supports, multimerized-multimeric supports, or multimerized-composite supports, collectively referred to as “support-bound agents”, are also preferably constructed using SAA, FPRL1, a fragment of SAA or FPRL1, or a peptidomimetic that resembles these molecules.

In the following section, we provide several assay methods, which are embodiments of the invention.

Methods to Identify SAA/FPRL1 Assembly and Agents That Modulate SAA/FPRL1 Complex Assembly

We have discovered that SAA efficiently associates with FPRL1 to form a SAA/FPRL1 complex. The association of SAA to FPRL1 can be measured using many techniques common to molecular biology. By one approach, assembly of the SAA/FPRL1 complex is analyzed by contacting a support having FPRL1 or a representative fragment thereof with SAA or a representative fragment of SAA. If the SAA or fragment thereof is detectably labeled (e.g., ¹²⁵I), the association to immobilized FPRL1 (or FPRL1 fragment) can be directly determined by detecting the signal (e.g., scintillation counting). Alternatively, the association of SAA or fragment thereof with FPRL1 can be determined indirectly by employing a detectably labeled antibody that has an epitope that corresponds to a region of SAA. In these assays, the support can be a resin, plastic, a membrane, a lipid, and a cell. Additionally, the SAA can be joined to a second support. Many SAA/FPRL1 characterization assays can be automated (e.g., high throughput screening employing a fluorescently labeled SAA or fragment of SAA) so as to quickly identify regions of the molecule that are involved in binding to FPRL1. Values or results from these assays can be recorded on a computer readable media (e.g., in a database) and analyzed with a search program and retrieval program. Of course, embodiments of the invention include the converse of the assay described above. That is, immobilizing SAA or fragments thereof on a support and detecting the adhesion of labeled FPRL1 or fragments of FPRL1.

Additional embodiments include methods of identifying agents that modulate assembly of the SAA/FPRL1 complex. By one approach, an agent that modulates assembly of the SAA/FPRL1 complex can be identified by contacting a support having FPRL1 or a representative fragment thereof with SAA or a fragment of SAA in the presence of the agent. Detection of SAA dependent adhesion is accomplished, as described above, and successful agents are identified according to their ability to induce a desired modulation of the formation of the SAA-FPRL1 complex. As above, the support can be a resin, a membrane, plastic, a lipid, or a cell and the SAA can be joined to a second support. In another approach, a support having SAA or a representative fragment thereof can be used to capture directly or indirectly labeled FPRL1 or fragments of FPRL1. In some aspects, the fragments of SAA that are used have a polypeptide sequence that binds to FPRL1 and is at least 80% homologous to SAA. As above, binding is conducted in the presence of the agent and SAA dependent adhesion to FPRL1 is determined by the amount of labeled FPRL1 bound to the immobilized SAA. In this method, the support can be a resin, a membrane, plastic, a lipid, and a cell and the FPRL1 can also be joined to a second support to approximate native binding conditions.

In a preferred approach, an agent that modulates assembly of the SAA/FPRL1 complex is identified using a cell-based assay. Accordingly, cells are transfected with a construct comprising a nucleic acid sequence encoding FPRL1 or a representative fragment thereof that is involved in the assembly of the SAA/FPRL1 complex. Transfectants are brought in contact with labeled SAA or a fragment thereof and, as above, binding is conducted in the presence of the agent. SAA dependent adhesion to FPRL1 is determined by the amount of labeled SAA bound to the FPRL1 expressing cells. Additionally, a two-support adhesion assay can be employed in which a first cell that expresses FPRL1 and a second support having SAA are contacted in the presence of a peptide agent. Accordingly, the inhibition of aggregation of the two supports in the presence of the peptide agent indicates that the agent is effective at disrupting assembly of the SAA/FPRL1 complex.

In some aspects of the invention, nucleic acids encoding SAA, nucleic acids complementary to SAA, SAA protein, and polypeptide fragments of SAA are agents that modulate (e.g., inhibit or enhance) the formation of the SAA-FPRL1 complex. Several embodiments are provided that inhibit the association of SAA in a SAA/FPRL1 complex, for example, and thus, effect a cellular response. One embodiment of an SAA/FPRL1 inhibitory agent is an antisense oligonucleotide or ribozyme that hybridizes to nucleic acid encoding regions of SAA or FPRL1. By “antisense oligonucleotide” is meant a nucleic acid or modified nucleic acid including, but not limited to DNA, RNA, modified DNA or RNA (including branched chain nucleic acids and 2′ O-methyl RNA) and PNA (polyamide nucleic acid).

Several ribozymes known to those of skill in the art can be easily designed to hybridize to nucleic acid sequence encoding SAA or FPRL1 and thereby inhibit the production of functional protein. Desirably, antisense oligonucleotides or ribozymes that hybridize to the start codon of SAA or FPRL1 are used. In one embodiment, full length antisense SAA is used to significantly reduced assembly of the SAA/FPRL1 complex. Many other antisense oligonucleotides or ribozymes that interfere with the assembly of the SAA/FPRL1 complex can be designed and screened by the methods detailed previously.

The antisense nucleic acids should have a length and melting temperature sufficient to permit formation of an intracellular duplex having sufficient stability to inhibit the expression of the mRNA in the duplex. Strategies for designing antisense nucleic acids suitable for use in gene therapy are disclosed in Green et al., Ann. Rev. Biochem., 55:569-597 (1986) and Izant and Weintraub, Cell, 36:1007-1015 (1984). In some strategies, antisense molecules are obtained from a nucleotide sequence encoding SAA or FPRL1 by reversing the orientation of the coding region with respect to a promoter so as to transcribe the opposite strand from that which is normally transcribed in the cell. Antisense molecules and ribozymes can be prepared by any method known in the art for the synthesis of RNA molecules. These include techniques for chemically synthesizing oligonucleotides such as solid phase phosphoramidite chemical synthesis.

Additionally, RNA molecules can be generated by in vitro and in vivo transcription of DNA sequences encoding SAA or FPRL1. Such DNA sequences can be incorporated into a wide variety of vectors with suitable RNA polymerase promoters such as T7 or SP6. Further, antisense cDNA constructs that synthesize antisense RNA constitutively or inducibly can be introduced into cell lines, cells or tissues. Still further, oligonucleotides that are complementary to the mRNA encoding SAA or FPRL1 can be synthesized in vitro. Thus, antisense nucleic acids are capable of hybridizing to SAA or FPRL1 mRNA to create a duplex. In some embodiments, the antisense sequences can contain modified sugar phosphate backbones to increase stability and make them less sensitive to RNase activity. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine and wybutosine as well as acetyl-, methyl-, thio- and similarly modified forms of adenine, cytidine, guanine, thymine, and uridine that are not as easily recognized by endogenous endonucleases. Further examples are described by Rossi et al., Pharmacol. Ther., 50(2):245-254, (1991).

Various types of antisense oligonucleotides complementary to the SAA or FPRL1 mRNA can be used. In one preferred embodiment, stable and semi-stable antisense oligonucleotides described in International Application No. PCT WO94/23026, hereby incorporated by reference, are used. In these molecules, the 3 prime end or both the 3 and 5 prime ends are engaged in intramolecular hydrogen bonding between complementary base pairs. These molecules are better able to withstand exonuclease attacks and exhibit increased stability compared to conventional antisense oligonucleotides. In another preferred embodiment, the antisense oligodeoxynucleotides described in International Application No. WO 95/04141 are used. In yet another preferred embodiment, the covalently cross-linked antisense oligonucleotides described in International Application No. WO 96/31523 are used. These double- or single-stranded oligonucleotides comprise one or more, respectively, inter- or intra-oligonucleotide covalent cross-linkages, wherein the linkage consists of an amide bond between a primary amine group of one strand and a carboxyl group of the other strand or of the same strand, respectively, the primary amine group being directly substituted in the 2′ position of the strand nucleotide monosaccharide ring, and the carboxyl group being carried by an aliphatic spacer group substituted on a nucleotide or nucleotide analog of the other strand or the same strand, respectively.

The antisense oligodeoxynucleotides and oligonucleotides disclosed in International Application No. WO 92/18522 can also be used. These molecules are stable to degradation and contain at least one transcription control recognition sequence that binds to control proteins and are effective as decoys therefor. These molecules can contain “hairpin” structures, “dumbbell” structures, “modified dumbbell” structures, “cross-linked” decoy structures and “loop” structures. In another preferred embodiment, the cyclic double-stranded oligonucleotides described in European Patent Application No. 0 572 287 A2 are used. These ligated oligonucleotide “dumbbells” contain the binding site for a transcription factor and inhibit expression of the gene under control of the transcription factor by sequestering the factor. Use of the closed antisense oligonucleotides disclosed in International Application No. WO 92/19732, hereby incorporated by reference, is also contemplated. Because these molecules have no free ends, they are more resistant to degradation by exonucleases than are conventional oligonucleotides. These oligonucleotides can be multifunctional, interacting with several regions that are not adjacent to the target mRNA.

The appropriate level of antisense nucleic acids required to inhibit assembly of the SAA/FPRL1 complex can be determined using in vitro expression analysis and the SAA/FPRL1 characterization assays described herein. The antisense molecule can be introduced into the cells expressing SAA or FPRL1 by diffusion, injection, infection or transfection using procedures known in the art. For example, the antisense nucleic acids can be introduced into the body as a bare or naked oligonucleotide, oligonucleotide encapsulated in lipid, oligonucleotide sequence encapsidated by viral protein, or as an oligonucleotide operably linked to a promoter contained in an expression vector. The expression vector can be any of a variety of expression vectors known in the art, including retroviral or viral vectors, vectors capable of extrachromosomal replication, or integrating vectors. The vectors can be DNA or RNA.

The antisense molecules are introduced onto cell samples at a number of different concentrations preferably between 1×10⁻¹⁰M to 1×10⁻⁴M. Once the minimum concentration that can adequately control gene expression is identified, the optimized dose is translated into a dosage suitable for use in vivo. For example, an inhibiting concentration in culture of 1×10⁻⁷ translates into a dose of approximately 0.6 mg/kg bodyweight. Levels of oligonucleotide approaching 100 mg/kg bodyweight or higher can be possible after testing the toxicity of the oligonucleotide in laboratory animals. It is additionally contemplated that cells from a vertebrate, such as a mammal or human, are removed, treated with the antisense oligonucleotide, and reintroduced into the vertebrate.

Ribozymes can also be used to reduce or eliminate SAA or FPRL1 expression. Ribozymes are enzymatic RNA molecules capable of catalyzing the specific cleavage of RNA. The mechanism of ribozyme action involves sequence-specific hybridization of the ribozyme molecule to complementary target RNA, followed by endonucleolytic cleavage. Within the scope of aspects of the invention, are engineered hammerhead motif ribozyme molecules that can specifically and efficiently catalyze endonucleolytic cleavage of a sequence encoding SAA or FPRL1, for example. Specific ribozyme cleavage sites within any potential RNA target are initially identified by scanning the target molecule for ribozyme cleavage sites that include the following sequences, GUA, GUU and GUC. Once identified, short RNA sequences of between 15 and 20 ribonucleotides corresponding to the region of the target gene containing the cleavage site can be evaluated for secondary structural features that can render the oligonucleotide inoperable. The suitability of candidate targets can also be evaluated by testing accessibility to hybridization with complementary oligonucleotides using ribonuclease protection assays. Delivery of antisense and ribozyme agents by transfection and by liposome are quite well known in the art.

Another embodiment of an SAA/FPRL1 inhibitory agent is a polypeptide that interferes with the assembly of the SAA/FPRL1 complex. Polypeptide fragments that inhibit the assembly of the SAA/FPRL1 complex can be rapidly engineered and identified given the present disclosure and candidate polypeptides can contain regions of FPRL1 or SAA. The screening of polypeptide fragments and mutant proteins that inhibit assembly of the SAA/FPRL1 complex would be routine given the present disclosure and assays described herein. For example, polypeptide inhibitory agents can be identified by their ability to disrupt assembly of the SAA/FPRL1 complex by employing conventional affinity chromatography techniques, sandwich assays, ELISA assays, or other binding assays known to those of skill in the art and described above.

In another embodiment, concentrations of FPRL1 or SAA are raised in a cell so as to enhance assembly of the SAA/FPRL1 complex. There can be many ways to raise the concentration of FPRL1 or SAA in a cell. Liposome-mediated transfer, is one approach to deliver FPRL1 or SAA to a cell. Alternatively, the concentration of FPRL1 or SAA can be raised in a cell by transfecting constructs encoding these molecules. A construct for use in the transfection of FPRL1 into cells in culture is described and many others can be developed by those of skill in the art. Retroviral constructs for the delivery of nucleic acid encoding FPRL1 or SAA or fragments thereof or complements thereof are also contemplated and many retroviral vectors can be engineered accordingly. Other embodiments of inhibitory or enhancing agents (collectively refered to as “modulators”) include antibodies, peptidomimetics, and chemicals that inhibit or enhance assembly of the SAA/FPRL1 complex. Several other methods for identifying agents that modulate are discussed below.

In the discussion that follows, we describe several methods of molecular modeling and rational drug design for the identification of ligands that resemble SAA and interact with FPRL1 or vice versa to effect cellular responses including, but not limited to, signal transduction, chemotaxis, leukocyte migration, immune system response, amyloidosis, inflammatory response, infection, organ rejection, arthritis, atherosclerosis, and neoplasia.

Methods of Rational Drug Design

Rational drug design involving polypeptides requires identifying and defining a first peptide with which the designed drug is to interact, and using the first target peptide to define the requirements for a second peptide. With such requirements defined, one can find or prepare an appropriate peptide or non-peptide ligand that meets all or substantially all of the defined requirements. Thus, one goal of rational drug design is to produce structural or functional analogs of biologically active polypeptides of interest or of small molecules with which they interact (e.g., agonists, antagonists, null compounds) in order to fashion drugs that are, for example, more or less potent forms of the ligand. (See, e.g., Hodgson, Bio. Technology 9:19-21 (1991)). An example of rational drug design is the development of HIV protease inhibitors. (Erickson et al., Science 249:527-533 (1990)). Combinatorial chemistry is the science of synthesizing and testing compounds for bioactivity en masse, instead of one by one, the aim being to discover drugs and materials more quickly and inexpensively than was formerly possible. Rational drug design and combinatorial chemistry have become more intimately related in recent years due to the development of approaches in computer-aided protein modeling and drug discovery. See e.g., U.S. Pat. Nos. 4,908,773; 5,884,230; 5,873,052; 5,331,573; and 5,888,738.

The use of molecular modeling as a combinatorial chemistry tool has dramatically increased due to the advent of computer graphics. Not only is it possible to view molecules on computer screens in three dimensions but it is also possible to examine the interactions of ligands with various macromolecules such as enzymes and receptors and rationally design derivative molecules to test. (See Boorman, Chem. Eng. News 70:18-26 (1992). A vast amount of user-friendly software and hardware is now available and virtually all pharmaceutical companies have computer modeling groups devoted to rational drug design. Molecular Simulations Inc., for example, (www.msi.com) sells several sophisticated programs that allow a user to start from an amino acid sequence, build a two or three-dimensional model of the protein or polypeptide, compare it to other two and three-dimensional models, and analyze the interactions of compounds, drugs, and peptides with a three dimensional model in real time.

Accordingly, in some embodiments of the invention, software is used to compare regions of SAA or FPRL1 and fragments thereof that are involved in the assembly of the SAA/FPRL1 complex with other molecules, such as peptides, peptidomimetics, and chemicals, so that therapeutic interactions of new molecules (e.g., ligands for FPRL1) can be predicted and new derivative molecules can be designed. (Schneider, Genetic Engineering News December: page 20 (1998), Tempczyk et al., Molecular Simulations Inc. Solutions April (1997) and Butenhof, Molecular Simulations Inc. Case Notes (August 1998)). Libraries of molecules that resemble SAA or FPRL1 or that effect assembly of the SAA/FPRL1 complex and, thereby inhibit or enhance the function of FPRL1 (e.g., “modulate” FPRL1 induced cellular response) can be created. In some contexts, the term “FPRL1 modulating agent” or “modulators” includes SAA or FPRL1, polypeptide fragments corresponding to SAA or FPRL1, fusion proteins comprising SAA or FPRL1 or polypeptide fragments of these molecules, nucleic acids encoding these molecules, as well as, peptidomimetics, chemicals, and other molecules that modulate assembly of the SAA/FPRL1 complex.

As a starting point to rational drug design, a two or three dimensional model of a polypeptide of interest is created (e.g., SAA, FPRL1, and/or fragments thereof). In the past, the three-dimensional structure of proteins has been determined in a number of ways. Perhaps the best known way of determining protein structure involves the use of x-ray crystallography. A general review of this technique can be found in Van Holde, K. E. Physical Biochemistry, Prentice-Hall, N.J. pp. 221-239 (1971). Using this technique, it is possible to elucidate three-dimensional structure with good precision. Additionally, protein structure can be determined through the use of techniques of neutron diffraction, or by nuclear magnetic resonance (NMR). (See, e.g., Moore, W. J., Physical Chemistry, 4^{th} Edition, Prentice-Hall, N.J. (1972)).

Alternatively, the protein model embodiments of the present invention can be constructed using computer-based protein modeling techniques. By one approach, the protein folding problem is solved by finding target sequences that are most compatible with profiles representing the structural environments of the residues in known three-dimensional protein structures. (See, e.g., U.S. Pat. No. 5,436,850). In another technique, the known three-dimensional structures of proteins in a given family are superimposed to define the structurally conserved regions in that family. This protein modeling technique also uses the known three-dimensional structure of a homologous protein to approximate the structure of a polypeptide of interest. (See e.g., U.S. Pat. Nos. 5,557,535; 5,884,230; and 5,873,052). Conventional homology modeling techniques have been used routinely to build models of proteases and antibodies. (Sowdhamini et al., Protein Engineering 10:207, 215 (1997)). Comparative approaches can also be used to develop three-dimensional protein models when the protein of interest has poor sequence identity to template proteins. In some cases, proteins fold into similar three-dimensional structures despite having very weak sequence identities. For example, the three-dimensional structures of a number of helical cytokines fold in similar three-dimensional topology in spite of weak sequence homology.

The recent development of threading methods and “fuzzy” approaches now enables the identification of likely folding patterns and functional protein domains in a number of situations where the structural relatedness between target and template(s) is not detectable at the sequence level. By one method, fold recognition is performed using Multiple Sequence Threading (MST) and structural equivalences are deduced from the threading output using the distance geometry program DRAGON that constructs a low resolution model. A full-atom representation is then constructed using a molecular modeling package such as QUANTA.

According to this 3-step approach, candidate templates are first identified by using the novel fold recognition algorithm MST, which is capable of performing simultaneous threading of multiple aligned sequences onto one or more 3-D structures. In a second step, the structural equivalences obtained from the MST output are converted into interresidue distance restraints and fed into the distance geometry program DRAGON, together with auxiliary information obtained from secondary structure predictions. The program combines the restraints in an unbiased manner and rapidly generates a large number of low resolution model confirmations. In a third step, these low resolution model confirmations are converted into full-atom models and subjected to energy minimization using the molecular modeling package QUANTA. (See e.g., Aszódi et al., Proteins:Structure, Function, and Genetics, Supplement 1:38-42 (1997)).

In a preferred approach, the commercially available “Insight II 98” program (Molecular Simulations Inc.) and accompanying modules are used to create a two and/or three dimensional model of a polypeptide of interest (e.g., SAA or FPRL1 or a fragment thereof) from an amino acid sequence. Insight II is a three-dimensional graphics program that can interface with several modules that perform numerous structural analysis and enable real-time rational drug design and combinatorial chemistry. Modules such as Builder, Biopolymer, Consensus, and Converter, for example, allow one to rapidly create a two dimensional or three dimensional model of a polypeptide, carbohydrate, nucleic acid, chemical or combinations of the foregoing from their sequence or structure. The modeling tools associated with Insight II support many different data file formats including Brookhaven and Cambridge databases; AMPAC/MOPAC and QCPE programs; Molecular Design Limited Molfile and SD files, Sybel Mol2 files, VRML, and Pict files.

Additionally, the techniques described above can be supplemented with techniques in molecular biology to design models of the protein of interest. For example, a peptide of interest (e.g., FPRL1, SAA and/or fragments thereof) can be analyzed by an alanine scan (Wells, Methods in Enzymol. 202:390-411 (1991)) or other types of site-directed mutagenesis analysis. In alanine scan, each amino acid residue of the polypeptide of interest is sequentially replaced by alanine in a step-wise fashion (i.e., only one alanine point mutation is incorporated per molecule starting at position #1 and proceeding through the entire molecule), and the effect of the mutation on the peptide's activity in a SAA/FPRL1 characterization assay is determined. Each of the amino acid residues of the peptide is analyzed in this manner and the regions important for assembly of the SAA/FPRL1 complex are identified. These functionally important regions can be recorded on a computer readable medium, stored in a database in a computer system, and a search program can be employed to generate a protein model of the functionally important regions.

Once a model of the polypeptide of interest (e.g., SAA, FPRL1, or a fragment thereof) is created, it can be compared to other models so as to identify new agents (“candidate agents”) that can effect assembly of the SAA/FPRL1 complex. By starting with the amino acid sequence or protein model, for example, ligands having two-dimensional and/or three-dimensional homology can be rapidly identified. In one approach, a percent sequence identity can be determined by standard methods that are commonly used to compare the similarity and position of the amino acid of two polypeptides. Using a computer program such as BLAST or FASTA, two polypeptides can be aligned for optimal matching of their respective amino acids (either along the full length of one or both sequences, or along a predetermined portion of one or both sequences). Such programs provide “default” opening penalty and a “default” gap penalty, and a scoring matrix such as PAM 250 (a standard scoring matrix; see Dayhoff et al., in: Atlas of Protein Sequence and Structure, Vol. 5, Supp. 3 (1978)) can be used in conjunction with the computer program. The percent identity can then be calculated as:

$$\frac{\text{total number of identical matches}}{\begin{matrix}[ \text{length of the longer sequence within the matched span} + \\ \text{number of gaps introduced into the longer sequence} \\ \text{in order to aligh the two sequences} ]\end{matrix}}$$

Accordingly, the protein sequence corresponding to SAA or FPRL1 or fragments thereof can be compared to known sequences on a protein basis. Protein sequences corresponding to SAA and FPRL1 are compared, for example, to known amino acid sequences found in Swissprot release 35, PIR release 53 and Genpept release 108 public databases using BLASTP with the parameter W=8 and allowing a maximum of 10 matches. In addition, the protein sequences encoding SAA are compared to publicly known amino acid sequences of Swissprot using BLASTX with the parameter E=0.001. The candidate ligands desirably have at least 50% homology and preferably have 60% or 70% or 80% or 90% or greater homology to SAA or FPRL1. The candidate ligands can have the following degrees of homology to SAA or FPRL1, for example: 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%. The candidate ligands having greater than or equal to 50% homology are identified and are subsequently examined using the SAA/FPRL1 characterization assays described herein. Candidate ligands that can effect assembly of the SAA/FPRL1 complex and/or modulate SAA/FPRL1-mediated signal transduction are, thus, identified.

In another embodiment, computer modeling and the sequence-to-structure-to-function paradigm is exploited to identify more FPRL1 modulating agents that effect assembly of the SAAIFPRL1 complex and/or modulate SAA/FPRL1-mediated signal transduction. By this approach, first the structure of a modulator (e.g., SAA, FPRL1, or a fragment thereof) having a known response in a SAA/FPRL1 characterization assay is determined from its sequence using a threading algorithm, which aligns the sequence to the best matching structure in a structural database. Next, the protein's active site (i.e., the site important for a desired response in the characterization assay) is identified and a “fuzzy functional form” (FFF)—a three-dimensional descriptor of the active site of a protein—is created. (See e.g., Fetrow et al., J. Mol. Biol. 282:703-711 (1998) and Fetrow and Skolnick, J. Mol Biol. 281: 949-968 (1998).

The FFFs are built by iteratively superimposing the protein geometries from a series of functionally related proteins with known structures. The FFFs are not overly specific, however, and the degree to which the descriptors can be relaxed is explored. In essence, conserved and functionally important residues for a desired response are identified and a set of geometric and conformational constraints for a specific function are defined in the form of a computer algorithm. The program then searches experimentally determined protein structures from a protein structural database for sets of residues that satisfy the specified constraints. In this manner, homologous three-dimensional structures can be compared and degrees (e.g., percentages of three-dimensional homology) can be ascertained. The ability to search three-dimensional structure databases for structural similarity to a protein of interest can also be accomplished by employing the Insight II using modules such as Biopolymer, Binding Site Analysis, and Profiles-3D.

By using this computational protocol, genome sequence data bases such as maintained by various organizations including: tigr.org/tdb, genetics.wisc.edu, stanford.edu/˜ball, hiv-web.lanl.gov, ncbi.nlm.nih.gov, ebi.ac.uk, patteur.fr/other/biology, and genome.wi.mit.edu, can be rapidly screened for specific protein active sites and for identification of the residues at those active sites that resemble a desired molecule. Several other groups have developed databases of short sequence patterns or motifs designed to identify a given fimction or activity of a protein. Many of these databases, notably Prosite (expasy.hcuge.ch/sprot/prosite), Blocks (blocks.fhcrc.org), and Prints (biochem.ucl.ac.uk/bsm/dbfrowser/PRINTS/PRINTS), the Molecular Modelling Database (MMDB), and the Protein Data Bank can use short stretches of sequence information to identify sequence patterns that are specific for a given function; thus they avoid the problems arising from the necessity of matching entire sequences. In this manner, new modulating agents are rationally selected for further identification by SAA/FPRL1 characterization assays, as described above. Rounds or cycles of fimctional assays on the molecules and derivatives thereof and further FFF refinement and database searching allows an investigator to more narrowly define classes of modulating agents that produce a desired effect on assembly of the SAA/FPRL1 complex and/or SAA/FPRL1-mediated signal transduction.

In addition to identifying naturally occuring ligands for SAA and/or FPRL1 or fragments thereof, a three-dimensional structure of these peptides can facilitate the design of derivative molecules that produce a more desirable cellular response. Polypeptide derivatives of the invention can be fragments of SAA or FPRL1 or homologs or mutants of SAA or FPRL1, or polypeptide fusions, modified polypeptides, peptidomimetics, or chemicals. For example, a derivative polypeptide can include a polypeptide that has been engineered to have one or more cystine residues incorporated into the protein so as to promote the formation of a more stable derivative through disulfide bond formation. (See e.g., U.S. Pat. No. 4,908,773). In the past, investigators have employed computers and computer graphics programs to aid in assessing the appropriateness of potential cystine linkage sites. (Perry, L. J., & Wetzel, R., Science, 226:555-557 (1984); Pabo, C. O., et al., Biochemistry, 25:5987-5991 (1986); Bott, R., et al., European Patent Application Ser. No. 130,756; Perry, L. J., & Wetzel, R., Biochemistry, 25:733-739 (1986); Wetzel, R. B., European Patent Application Ser. No. 155,832). The introduction of a cystine residue in a polypeptide can be accomplished using conventional molecular biology.

Additional polypeptide derivatives include peptidomimetics that resemble a polypeptide of interest. The naturally occurring amino acids employed in the biological production of peptides all have the L-configuration. Synthetic peptides can be prepared employing conventional synthetic methods, utilizing L-amino acids, D-amino acids, or various combinations of amino acids of the two different configurations. Synthetic compounds that mimic the conformation and desirable features of a particular peptide, e.g., an oligopeptide, once such peptide has been found, but that avoids the undesirable features, e.g., flexibility (loss of conformation) and bond breakdown are known as a “peptidomimetics”. (See, e.g., Spatola, A. F. Chemistry and Biochemistry of Amino Acids. Peptides, and Proteins (Weistein, B, Ed.), Vol. 7, pp. 267-357, Marcel Dekker, New York (1983), which describes the use of the methylenethio bioisostere [CH₂ S] as an amide replacement in enkephalin analogues; and Szelke et al., In peptides: Structure and Function, Proceedings of the Eighth American Peptide Symposium, (Hruby and Rich, Eds.); pp. 579-582, Pierce Chemical Co., Rockford, Ill. (1983), which describes renin inhibitors having both the methyleneamino [CH₂ NH] and hydroxyethylene [CHOHCH₂] bioisosteres at the Leu-Val amide bond in the 6-13 octapeptide derived from angiotensinogen).

In general, the designing and synthesizing of a peptidomimetic involves starting with the sequence of the peptide and the conformation data (e.g., geometry data, such as bond lengths and angles) of a desired peptide (e.g., the most probable simulated peptide), and using such data to determine the geometries that should be designed into the peptidomimetic. Numerous methods and techniques are known in the art for performing this step, any of which could be used. (See, e.g., Farmer, P. S., Drug Design, (Ariens, E. J. ed.), Vol. 10, pp. 119-143 (Academic Press, New York, London, Toronto, Sydney and San Francisco) (1980); Farmer, et al., in TIPS, 9/82, pp. 362-365; Verber et al., in TINS, 9/85, pp. 392-396; Kaltenbronn et al., in J. Med. Chem. 33: 838-845 (1990); and Spatola, A. F., in Chemistry and Biochemistry of Amino Acids. Peptides, and Proteins, Vol. 7, pp. 267-357, Chapter 5, “Peptide Backbone Modifications: A Structure-Activity Analysis of Peptides Containing Amide Bond Surrogates. Conformational Constraints, and Relations” (B. Weisten, ed.; Marcell Dekker: New York, pub.) (1983); Kemp, D. S., “Peptidomimetics and the Template Approach to Nucleation of .beta.-sheets and .alpha.-helices in Peptides,” Tibech, Vol. 8, pp. 249-255 (1990). Additional teachings can be found in U.S. Pat. Nos. 5,288,707; 5,552,534; 5,811,515; 5,817,626; 5,817,879; 5,821,231; and 5,874,529.

Once protein models of the functionally important regions of SAA and/or FPRL1 have been generated, a database comprising one or more libraries having peptides, chemicals, peptidomimetics and other agents can also be accessed by a search program and selected agents can be compared to the protein models to identify molecules that effect the assembly of the SAA/FPRL1 complex and/or modulate SAA/FPRL1-mediated signal transduction. Desirably, agents identified by the approach above are then tested in the SAA/FPRL1 characterization assays and are used to construct multimeric agents and/or are incorporated into pharmaceuticals. One program that allows for such analysis is Insight II having the Ludi module. Further, the Ludi/ACD module allows a user access to over 65,000 commecially available drug candidates (MDL's Available Chemicals Directory) and provides the ability to screen these compounds for interactions with the protein of interest.

By a similar approach, a modulating agent that interacts with FPRL1 or SAA can be manufactured and identified as follows. First, a molecular model of one or more modulating agents or portions of these molecules that interact with FPRL1 or SAA are created using one of the techniques discussed above or as known in the art. Next, chemical libraries and databases are searched for molecules similar in structure to the known FPRL1 modulating agents. That is, a search can be made of a three dimensional data base for non-peptide (organic) structures (e.g., non-peptide analogs, and/or dipeptide analogs) having three dimensional similarity to the known structure of the target compound. See, e.g., the Cambridge Crystal Structure Data Base, Crystallographic Data Center, Lensfield Road, Cambridge, CB2 1EW, England; and Allen, F. H., et al., Acta Crystallogr., B35: 2331-2339 (1979).

It is noted that search algorithms for three dimensional data base comparisons are available in the literature. See, e.g., Cooper, et al., J. Comput.-Aided Mol. Design, 3: 253-259 (1989) and references cited therein; Brent, et al., J. Comput.-Aided Mol. Design, 2: 311-310 (1988) and references cited therein. Commercial software for such searches is also available from vendors such as Day Light Information Systems, Inc., Irvine, Calif. 92714, and Molecular Design Limited, 2132 Faralton Drive, San Leandro, Calif. 94577. The searching is done in a systematic fashion by simulating or synthesizing analogs having a substitute moiety at every residue level. Preferably, care is taken that replacement of portions of the backbone does not disturb the tertiary structure and that the side chain substitutions are compatible to retain the receptor substrate interactions.

Once candidate agents have been identified, desirably, they are analyzed in a SAA/FPRL1 characterization assay. The assays, described herein and assays that evaluate the formation of a SAA/FPRL1 complex in the presence of modulators, as will be apparent to one of skill in the art given the disclosure herein (referred to collectively as “FPRL1 characterization assays”), can be performed on the candidate agents and groups of modulating agents (wherein the grouping is based on the potency of modulation of the formation of a SAA/FPRL1 complex and/or SAA/FPRL1-mediated signal transduction) are identified and recorded on a computer readable media. Further cycles of modeling and SAA/FPRL1 characterization assays can be employed to more narrowly define the parameters needed in an optimal FPRL1 modulating agent.

Each FPRL1 modulating agent and its response in a FPRL1 characterization assay can be recorded on a computer readable media and a database or library of FPRL1 modulating agents and respective responses in the SAA/FPRL1characterization assay can be generated. These databases or libraries can be used by researchers to identify important differences between active and inactive molecules so that compound libraries are enriched for modulating agents that have favorable characteristics. Further, enrichment can be achieved by using approaches in dynamic combinatorial chemistry. (See e.g., Angnew, Chem. Int. Ed., 37:2828 (1998)). For example, a target biomolecule, such as FPRL1 or SAA, is joined to a support and is bound by the modulating agents from the libraries generated above. The FPRL1 resin bound with one or more candidate modulating agents is removed from the binding reaction, the modulating agents are eluted from the support, and are identified. Cycles of immobilized target binding assays are conducted, classes of modulating agents that exhibit desired binding characteristics are identified, and this data is recorded on a computer readable media and is used to select more modulating agents that produce a desired modulation of the formation of a SAA/FPRL1 complex and or SAA/FPRL1-mediated signal transduction.

Additionally, agents identified by the approaches described above can be synthesized on solid support beads by split-and-pool synthesis, a multistage process for producing very large numbers of compounds, to facilitate testing. The support-bound agents can be screened in SAA/FPRL1 characterization assays or “free mixtures” are created by cleaving the agent from the support and these free mixtures are screened in the FPRL1 characterization assays. Compounds that produce desirable responses are identified, recorded on a computer readable media, and the process can be repeated to select optimal FPRL1 modulating agents.

Many computer programs and databases can be used with embodiments of the invention to identify agents that modulate assembly of the SAA/FPRL1 complex and/or SAA/FPRL1-mediated signal transduction. The following list is intended not to limit the invention but to provide guidance to programs and databases that are useful with the approaches discussed above. The programs and databases that can be used include, but are not limited to: MacPattern (EMBL), DiscoveryBase (Molecular Applications Group), GeneMine (Molecular Applications Group), Look (Molecular Applications Group), MacLook (Molecular Applications Group), BLAST and BLAST2 (NCBI), BLASTN and BLASTX (Altschul et al, J. Mol. Biol. 215: 403 (1990), herein incorporated by reference), FASTA (Pearson and Lipman, Proc. Natl. Acad. Sci. USA, 85: 2444 (1988), herein incorporated by reference), Catalyst (Molecular Simulations Inc.), Catalyst/SHAPE (Molecular Simulations Inc.), Cerius².DBAccess (Molecular Simulations Inc.), HypoGen (Molecular Simulations Inc.), Insight II, (Molecular Simulations Inc.), Discover (Molecular Simulations Inc.), CHARMm (Molecular Simulations Inc.), Felix (Molecular Simulations Inc.), DelPhi, (Molecular Simulations Inc.), QuanteMM, (Molecular Simulations Inc.), Homology (Molecular Simulations Inc.), Modeler (Molecular Simulations Inc.), Modeller 4 (Sali and Blundell J. Mol. Biol. 234:217-241 (1997)), ISIS (Molecular Simulations Inc.), Quanta/Protein Design (Molecular Simulations Inc.), WebLab (Molecular Simulations Inc.), WebLab Diversity Explorer (Molecular Simulations Inc.), Gene Explorer (Molecular Simulations Inc.), SeqFold (Molecular Simulations Inc.), the EMBL/Swissprotein database, the MDL Available Chemicals Directory database, the MDL Drug Data Report data base, the Comprehensive Medicinal Chemistry database, Derwents's World Drug Index database, and the BioByteMasterFile database. Many other programs and data bases would be apparent to one of skill in the art given the present disclosure.

In the discussion below, we describe several embodiments of the invention that have therapeutic and/ or prophylactic application.

Therapeutic and Prophylactic Applications

In the therapeutic and prophylactic embodiments, the peptide agents identified as modulating the formation of the SAA/FPRL1 complex and/or SAA/FPRL1-mediated signal transduction are incorporated into a pharmaceutical product and are administered to a subject in need. The monomeric and multimeric peptide agents of the invention are suitable for treatment of subjects either as a preventive measure to avoid a particular cellular response or as a therapeutic to treat subjects in need of a particular cellular response.

One contemplated method of making a pharmaceutical involves the selection of a peptide agent that interacts with FPRL1, preferably SAA, a fragment of SAA, or a peptidomimetic that resembles SAA or a fragment thereof, and incorporating the peptide agent into a pharmaceutical by conventional techniques. Additionally, a peptide agent such as FPRL1, a fragment of FPRL1, or a peptidomimetic that resembles FPRL1 or a fragment thereof can be incorporated into a pharmaceutical. The pharmaceuticals of the present invention can be formulated with an adjuvant or can be free and desirable embodiments provide the peptide agent in a support-bound form. Optionally, the peptide agent can be provided in an aggregated form as created, for example, by heating.

In another method of making a pharmaceutical, we envision incorporating a peptide agent selected for its ability to block, inhibit, or prevent a cellular response in the characterization assays described above. Accordingly, a peptide agent that interacts with FPRL1 or SAA and blocks, inhibits, or suspends a cellular response by effecting the assembly of the SAA/FPRL1 complex is identified and is incorporated into a pharmaceutical by conventional techniques. A novel class of peptide agents that bind FPRL1 or SAA with high avidity but fail to induce a cellular response can be designed using approaches described above. These pharmaceuticals are formulated in adjuvant or free and are provided in the form of a support-bound agent, as well. As above, an aggregated form of this embodiment can be created by heating the proteins and can administered to subjects in need.

The compounds of this invention can be employed in admixture with conventional excipients, i.e., pharmaceutically acceptable organic or inorganic carrier substances suitable for parenteral, enteral (e.g., oral) or topical application which do not deleteriously react with the active compounds. Suitable pharmaceutically acceptable carriers include but are not limited to water, salt solutions, alcohols, gum arabic, vegetable oils, benzyl alcohols, polyetylene glycols, gelatine, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds. They can also be combined where desired with other active agents, e.g., vitamins. The ligands can also be administered in the form of a support-bound agent or in a pro-drug form which interacts with a support so as to create a support-bound agent in the body of the subject. Further, the manufacture of pharmaceuticals or therapeutic agents that deliver the peptide agent and/or a nucleic acid sequence encoding the peptide agent by several routes is another aspect of the invention. For example, and not by way of limitation, the use of DNA, RNA, and viral vectors having sequence encoding the peptide agent is contemplated. Nucleic acids encoding a desired peptide agent can be administered alone or in combination with peptide agents.

In the following disclosure, doses and methods of administration are provided.

Dosage and Methods of Administration

The effective dose and method of administration of a particular formulation of a peptide agent can vary based on the individual subject and the stage of the disease, as well as other factors known to those of skill in the art. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions which exhibit large therapeutic indices are preferred. The data obtained from cell culture assays and animal studies is used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Additional factors which can be taken into account include the severity of the disease state, age, weight and gender of the patient; diet, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

Normal dosage amounts can vary from 0.1 to 100,000 micrograms, up to a total dose of about 10 grams, depending upon the route of administration. Guidance as to particular dosages and methods of delivery is provided in the literature. (See U.S. Pat. Nos. 4,657,760; 5,206,344; or 5,225,212.) More specifically, the dosage of the peptide agents of the present invention is one that provides sufficient peptide agent to attain a desirable effect including an up-regulation or a down regulation of a cellular response. A constant infusion of the peptide agent can also be provided so as to maintain a stable concentration in the tissues as measured by blood levels.

Routes of administration of the peptide agents include, but are not limited to, topical, transdermal, parenteral, gastrointestinal, transbronchial, and transalveolar. Topical administration is accomplished via a topically applied cream, gel, rinse, etc. containing a peptide agent. Transdermal administration is accomplished by application of a cream, rinse, gel, etc. capable of allowing the peptide agent to penetrate the skin and enter the blood stream. Parenteral routes of administration include, but are not limited to, electrical or direct injection such as direct injection into a central venous line, intravenous, intramuscular, intraperitoneal or subcutaneous injection. Gastrointestinal routes of administration include, but are not limited to, ingestion and rectal. Transbronchial and transalveolar routes of administration include, but are not limited to, inhalation, either via the mouth or intranasally.

Compositions of peptide agent-containing compounds suitable for topical application include, but not limited to, physiologically acceptable implants, ointments, creams, rinses, and gels. Any liquid, gel, or solid, pharmaceutically acceptable base in which the peptide agents are at least minimally soluble is suitable for topical use in aspects of the present invention. For topical application, there are also employed as non-sprayable forms, viscous to semi-solid or solid forms comprising a carrier compatible with topical application and having a dynamic viscosity preferably greater than water. Suitable formulations include but are not limited to solutions, suspensions, emulsions, creams, ointments, powders, liniments, salves, aerosols, etc., which are, if desired, sterilized or mixed with auxiliary agents, e.g., preservatives, stabilizers, wetting agents, buffers or salts for influencing osmotic pressure, etc. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., a freon.

Compositions of the peptide agents suitable for transdermal administration include, but are not limited to, pharmaceutically acceptable suspensions, oils, creams, and ointments applied directly to the skin or incorporated into a protective carrier such as a transdermal device (“transdermal patch”). Examples of suitable creams, ointments, etc. can be found, for instance, in the Physician's Desk Reference. Examples of suitable transdermal devices are described, for instance, in U.S. Pat. No. 4,818,540 issued Apr. 4, 1989 to Chinen, et al.

Compositions of the peptide agents suitable for parenteral administration include, but are not limited to, pharmaceutically acceptable sterile isotonic solutions. Such solutions include, but are not limited to, saline and phosphate buffered saline for injection into a central venous line, intravenous, intramuscular, intraperitoneal, or subcutaneous injection of the peptides. Additional embodiments for parenteral application include injectable, sterile, oily solutions, suspensions, emulsions, or implants, including suppositories. Ampoules are convenient unit dosages.

Compositions of the peptide agents suitable for transbronchial and transalveolar administration include, but not limited to, various types of aerosols for inhalation. Devices suitable for transbronchial and transalveolar administration of the peptide agents are also embodiments. Such devices include, but are not limited to, atomizers and vaporizers. Many forms of currently available atomizers and vaporizers can be readily adapted to deliver peptide agents.

Compositions of the peptide agents suitable for gastrointestinal administration include, but not limited to, pharmaceutically acceptable powders, tablets, pills, dragees, capsules, drops, or liquids for ingestion and suppositories for rectal administration. A syrup, elixir, or the like can be used wherein a sweetened vehicle is employed.

Sustained, pro-drugs, or directed release compositions can be formulated, e.g., liposomes or those wherein the active compound is protected with differentially degradable coatings, e.g., by microencapsulation, multiple coatings, etc. It is also possible to freeze-dry the ligands and use the lyophilizates obtained, for example, for the preparation of products for injection.

Aspects of the invention also include a coating for medical equipment. Alternatively, the peptides can be impregnated into a polymeric medical device such as catheters, stents and prosthetics. Coatings suitable for use in medical devices can be provided by a powder containing the peptides or by polymeric coating into which the peptides are suspended. Suitable polymeric materials for coatings or devices are those which are physiologically acceptable and through which a therapeutically effective amount of the peptide agent can diffuse. Suitable polymers include, but are not limited to, polyurethane, polymethacrylate, polyamide, polyester, polyethylene, polypropylene, polystyrene, polytetrafluoroethylene, polyvinyl-chloride, cellulose acetate, silicone elastomers, collagen, silk, etc. Such coatings are described, for instance, in U.S. Pat. No. 4,612,337, issued Sep. 16, 1986 to Fox et al. which is incorporated herein by reference.

It will be appreciated that the actual preferred amounts of active compound in a specific case will vary according to the specific compound being utilized, the particular compositions formulated, the mode of application, and the particular situs and organism being treated. The materials and methods and the experiments presented above are detailed in Su et al., J. Exp. Med., 189 (2):395-402 (1999). The example below discloses the materials and methods used to perform many of the experiments described above.

### EXAMPLE 1

Reagents and Cells.

Recombinant human (rh) SAA was purchased from Pepro Tech Inc. (Rocky Hill, N.J.) with the sequence as follows:

MRSFFSFLGEAFDGARDMWRAYSDMREANYIGSDKYFHARGNYD AAKRGPGGVWAAEAISNARENIQRFFGRGAEDSLADQAANEWGRSGKDPNHFRPA GLPEKY. (SEQ. ID. No. 1). This rhSAA corresponds to SAA-1α, one of the major SAA isoforms in the serum, except for the addition of a methionine at the NH2 terminus as well as the substitution of aspartic acid for asparagine at position 60, which appears in the SAA2 isoform (reviewed in ref. Steinkasserer et al., Biochem. J., 268:187-193 (1990)). rhSAA at concentrations used in the study was negative for endotoxin as assessed by Limulus amebocyte lysate assays (sensitivity: 0.06 IU/ml. Bio Whittaker, Walkersville, Md.). High density lipoprotein (HDL) was purchased from Sigma (St. Louis, Mo.). Human peripheral blood enriched in mononuclear cells or neutrophils was obtained from normal donors by leukapheresis (courtesy of Transfusion Medicine Department, Clinical Center, National Institutes of Health, Bethesda, Md.). The blood was centrifuged through Ficoll-Hypaque (Sigma) and mononuclear cells (PBMC) collected at the interphase were washed with PBS and centrifuged through a 46% isoosmotic Percoll (Pharmacia, Uppsala, Sweden) gradient followed by elutriation to yield monocytes (purity: >90%). Neutrophils were purified by 3% dextran/PBS sedimentation as described elsewhere (Badolato et al., J. Exp. Med., 180:203-209 (1994)) and were more than 98% pure. The cells were resuspended in RPMI 1640 medium containing 10% FCS (Hyclone, Logan, Utah) for future use. The molecular cloning of the receptors for fMLP was described previously (Murphy et al., J. Biol. Chem., 267:7637-7643 (1992); Gao, J. L. and P. M. Murphy, J. Biol. Chem., 268:25395-25401 (1993); Murphy, P. M. and D. McDermott, J. Biol. Chem., 266:12560-12567 (1991); and Ali et al., J. Biol. Chem., 273:11012-11016 (1998)). The cDNAs encoding classical formyl peptide receptor FPR and its variant FPRL1 were stably transfected into human embryonic kidney epithelial cell line 293 that were cultured in DMEM in the presence of 800: g/ml geneticin (G418, GibcoBRL, Grand Island, N.Y.) to maintain selection. A rat basophil leukemia cell line stably transfected with FPR (ETFR cells ) was also used in the study (a kind gift from Drs. H. Ali and R. Snyderman, Duke University Medical Center, NC).

Chemotaxis.

The migration of human 293 cells expressing FPR (FPR/293) or FPRL1(FPRL1/293) as well as ETFR cells was assessed by a 48-well microchemotaxis chamber technique (Falk et al., J. Immunol. Methods, 33:239-247 (1980); and Gong et al., J. Biol. Chem. 272:11682-11685 (1997)). A 25:1 aliquot of rhSAA or other reagents diluted in chemotaxis medium (RPMI1640, 1% BSA, 25 mM HEPES) was placed in the wells of the lower compartment, and 50:1 cell suspension (1×10^{≢}cell/ml in chemotaxis medium) were placed in the wells of the upper compartment of the chamber (Neuroprobe, Cabin John, Md.). The two compartments were separated by a polycarbonate filter (10: m pore size, Neuroprobe) coated with 50: g/ml collagen type I (GIBCO, Gaithersburg, MD) for 1 h at 37° C. The chamber was incubated at 37° C. for 5 h in humidified air with 5% CO2. At the end of the incubation, the filter was removed, fixed and stained with Diff-Quik (Harlew, Gibbstown, N.J.). The number of migrated cells in three high-powered fields (400×) were counted by light microscopy after coding the samples. Results are expressed as the mean (±SD) value of the migration in triplicate samples and are representative of at least 5 experiments performed. For better illustration, chemotaxis indexes (CI) reflecting the fold increase of cell migration in response to stimulant over medium are used. Statistical significance of the difference between numbers of cells migrating in response to stimuli versus baseline (migration toward control medium ) was calculated with Student's T test and the CI≧2 are statistically significant.

Calcium Mobilization.

Calcium mobilization was assayed by incubating 10⁷/ml of monocytes, neutrophils or receptor cDNA transfectants in loading buffer containing 138 mM NaCl, 6 mM KCl, 1 mM CaCl₂, 10 mM HEPES (pH 7.4), 5 mM Glucose, 0.1% BSA with 5: M Fura-2 (Sigma) at 37° C. for 30 min. The dye-loaded cells were washed and resuspended in fresh loading buffer. The cells were then transfered into quartz cuvettes (10⁶ cells in 2 ml) which were placed in a luminescence spectrometer LS50 B (Perkin-Elmer Limited, Beaconsfield, England). Stimulants at different concentrations were added in a volume of 20: l to the cuvettes at indicated time points. The ratio of fluorescence at 340 and 380 nm wavelength was calculated using the FL WinLab program. The assays were performed at least 5 times and results from representive experiments are shown.

Ligand Binding Assays.

rhSAA (20: g) was radio-iodinated on tyrosine and lysine residues with the chloramine T method and the specific activity of the labeled SAA was 5.8 mCi/mg (courtesy of J. Dobbs, SAIC Frederick, NCI-FCRDC, Frederick, Md.). A constant concentration of 16 nM ¹²⁵I-SAA was incubated for 20 min at 37° C. with human monocytes or 293 cells transfected with chemoattractant receptor cDNAs (1.5-2×10⁶/sample, in 200: l RPMI1640, 1% BSA, 0.05% NaN3) in the presence of increasing concentrations of unlabeled SAA. After incubation, the cells were washed once with ice-cold PBS then were layered onto a 10% sucrose/PBS cushion in Eppendorf tubes. The cells were centrifuged at 10,000 g for 1 min and the tips of the tubes containing cell pellets were cut and measured for radioactivity in a γ-counter. The binding data were analyzed and plotted with a computer-aided program LIGAND (P. Munson, Division of Computer Research and Technology, NIH, Bethesda, Md.). The level of specific binding was determined by subtraction of non-specific binding (cpm on cells in the presence of 1: M unlabeled SAA) from the total binding (cpm on cells in the absence of unlabeled SAA). Experiments were performed at least 5 times which yielded similar results.

Although the invention has been described with reference to embodiments and examples, it should be understood that various modifications can be made without departing from the spirit of the invention. Accordingly, the invention is limited only by the following claims.

1-28. (canceled)

29. A method of identifying a compound that modulates binding of SAA (serum amyloid A) to a FPRL1 (formyl peptide receptor-like 1) receptor comprising:
(a) providing a test compound that is a peptide, peptidomimetic, or chemical;
(b) contacting a cell that expresses a FPRL1 receptor with the test compound in the presence of SAA; and
(c) measuring binding of SAA to said FRPL1 receptor, so that a compound that modulates binding of SAA to said FRPL1 receptor is identified.

30. The method of claim 29 wherein said test compound is a peptide.

31. The method of claim 29 wherein said test compound is a peptidomimetic.

32. The method of claim 29 wherein said test compound is a chemical.

33. The method of claim 30 wherein said peptide is a carboxy truncation, amino truncation, or internal truncation of SAA having a sequence selected from the group consisting of SEQ ID NOs: 2-301.

34. The method of claim 30 wherein said peptide is SAA.

35. The method of claim 30 wherein said cell is a genetically engineered host cell that contains a sequence encoding a FRPL1 receptor so that a FPRL1 receptor is stably expressed by the host cell.

36. The method of claim 30 wherein the SAA is labeled.

37. The method of claim 30 wherein a compound that inhibits binding of SAA to said FRPL1 receptor is identified.

38. The method of claim 30 further comprising incorporating the compound that is identified into a composition.
